(19) United States
(12) Patent Application Publication
(10) Pub. No.: US 2026/0250629 A1
(43) Pub. Date: Aug. 27, 2026

(54) STEM CELLS BASED THREE-DIMENSIONAL EMBRYO MODEL

(71) Applicant: CAMBRIDGE ENTERPRISE LIMITED, Cambridge (GB)

(72) Inventors: Azim SURANI, Cambridge (GB); Jitesh NEUPANE, Cambridge (GB)

(21) Appl. No.: 19/489,726

(22) PCT Filed: Jun. 6, 2024
(86) PCT No.: PCT/GB2024/051458

§ 371 Date: Dec. 3, 2025

(30) Foreign Application Priority Data
Jun. 6, 2023 (GB) 2308408.0

(51) Int. Cl. C12N 5/073

(52) U.S. Cl. CPC C12N 5/0604; C12N 2500/02; C12N 2501/15; C12N 2501/16; C12N 2501/727; C12N 2506/02

(57) ABSTRACT

The present invention relates to an in vitro human embryo organoid at a post-gastrulation stage of development. Methods of forming the organoid and derivatives therefrom are described as well as their uses. The in vitro human embryo organoid at a post-gastrulation stage of development demonstrates early human development that recapitulates the formation of three germ layers, and hPGCLCs without exogenous BMP signalling.

### FIELD OF INVENTION

The present invention relates to an in vitro human embryo organoid and methods of producing the same. More particularly, the invention relates to an in vitro human embryo organoid at a post-gastrulation stage of development and methods of producing the same.

### BACKGROUND

The classical Carnegie collection of human embryology from the 19^{th} and 20^{th} centuries^{1,2} has provided an essential foundation for the landmarks of human development. However, these do not provide details or mechanistic insights into critical events of very early development and disorders. More recently, in vitro models described as blastoids³⁻⁶, embryoids^{7,8} and gastruloids⁹⁻¹³, primarily with murine but also human embryonic stem cells (hESCs), apparently mimic pre- and post-gastrulation embryonic development. Bioengineering approaches have also resulted in micropatterned embryos^{14,15} and the use of microfluidics for developing posterior epiblast and amniogenesis^{16,17}. All these models have strengths and weaknesses, biases toward particular lineages, and barriers that prevent cultures for prolonged periods, but overall led to significant advances in the field.

The need for models that constitute early human development is particularly acute because it is difficult to access early human embryos. The human body plan is established following blastocyst implantation on embryonic days 6-7 (E6-7), starting as a bilaminar epiblast-hypoblast disc, followed by a trilaminar disc comprising the three germ layers: ectoderm, mesoderm and endoderm. During gastrulation, epiblast cells invaginate and undergo epithelial-to-mesenchymal transition (EMT), giving rise to primitive streak (PS) and the formation of mesoderm and endoderm. Primordial germ cells (PGCs), the precursors of sperm and oocytes, also appear during gastrulation^{18,19}. The appearance of neuronal precursors and the onset of organogenesis, including the heart, are among the first to develop after gastrulation at about week four, followed by fetal haematopoiesis.

Here, inventors present a highly tractable and reproducible approach for generating synthetic human embryos from hESCs encompassing gastrulation and post-gastrulation development by combining static and kinetic cultures for twenty-two days, which are referred to as human embryo organoids (henceforth termed as hEOs). The model depicts early human development that recapitulates the formation of three germ layers, and hPGCLCs without exogenous BMP signalling. Notably, hEOs develop significantly beyond gastrulation, with the onset of organogenesis, including haematopoiesis and a beating heart.

### SUMMARY OF THE INVENTION

According to a first aspect of the invention, there is provided, an in vitro human embryo organoid at a post-gastrulation stage of development comprising cells expressing markers characteristic of endodermal cells, cells expressing markers characteristic of mesodermal cells, cells expressing markers characteristic of ectodermal cells, and primordial germ cell-like cells (hPGCLCs).

In one embodiment, the in vitro human embryo organoid further comprises one or more of neuromesodermal progenitors, a heart tube, a beating heart, hindgut, neural tube, neural crest cells, neuronal progenitors, hematopoietic cells, endothelial cells, hepatocytes, and cardiomyocytes.

In one embodiment, the in vitro human embryo organoid is non-integrated.

In one embodiment, in vitro human embryo organoid is cultured for any one of at least 3, 4, 5, 6, 7, 8, 9, 10, 11, 12, 13, or 14 days.

In one embodiment, in vitro human embryo organoid is polarised along the rostro-caudal axis into an anterior region and a posterior region.

In one embodiment, the anterior region and posterior regions comprise differential expression of one or more one or more markers indicative of primordial germ cell-like cells.

In one embodiment, GATA6 gene is expressed in the anterior region and TBXT gene or CDX2 gene is expressed at the posterior region.

In one embodiment, the anterior region comprises upregulation or downregulation of one or more genes selected from a list comprising GATA6, MYH10, TNNI1, TNNI2, and HAND1 and/or the posterior region comprises upregulation or downregulation of one of more genes selected from a list comprising CDX2 and CDX1.

In one embodiment, the posterior region comprises markers for neuromesodermal progenitors, wherein the markers for neuromesodermal progenitors are one or more genes selected from a list comprising TBXT, SOX2, NKX1.2, HOXB8, and HOXC9.

In one embodiment, human embryo organoid comprises neural precursors, wherein the neural precursors are one or more genes selected from a list comprising SOX2, ZIC5, POU3F2, neural progenitors, wherein the neural progenitors are one or more genes selected from a list comprising ISI1, ZEB2, FOXC1, neural crest markers, wherein the neural crest are one or more genes selected from a list comprising FOXD3, S100B, HOXB9, SOX2, SOX10, CRABP1, TFAP2A, TFAP2B, neuron markers, wherein the neuron markers are one or more genes selected from a list comprising STMN2, ASCL1, ELAVL3, NEUROG1, and NEUROD4, neural tube markers, wherein the neural tube markers are one or more genes selected from a list comprising SOX3, PAX6, PAX7, and HES5, spinal cord markers, wherein the spinal cord markers are one or more genes selected from a list comprising OLIG3, SOX1, ZIC1, PAX7, and FGF8, and neural ectoderm markers, wherein the neural ectoderm markers are one or more genes selected from a list comprising SOX1 and PAX6.

In one embodiment, the in vitro human embryo organoid comprises Hematopoietic Stem and Progenitor Cells (HSPC), wherein the HSPCs comprise one or more genes selected from a list comprising RUNX1, SOX7 and CD34.

In one embodiment, the in vitro human embryo organoid comprises one or more selected from a list comprising hematopoietic cells CD45⁺RUNX1⁺, RUNX1⁺CD45⁻, and CD45⁺RUNX1⁻CD31⁺CD45⁻, CD31⁺CD45⁺, CD31⁻CD45⁺, CD31⁺CD34⁺CD45⁺, CD34⁺CD45⁻ and CD34⁻CD45⁺.

In one embodiment, the hematopoietic cells further comprise one or more genes selected from a list comprising CD45, MYB, ITGA2B, SPN, GFI1B, SPINK2 and CD44.

In one embodiment, the hematopoietic cells further comprise markers for megakaryocytes, wherein the markers for megakaryocytes comprise one or more genes selected from a list comprising GP1BA, GP1BB and NFE2.

In one embodiment, the hematopoietic cells further comprise markers for macrophages, wherein the markers for macrophages comprise one or more genes selected from a list comprising CD68, CD86, MRC1 and TREM2.

In one embodiment, the hematopoietic cells further comprise markers for eosinophil, basophil and mast cell progenitors, wherein the eosinophil, basophil and mast cell progenitors comprise one or more genes selected from a list comprising GATA2, IL1B, MYC, STAT5A and SOX4.

In one embodiment, the hematopoietic cells further comprise one or more genes selected from a list comprising lymphocytes CD52, CD69, HOPX, SPINK2, IL7R and NKG7.

In one embodiment, the in vitro human embryo organoid expresses one or more markers indicative of primordial germ cell-like cells selected from a group comprising NANOS3, POU5F1, PDPN, TFAP2C, PRDM1, PRDM14, CD38, and NANOG.

In one embodiment, the in vitro human embryo organoid expresses one or more markers indicative of neuronal precursor cells selected from a group comprising S100B, FOXD3, SOX10, ELAVL3, NEUROD4, NEUROG1, SOX2, SOX10, CRABP1, TFAP2A, TFAP2B, STMN2, ASCL1, ELAVL3, NEUROG1, NEUROD4, SOX3, PAX6, PAX7, HES5, OLIG3, SOX1, ZIC1, PAX7, and FGF8.

In one embodiment, the anterior region expresses TNNT2 and MYH2 genes.

In one embodiment, the in vitro human embryo organoid comprises contractile cardiomyocytes, wherein the markers for cardiomyocytes are one or more genes selected from a list comprising NKX2-5 and MYH2, TNNI1, ACTC1, TNNT2, MYH6, MYL7 and IRX3.

In one embodiment, the in vitro human embryo organoid comprises amnion cells, wherein the markers for amnion cells are one or more genes selected from a list comprising GABRP, VTCN1, WNT6 and TFAP2A,

In one embodiment, the in vitro human embryo organoid comprises mesenchyme cells, wherein the markers for mesenchyme cells are one or more genes selected from a list comprising PCOLCE, COL1A2, COL6A3 and HAND1.

In one embodiment, the in vitro human embryo organoid comprises hepatocytes, wherein the markers for hepatocytes are one or more genes selected from a list comprising AFP, ALB and CEBPA.

In one embodiment, the in vitro human embryo organoid comprises blood progenitors, wherein the markers for blood progenitors are one or more genes selected from a list comprising PECAM1/CD31, CD34, CDH5, RUNX1, PTPRC/CD45 and CD44.

In one embodiment, the in vitro human embryo organoid comprises splanchnic mesoderm, wherein the markers for splanchnic mesoderm are one or more genes selected from a list comprising WT1, LHX2 and GATA4,

In one embodiment, the in vitro human embryo organoid comprises paraxial mesoderm, wherein the markers for paraxial mesoderm are one or more genes selected from a list comprising MEOX1, SIX4 and FOXC2.

In one embodiment, the in vitro human embryo organoid comprises erythroid cells, wherein the markers for erythroid cells are one or more genes selected from a list comprising GATA1, KLF1, HBA1, HBA2, HBE1, HBE2, HBG1 and HBG2,

In one embodiment, the in vitro human embryo organoid comprises endothelial cells, wherein the markers for endothelial cells are one or more genes selected from a list comprising CD34, CD31, CDH5, KDR, SOX7, THY1, ITGB1, CXCR4, NRARP, GJA4, IGFBP4, VEGF1, FLT1, TMEM88, TPM1, PRTG, HOXB7, MEIS2, RNF19A, CAV1, REN, BNIP3, STC1 and FGF23.

In one embodiment, the in vitro human embryo organoid comprises hepatocytes (foetal liver progenitors), wherein the markers for hepatocytes are one or more genes selected from a list comprising AFP, APOA1, APOB, ALB and TF.

In one embodiment, the in vitro human embryo organoid comprises musculoskeletal precursors, wherein the musculoskeletal precursors are one or more genes selected from a list comprising NEFM, SIX1 and TNNT1.

In one embodiment, the in vitro human embryo organoid comprises hindgut, wherein the markers for hindgut are one or more genes selected from a list comprising CDX2, SOX2, SOX17, SHH and EPCAM.

In one embodiment, the in vitro human embryo organoid is derived in vitro from one or more human embryonic stem cells (ESCs).

In one embodiment, the in vitro human embryo organoid is derived in vitro from one or more human induced pluripotent stem cells (iPSCs).

In one embodiment, the in vitro human embryo organoid is derived in vitro from one or more stem cell selected from a list comprising naive stem cells, expanded pluripotent stem cells, reset stem cells, formative cells, induced pluripotent stem cells, primed pluripotent stem cells, 8 cell-like embryo-like cells (8CL), 4 cell-like embryo-like cells (4CL).

According to a second aspect of the invention, there is provided a method of producing an in vitro human embryo organoid at a post-gastrulation stage of development comprising the steps of:
a) treating human embryonic stem cells or human induced pluripotent stem cells to disassociate them into single cells,
b) formation of aggregates,
c) advancement of aggregates to human embryo organoid at the pre-gastrulation stage of development; and
d) advancement of aggregates to human embryo organoid at the post-gastrulation stage of development, wherein the aggregates are incubated in the presence of a GSK3β inhibitor, preferably wherein the GSK3β inhibitor is Chiron, and a TGF-β inhibitor, preferably wherein the TGF-β inhibitor is SB43.

In one embodiment, step a) is performed in the presence of a TGF-β, preferably wherein the TGF-β is Activin-A, and a GSK3β inhibitor, preferably wherein the GSK3β inhibitor is Chiron.

In one embodiment, step b) is performed in the presence of a GSK3β inhibitor, preferably wherein the GSK3β inhibitor is Chiron.

In one embodiment, step c) is performed in the presence of a GSK3β inhibitor, preferably wherein the GSK3β inhibitor is Chiron, and a TGF-β inhibitor, preferably wherein the TGF-β inhibitor is SB43.

In one embodiment, step d) further comprises incubating the in vitro human embryo organoid in a rotary culture system.

In one embodiment, one or more of the method steps are performed under conditions of about 1% to about 10% oxygen and 1% to about 6% carbon dioxide, preferably about 5% oxygen and about 5% carbon dioxide, optionally, wherein all of the method steps are performed under conditions of about 1% to about 10% oxygen and 1% to about 6% carbon dioxide, preferably about 5% oxygen and about 5% carbon dioxide.

According to a third aspect of the invention, there is provided a method of advancing an in vitro human embryo organoid at the peri-gastrulation stage of development to a post-gastrulation stage of development comprising incubating the human embryo organoid at the peri-gastrulation stage of development in the presence of a GSK3β inhibitor, preferably wherein the GSK3β inhibitor is Chiron, and a TGF-β inhibitor, preferably wherein the TGF-β inhibitor is SB43.

In one embodiment, the third aspect of the invention further comprising incubating the in vitro human embryo organoid in a rotary culture system.

In one embodiment, one or more of the method steps are performed under conditions of about 1% to about 10% oxygen and 1% to about 6% carbon dioxide, preferably about 5% oxygen and about 5% carbon dioxide, optionally, wherein all of the method steps are performed under conditions of about 1% to about 10% oxygen and 1% to about 6% carbon dioxide, preferably about 5% oxygen and about 5% carbon dioxide.

In one embodiment, the method of producing an in vitro human embryo organoid at a post-gastrulation stage of development according to the second aspect of the invention or the method of advancing an in vitro human embryo organoid at the peri-gastrulation stage of development to a post-gastrulation stage of development according to the third aspect of the invention, the in vitro human embryo organoid comprises:
cells expressing markers characteristic of endodermal cells,
cells expressing markers characteristic of mesodermal cells,
cells expressing markers characteristic of ectodermal cells; and
primordial germ cell-like cells (hPGCLCs).

In one embodiment, the in vitro human embryo organoid further comprises one or more of neuromesodermal progenitors, a heart tube, a beating heart, hindgut, neural tube, neural crest cells, neuronal progenitors, hematopoietic cells, endothelial cells, hepatocytes, and cardiomyocytes indicative of a post-gastrulation state of development.

In one embodiment, in vitro human embryo organoid is cultured for any one of at least 3, 4, 5, 6, 7, 8, 9, 10, 11, 12, 13, or 14 day.

In one embodiment, in vitro human embryo organoid is polarised along the rostro-caudal axis into an anterior region and a posterior region.

In one embodiment, the anterior region and posterior regions comprise differential expression of one or more one or more markers indicative of primordial germ cell-like cells.

In one embodiment, GATA6 gene is expressed in the anterior region and TBXT gene or CDX2 gene is expressed at the posterior region.

In one embodiment, the anterior region comprises upregulation or downregulation of one of more genes selected from a list comprising GATA6, MYH10, TNNI1, TNNT2, and HAND1 and/or the posterior region comprises upregulation or downregulation of one of more genes selected from a list comprising CDX2 and CDX1.

In one embodiment, the posterior region comprises markers for neuromesodermal progenitors, wherein the markers for neuromesodermal progenitors are one or more genes selected from a list comprising TBXT, SOX2, NKX1.2, HOXB8, and HOXC9.

In one embodiment, the in vitro human embryo organoid comprises neural precursors, wherein the neural precursors are one or more genes selected from a list comprising SOX2, ZIC5, POU3F2, neural progenitors, wherein the neural progenitors are one or more genes selected from a list comprising ISI1, ZEB2, FOXC1, neural crest markers, wherein the neural crest are one or more genes selected from a list comprising FOXD3, S100B, HOXB9, SOX2, SOX10, CRABP1, TFAP2A, TFAP2B, neuron markers, wherein the neuron markers are one or more genes selected from a list comprising STMN2, ASCL1, ELAVL3, NEUROG1, and NEUROD4, neural tube markers, wherein the neural tube markers are one or more genes selected from a list comprising SOX3, PAX6, PAX7, and HES5, spinal cord markers, wherein the spinal cord markers are one or more genes selected from a list comprising OLIG3, SOX1, ZIC1, PAX7, and FGF8, and neural ectoderm markers, wherein the neural ectoderm markers are one or more genes selected from a list comprising SOX1 and PAX6.

In one embodiment, the in vitro human embryo organoid comprises one or more selected from a list comprising hematopoietic cells CD45⁺RUNX1⁺, RUNX1⁺CD45⁻, and CD45⁺RUNX1⁻, CD31⁺CD45⁻, CD31⁺CD45⁺, CD31⁻CD45⁺, CD31⁺CD34⁺CD45⁺, CD34⁺CD45⁻, CD34⁻CD45⁺.

In one embodiment, the hematopoietic cells further comprise one or more genes selected from a list comprising CD45, MYB, ITGA2B, SPN, GFI1B, SPINK2 and CD44.

In one embodiment, the hematopoietic cells further comprise markers for megakaryocytes, wherein the markers for megakaryocytes comprise one or more genes selected from a list comprising GP1BA, GP1BB and NFE2.

In one embodiment, the hematopoietic cells further comprise markers for macrophages, wherein the markers for macrophages comprise one or more genes selected from a list comprising CD68, CD86, MRC1 and TREM2

In one embodiment, the hematopoietic cells further comprise markers for eosinophil, basophil and mast cell progenitors, wherein the eosinophil, basophil and mast cell progenitors comprise one or more genes selected from a list comprising GATA2, IL1B, MYC, STAT5A and SOX4.

In one embodiment, the hematopoietic cells further comprise one or more genes selected from a list comprising lymphocytes CD52, CD69, HOPX, SPINK2, IL7R and NKG7.

In one embodiment, the in vitro human embryo organoid expresses one or more markers indicative of primordial germ cell-like cells selected from a group comprising NANOS3, POU5F1, PDPN, TFAP2C, PRDM1, PRDM14, CD38 and NANOG.

In one embodiment, the in vitro human embryo organoid expresses one or more markers indicative of neuronal cells selected from a group comprising S100B, FOXD3, SOX10, ELAVL3, NEUROD4, NEUROG1, SOX2, SOX10, CRABP1, TFAP2A, TFAP2B, STMN2, ASCL1, ELAVL3, NEUROG1, NEUROD4, SOX3, PAX6, PAX7, HES5, OLIG3, SOX1, ZIC1, PAX7, and FGF8.

In one embodiment, wherein the anterior region expresses TNNT2 and MYH2 genes.

In one embodiment, the in vitro human embryo organoid comprises contractile cardiomyocytes, wherein the markers for cardiomyocytes are one or more genes selected from a list comprising NKX2-5 and MYH2, TNNI1, ACTC1, TNNT2, MYH6, MYL7 and IRX3.

In one embodiment, the in vitro human embryo organoid comprises amnion cells, wherein the markers for amnion cells are one or more genes selected from a list comprising GABRP, VTCN1, WNT6 and TFAP2A,

In one embodiment, the in vitro human embryo organoid comprises mesenchyme cells, wherein the markers for mesenchyme cells are one or more genes selected from a list comprising PCOLCE, COL1A2, COL6A3 and HAND1.

In one embodiment, the in vitro human embryo organoid comprises hepatocytes, wherein the markers for hepatocytes are one or more genes selected from a list comprising AFP, ALB and CEBPA.

In one embodiment, the in vitro human embryo organoid comprises blood progenitors, wherein the markers for blood progenitors are one or more genes selected from a list comprising PECAM1/CD31, CD34, CDH5, RUNX1, PTPRC/CD45 and CD44.

In one embodiment, the in vitro human embryo organoid comprises splanchnic mesoderm, wherein the markers for splanchnic mesoderm are one or more genes selected from a list comprising WT1, LHX2 and GATA4,

In one embodiment, the in vitro human embryo organoid comprises paraxial mesoderm, wherein the markers for paraxial mesoderm are one or more genes selected from a list comprising MEOX1, SIX4 and FOXC2.

In one embodiment, the in vitro human embryo organoid comprises erythroid cells, wherein the markers for erythroid cells are one or more genes selected from a list comprising GATA1, KLF1, HBA1, HBA2, HBE1, HBE2, HBG1 and HBG2,

In one embodiment, the in vitro human embryo organoid comprises endothelial cells, wherein the markers for endothelial cells are one or more genes selected from a list comprising CD34, CD31, CDH5, KDR, SOX7, THY1, ITGB1, CXCR4, NRARP, GJA4, IGFBP4, VEGF1, FLT1, TMEM88, TPM1, PRTG, HOXB7, MEIS2, RNF19A, CAV1, REN, BNIP3, STC1 and FGF23.

In one embodiment, the in vitro human embryo organoid comprises hepatocytes (foetal liver progenitors), wherein the markers for hepatocytes are one or more genes selected from a list comprising AFP, APOA1, APOB, ALB and TF.

In one embodiment, the in vitro human embryo organoid comprises musculoskeletal precursors, wherein the musculoskeletal precursors are one or more genes selected from a list comprising NEFM, SIX1 and TNNT1.

In one embodiment, the in vitro human embryo organoid comprises hindgut, wherein the markers for hindgut are one or more genes selected from a list comprising CDX2, SOX2, SOX17, SHH and EPCAM.

In one embodiment, the in vitro human embryo organoid is derived in vitro from one or more human embryonic stem cells (ESCs).

In one embodiment, the in vitro human embryo organoid is derived in vitro from one or more human induced pluripotent stem cells (iPSCs).

According to a fourth aspect of the invention, there is provided an in vitro human embryo organoid at a post-gastrulation stage of development obtainable by a method according to the second or third aspects of the invention.

According to a fifth aspect of the invention, there is provided a progenitor cell or derivative thereof obtainable by the method according to the second or third aspects of the invention.

In one embodiment, the progenitor cell or derivative thereof is an endodermal cell, mesodermal cell, ectodermal cell, neural crest cell, neuronal progenitor, neuronal precursor cell, hematopoietic cell, endothelial cell, hepatocyte cardiomyocyte, HSPC, megakaryocyte, macrophage, eosinophil, basophil, mast cell, lymphocyte, primordial germ cell-like cell, amnion cell, mesenchyme cell, hepatocytes, foetal liver progenitor, blood progenitor, erythrocyte, myeloid cell, endothelial cell, musculoskeletal precursor cell, ESC and/or iPSC.

According to a sixth aspect of the invention, there is provided a kit comprising an in vitro human embryo organoid at a post-gastrulation stage of development according to the first aspect of the invention.

According to a seventh aspect of the invention, there is provided a system for drug screening for teratogenic and therapeutic purposes comprising an in vitro human embryo organoid at a post-gastrulation stage of development according to the first aspect of the invention.

The invention is described in the following non-limiting figures and tables.

### FIGURES

FIG. 1: Self-organization of hESCs into hEOs with three germ layers.

A, Experimental plan showing the generation of hEOs. Ch, Chiron (CHIR99021); Act, Activin A; E6, Essential 6 Medium; E8, Essential 8 Medium; RB27, Advanced RPMI medium supplemented with B27; hEOs, human Embryonic Organoids. B, Immunofluorescence staining of transiently induced cells showing pluripotent markers; POU5F1 (OCT4), SOX2 and NANOG (top); Primitive Streak markers, EOMES, SOX17 and TBXT (or BRACHYUARY) (bottom); GATA3 and pSMAD1/5/8 are negative (middle). Scale bar, 50 μm. C, Formation of the three germ layer derivatives on hEO from D2 to D8 derived from RUES2-GLR hESCs. Reporter colors represent ectoderm (SOX2-mCitrine), mesoderm (TBXT-mCerulean), and endoderm (SOX17-tdTomato). Scale bar, 250 μm. D, Schematic diagram showing the expression of three germ layer markers in hEOs. E-F, Representative confocal micrographs showing the primary germ layer derivatives at D4 on hEO sections derived from W15-tdTomato hESCs stained for SOX17, SOX2 and BRA (or TBX7). Scale bar, 200 μm (E), 100 μm (F).

FIG. 2: Self-organization of hESCs into embryo organoids (hEOs) establishes the three germ layer derivatives.

A, Immunofluorescence staining of W15 hES cells showing the expression of pluripotency markers. Top, POU5F1 (or OCT4), NANOG and SOX2; Middle, POU5F1, and SOX2; Bottom, SOX2. TBXT, EOMES and SOX17 are used as negative marker controls. Scale bar, 50 μm. B, Microscopic images showing the effect of Wnt agonist, Chiron, on hEO formation. Scale bar, 100 μm. C, Microscopic images showing the formation of hEO aggregates at 3 h and 24 h. Scale bar, 200 μm. D, Establishment of the three germ layer derivatives on hEO derived from RUES2-GLR hESCs. Reporter colors represent ectoderm (SOX2-mCitrine), mesoderm (TBXT-mCerulean), and endoderm (SOX17-tdTomato). Scale bar, 100 μm.

FIG. 3: Transcriptional characterization of hEOs at single cell resolution.

A, Experimental plan showing the generation 10× single-cell RNA sequencing (scRNAseq) data from hEOs at different time points. Twenty hEO were pooled for each stage, and scRNAseq was performed in replicates obtained from biologically independent experiments for each sampled time. B, UMAP showing the clustering and annotation of cell types at Day (D) 0, D2, D3, D4 and D8 obtained by 10×scRNAseq. C, Proportion of cells in each cluster along the progression of hEO from D0 to D8. D, Prediction score based on the mapping of cell types in CS7 human gastrula and hEOs at different time points. D-F, Co-embedding in a UMAP plot of the single-cell transcriptomes from a CS7 human gastrula and D3 hEOs. G, Heatmap comparing the transcriptome of D2-D4 hEOs with previously described human gastruloids. H, Heatmap comparing the transcriptome of CS11 NHP embryos to D8 hEOs. PS, primitive streak; Endo, Endoderm; Ecto, Ectoderm; EM, Early Mesoderm; PM, Paraxial Mesoderm; IM, Intermediate Mesoderm; MM, Mixed Mesoderm; AxM, Axial Mesoderm; YSM, Yolk Sac Mesoderm; EmM, Emergent Mesoderm; NM, Nascent Mesoderm; Epi, Epiblast; Ery, Erythrocytes; HEP, Hemogenic Endothelial Progenitors.

FIG. 4: Quality control of scRNAseq data.

Metrics used to assess the quality of the scRNA libraries across all samples and clusters. 10× Genomics was performed in replicates derived from independent experiments. For each replicate, 20 hEO were pooled together and loaded into the 10×-Genomics Chromium using the single cell 3′ reagents kit v3. From left to right the plots show the fraction of reads mapped to mitochondrial genome, unique molecular identifier counts (or transcripts) per cell, fraction of reads mapped to ribosomal RNA and number of detected genes.

FIG. 5: Transcriptional characterization of hEOs at the level of single cell resolution.

A, Uniform manifold, approximation and projection (UMAP) of all cells computed from genes with highly variable expression across all samples represented in UMAP plot after having performed batch correction with Scanorama (see methods) with all annotated clusters, and B, across all time points, D0, D2, D3, D4 and D8. C, UMAP at D0 showing key markers of PS D, UMAP at D0 showing key markers of Ectoderm E, UMAP at D0 showing key markers of APS F, UMAP at D0 showing key markers of PPS and G, UMAP at D0 showing key markers of pluripotency. D, day. PS, primitive streak. APS, anterior PS. PPS, posterior PS. Bad quality clusters are also shown in panels C-G.

FIG. 6: Transcriptional profiles showing the expression of primitive streak (PS) and mesoderm derivatives in the hEOs.

A, Box plot showing the expression of key markers of PS, including TBXT, MIXL1, WNT3, EVX1 and NKX1-2 in hEO. B, Diffusion component (DC) analysis of PS representing key marker expression including TBXT, MIXL1, NKX1-2, EVX1 and FGF2. C, Box plots showing the expression of key markers of mesodermal derivatives, including early mesoderm, intermediate mesoderm, paraxial mesoderm, somitic mesoderm and cardiac mesoderm in hEO. D, DC analysis of mesoderm derivatives representing key marker expression, including MESP1, MESP2, BMP4 and FGF2.

FIG. 7: Transcriptional profiles showing the expression of endoderm and ectoderm derivatives in the hEOs.

A, Diffusion component (DC) analysis of Endoderm representing key marker expression including SOX17, FOXA2, MNX1, EPCAM and APOA1. B, Box plot showing the expression of key markers of ectodermal derivatives, including neurons, neural tube and neural crest in hEO. C, DC analysis of Ectoderm derivatives representing key marker expression, including ASCL1, PAX6, OLIG3, CRABP1, ELAVL3, NEUROG1 and STMN2.

FIG. 8: Comparison of hEOs with CS7 human gastrula and human gastruloids.

A, Prediction score based on the mapping of cell types in CS7 human gastrula and hEOs at different time points. B, UMAP showing the expression of key ExE-mes markers in D2, D3 and D4 hEOs, mostly in the mixed mesoderm cluster. Bad quality clusters are also shown. C, Diffusion map displaying the trajectory of differentially expressed genes in the PS and NM in CS7 human gastrula and D3 hEOs. D-E, Integration of D2, D3 and D4 hEOs transcriptome dataset with previously described (D) human gastruloid-1 and (E) human gastruloid-2. F, Heatmap comparing the transcriptome of hEOs with human gastruloid-2 dataset. Ecto, Ectoderm; Endo, Endoderm; MM, mixed mesoderm; IM, intermediate mesoderm; PS, primitive streak; EM, early mesoderm; PM, paraxial mesoderm; NC, neural crest; Neur, neurons, SM, somatic mesoderm; CM, cardiac mesoderm; AM, advanced mesoderm; CMy, cardiomyocytes; ExM, extra-embryonic mesenchyme; NMP, neuromesodermal progenitors.

FIG. 9: Comparison of hEOs with cynomolgus monkey and rostral-caudal axis formation.

A, UMAP Comparing the transcriptome of CS8-11 non-human primate (NHP) embryos to D3-8 hEOs. B, D2 hEO sections showing an elongation along the rostral-caudal axis represented by the expression of GATA6 and CDX2 (top) and GATA6 and TEXT (bottom), respectively. Scale bar, 100 μm. C, UMAP of D2, D3, D4 and D8 hEOs showing anterior (GATA6) and posterior markers (CDX2 and TBXT). D-F, Fraction of cells showing differential gene expression in mixed mesoderm and intermediate mesoderm of D3 hEOs as measured against rostral versus caudal gene expression in advanced mesoderm cluster of CS7 human gastrula. E, Diffusion map showing the separation of rostral and caudal genes present in (F) mixed mesoderm (MM) and intermediate mesoderm (IM) of D3 hEOs. PS, primitive streak; NM, nascent mesoderm; EM, early mesoderm.

FIG. 10: Human embryo organoids reveal the emergence of neuromesodermal progenitors (NMP).

A, Heatmap showing the comparison of rostral and caudal genes in the advanced mesoderm of CS7 gastrula versus Intermediate and mixed mesoderm clusters in D3 hEOs. B, Volcano plot with rostral and caudal genes expressed in D3 hEOs analysed in (A). C, BOX plot showing the expression of key NMP markers in scRNA dataset. D, Live microscopic images of D3 hEOs generated from RUES2-GLR reporter hES cell line showing the co-expression of SOX2 and TBXT. E, Confocal micrographs of IF showing NMP in D3 hEOs generated from RUES2-GLR (left) and W15 hESCs (right), respectively. Cells (nuclei) co-expressing SOX2 and TBXT are circled. Outlined regions are magnified below the corresponding image. Scale bar, 50 μm. F, UMAP of D2, D3 and D4 hEOs with co-expression of key NMP markers TBXT, SOX2 and NKX1-2. Schematic diagram shows the localization of NMP in the posterior region of the hEOs. NMP, neuromesodermal progenitors.

FIG. 11: Human embryo organoids display the emergence of NMP.

A-C, Dot plots showing the expression of key markers of neural crest and neurons in D4 and D8 hEOs, and spinal cord markers in D8 hEOs. UMAPs showing the expression of D, F, Neural Crest and E, G, Neuronal markers on D4 and D8 hEOs, respectively. In panels D-G bad quality clusters are also shown.

FIG. 12: human embryo organoids display the emergence of neuronal lineage.

A-B, UMAPs showing the expression of key markers of (A) Neural tube and (B) Spinal cord on D8 hEOs. Bad quality clusters are also shown. C-E, Confocal micrographs of IF showing the emergence of neural ectoderm (SOX1 and PAX6) and neural crest (SOX2, TFAP2A) on D8 hEO sections, respectively. Scale bar, 100 μm (C), 100 μm (D) 50 μm (E). F, Waddington OT analysis showing the network of neuronal precursors on D8 linked to NMP and ectoderm at previous timepoints (see method).

FIG. 13: Detection of hPGCLC in the absence of exogenous BMP supplementation in hEO.

A, Microscopic images showing the effect of Wnt agonist, Chiron, for the specification of hPGCLCs, as shown by the expression of NANOS3 on D3 hEOs. B, Microscopic images showing the detection of hPGCLCs in the absence of external BMP, as shown by NANOS3 reporter in hEOs at different time points. C-D, Confocal micrographs showing the expression of key hPGCLC markers, TFAP2C, POU5F1 (or OCT4), SOX17 and NANOS3 (RFP), SOX17, TFAP2C in hEO sections on D4 and D8, respectively. E, Sub-clustering and reintegration of PS, EM, Ectoderm and NMP clusters from D2 and D3. PS, primitive streak; EM, early mesoderm; NMP, neuromesodermal progenitors; AMLC, amnion like cells. F, Diffusion components (DC) analysis showing the expression of TFAP2A and TFAP2C on D2 hPGCLCs, (G) D3 hPGCLCs and (H) D2 and D3 combined.

FIG. 14: Detection of hPGCLC in the absence of exogenous BMP supplementation in hEOs.

A, Microscopic images showing the expression of NANOS3 reporter in hEO on D2, D3, D4 and D8 without addition of BMP in the culture medium. Scale bar, 50 μm. B-C, Representative confocal micrographs showing key hPGCLC markers (NANOS3 (RFP), SOX17, TFAP2C and POU5F1 (or OCT4) by IF in hEO sections on D2 and D3 respectively. D, Schematic diagram showing the potential location of PGCLCs in hEOs. E, UMAP showing the expression of hPGCLCs in the PS and AMLC during early specification (D2 and D3). F, Dot plot with key PGCLC markers expressed in hEOs, and the enrichment of BMP4 in AMLC. G, Confocal micrographs showing the co-expression of amnion like cells (AMLC) marker, ISL1 and PGCLC identifier SOX17. Scale bar, 100 μm. H, Microscopic images showing the Nanos3-tdTomato reporter in the hEOs cultured in presence of BMP inhibitor LDN193 at different time points. Scale bar, 100 μm.

FIG. 15: Post-gastrulation development of hEOs reveal beating heart tube and haematopoietic cells.

A, Schematics of extended culture system of hEOs recapitulating gastrulation and post-gastrulation development of the human embryo. B, Bright-field images of D8 hEO cultured in rotary culture with 1 μM SB and 10% Matrigel. Scale bar, 100 μm. C, Bright-field images of D8 hEO cultured in rotary culture with 1 μM SB without Matrigel. Scale bar, 100 μm. D, Frequency of beating hEO (N=4 separate experiments, n=170-250 hEO per condition). E, Sectional view of the primitive heart tube expressing cardiac troponin type 2 (surface marker). Nuclear staining is shown in greyscale. Scale bar, 100 μm. Outlined region shows the magnified heart tube (right). Scale bar, 25 μm. F, Sectional view of the primitive heart tube expressing myosin heavy chain 2 (MYH2, surface marker) in D8 hEOs. Nuclear staining is shown in greyscale. Scale bar, 100 μm. Outlined region shows the magnified heart tube (MYH2⁺) (right and bottom panels). Scale bar, 50 μm. G, Frequency of elongated hEO after 4 days of static culture with or without 1 μm SB43 (N=4 experiments, n=750-800 hEO; unpaired student t test p_{adj}=0.0016) H, Frequency of beating hEO (N=4 separate experiments, n=170-250 hEO per condition, One Way ANOVA with Tukey's multiple comparisons all p_{adj}<0.0001). I, Bright-field images of D14 hEOs with visible red inclusions. Scale bar, 250 μm. Outlined image is magnified on the right panel. Scale bar, 100 μm. J, Frequency of hEO with red inclusions (N=4 separate experiments, n=170-250 hEO per condition, One Way ANOVA with Tukey's multiple comparisons p_{adj}<0.0001, −SB/−SB vs. +SB/+SB not significant). K, Confocal micrographs from wholemount IF staining with SOX17 and RUNX1+ haemogenic progenitors. Scale bar, 50 μm. Outlined region is shown in the magnified image of the right top and bottom panel. Scale bar, 50 μm. L, IF staining of D14 hEOs showing the expression of haemogenic progenitors (RUNX1⁺) and hematopoietic cells (CD45⁺). Scale bar, 100 μm. Outlined regions are shown on the magnified images. Scale bar, 20 μm. M, Flow cytometry plots of CD45 expression in red hEO or in clear hEO, pre-gated on live, single cells based on a CD45 FMO. N, Frequency of live CD45⁺ cells in control vs. red hEO relating to FIG. 4L (unpaired student t-test, p=0.0015, N=3 replicates of 5-10 hEOs). O, Representative histogram overlays of CD45− (gray) and CD45+ (red) cells from red hEO, showing expression of CD31, c-KIT, and EpCAM in both populations normalized to mode, and correlating expression of CD31 and CD45⁺ cells within the entire population of red hEOs. Top right figure: gray, CD45⁻CD31⁻; purple, CD31⁺CD45⁻; turquoise, CD31⁺CD45⁺; green, CD45⁺CD31⁻. Dotted arrow shows the potential transition from endothelial to hematopoietic cells.

FIG. 16: Post-gastrulation development of human embryo organoids.

A, Wholemount IF staining shows the expression of derivatives of the three germ layers (maximum intensity projection), representing ectoderm (SOX2), mesoderm (TBXT) and endoderm (SOX17) on D2 hEOs. Scale bar, 50 μm. B, D4 hEOs shows the expression of key PGC marker, NANOS3, in a W15-NANOS3-tdTomato reporter hES cell line. Scale bar, 250 μm. C, Confocal micrographs showing the maximum intensity projection of three germ layers, as an expression of SOX17 (representing endoderm), SOX2 (representing ectoderm) and TBXT (representing mesoderm). Scale bar, 50 μm. D, Bright field image and reporter overlay of D8 hEO cultured in rotary culture which failed to progress (no SB). Scale bar, 500 μm. E, Brightfield images of hEO cultured in static culture with Collagen 1 (D6), and in rotary culture with HCS or EUCM2 (D4+). Scale bars 500 μm). F-G, Microscopic images showing the expression of SOX17 (red), SOX2 (green) and BRA (blue) in hEOs developed with (F) or without (G) Matrigel in rotary culture system. Scale bar, 100 μm. H, D8 hEO showing the initiation of gut tube formation. Left panel, BF image. Right panel, SOX17+ gut tube with SOX2 expression at one pole. Scale bar, 250 μm. I, Outlined region represents the magnified image of neuomesodermal progenitors (NMP) (right) as a co-expression of SOX2 and TBXT. Scale bar, 25 μm. J, Confocal micrographs of the wholemount IF staining with SOX9⁺ neural crest cells in D8 hEOs. Scale bar, 100 μm. K, BF image of hEO cultured in SB on D4, D8, and D21, showing doubling of hEO size over duration of the three weeks in culture. Consistent scale bar 100 μM.

FIG. 17: Post-gastrulation development of human embryo organoids.

A, IF staining of D14 hEOs showing the expression of haemogenic progenitors (RUNX1⁺) and haematopoietic cells (CD45⁺). Scale bar, 50 μm. B, Microscopic images showing the expression of haematopoietic (erythroid) cells in D22 hEOs. Scale bar, 500 μm. C, Flow cytometry analysis of hEOs with (red) or without (clear) HPCs in triplicates. HPCs, haematopoietic cells.

FIG. 18: Non-integrated stem cell-based embryo models (niSTEMBRYOs) display multilineage organogenesis.

A, Schematics showing the experimental plan to derive multilineage organogenesis model. Scale bar, Day 0, Day 2, Day 3 and Day 4, 100 μm; Day 1, 50 μm. B, Day (D) 2 aggregates displaying the expression of three germ layer derivative markers; SOX2 (ectoderm), SOX17 (endoderm) and TEXT (or BRACHYURY) (mesoderm). Scale bar, 50 μm. C, D4 aggregates showing the expression of primary germ layers (SOX2, SOX17) and amnion-like cells (TFAP2A) (AMLC). Scale bar, 50 μm. D, D4, D8 and D14 niSTEMBRYOs showing the increment in size during development using static and kinetic rotary culture. Scale bar, D4 and D8, 100 μm. D14, 500 μm. E, Quantitative expression of contracting cardiomyocytes observed at different culture conditions in D8 niSTEMBRYOs. SB43+CH, niSTEMBRYOs cultured with TGF-b inhibitor SB43 from D4 supplemented with 0.25 μM Chiron. CH, niSTEMBRYOs cultured with 0.25 μM. HCS, niSTEMBRYOs cultured with human cord serum (HCS) supplemented with rat embryo serum. P<0.0001. F, Wholemount immunofluorescence (IF) staining of D8 niSTEMBRYOs showing the expression of cardiomyocytes markers, NKX2-5 and myosin heavy chain (MYH)-2. Scale bar, 100 μm. Region in the inset is magnified and shown on the lower panel. Scale bar, 50 μm. G, niSTEMBRYO showing the appearance of red pigmentation at D14, indicating hematopoietic cells. Scale bar, 100 μm. H, Quantitative expression of niSTEMBRYOs displaying hematopoiesis (red pigmentation) at D14. (N=174 samples, 5 experiments). p<0.0001. SB43+CH, niSTEMBRYOs cultured with TGF-b inhibitor SB43 from D4 supplemented with 0.25 μM Chiron. CH, niSTEMBRYOs cultured with 0.25 μM. HCS, niSTEMBRYOs cultured with human cord serum supplemented with rat embryo serum. I-J, Quantitative flow cytometry analysis showing the expression of CD45⁺ hematopoietic cells (HCs) in D14 niSTEMBRYOs. Red, niSTEMBRYOs with red pigmentation. Control, clear niSTEMBRYOs without red pigmentation. K, Flow cytometry analysis showing the transition of endothelial to hematopoietic cells. Distinct population of endothelial cells (ECs) and hematopoietic cells (HCs) are shown in different colors. Singly positive ECs cells are CD31⁺45⁻ whereas bona fide HCs are CD45⁺31⁻ with a double positive intermediary, CD31⁺CD45⁺. Grey, CD45⁻ population. Purple, CD31^{low} population. Red, CD31^{high} population. Blue, CD31⁺CD45⁺ intermediary population. Green, CD45⁺ differentiated hematopoietic cells.

FIG. 19: Generation of non-integrated stem cell-based embryo models (niSTEMBRYOs) from human embryonic stem cells (ESCs).

A, Immunofluorescence (IF) staining showing the expression of pluripotency (OCT4 or POU5F1, SOX2 and NANOG) and differentiation marker (BRACHYURY or TBXT, SOX17 and EOMES) at day (D) 0, that is, 24 h prior to aggregates formation. Scale bar, 100 μm. B, Still image of the rotary culture system used to generate niSTEMBRYOs. Different parts of the rotary culture system are shown in the figure labels. C, Wholemount IF staining showing the expression of primary germ layers (SOX2, SOX17 and TBXT) in D4 aggregates. Scale bar, 100 μm. Region in the inset is enlarged on the right-hand side. Scale bar, 50 μm. D, Microscopic images showing the disorganized structure of aggregates cultured with CH only during kinetic culture. Scale bar, 250 μm. E-H, Microscopic images of 3D aggregates cultured with (E) Collagen Type I, (F) human cord serum (HCS), (G) HCS supplemented with TGF-ß inhibitor, SB43, and (H) 10% Matrigel®. Scale bar, 500 μm (E-G); 100 μm (H).

FIG. 20: Transcriptome characterization of niSTEMBRYOs.

A, Isolation of red and non-red aggregates at D14 for quantitative flow cytometry analysis. Samples with visible red pigmentation were labeled as Red, whereas those that were not visibly red were labeled as non-red or clear. Cells in the inset are enlarged on the left- and right-hand sides. B-C, Quantitative flow cytometric measurement showing the frequency and intensity of endothelial (CD31, CD34, c-KIT) and hematopoietic (CD45) cells. D-E, Pie charts showing the percentages of each cell type detected in (D) D8 and (E) D14 niSTEMBRYOs. F-G, Dot plots showing the key markers and cell types detected to identify cell clusters in niSTEMBRYOs from (F) D8 and (G) D14. Colors represent the scaled expression and size encodes the proportion of gene-expressing cells.

FIG. 21: niSTEMBRYOs reveal embryonic hematopoiesis.

A, Uniform manifold approximation projection (UMAP) plot showing the cell types and clusters obtained from 10× single cell RNA sequencing (scRNAseq) of niSTEMBRYOs, sampled at D8 and D14. Color codes for each cluster are shown on the right. B, Dot plots showing the key markers detected by scRNAseq at D8 and D14 niSTEMBRYOs as shown in (A). Colors represent the scaled expression and size encodes the proportion of gene-expressing cells. C, Heatmap projected by comparing transcriptome profile of D14 niSTEMBRYOs with that of Carnegie stage (CS) 12-CS16 human embryos. X-axis, in vivo cell types obtained from Xu et al., 2023; Y-axis, D14 niSTEMBRYO cell types. D, UMAP plot showing endothelial (EC), hematopoietic (HC) and erythroids cell types obtained from D8 and D14 niSTEMBRYOs. D8 and D14 ECs are clustered together, whereas D14 HCs and erythroids are separate. E, UMAP visualization of the expression of curated feature genes for the identification of endothelial (CD34⁺CD31⁺), hematopoietic (CD45⁺MYB⁺) and erythropoietic cells (HBA1⁺HBE1⁺). HBA1 and HBE1 are fetal hemoglobin genes. F, Sankey plot showing the alignment of blood cell types obtained from D14 niSTEMBRYOs by single cell RNA sequencing with that of the blood cell types from CS12-CS16 human embryos. Left column, niSTEMBRYOs blood cell types; right column, predicted in vivo blood cell types from CS12-CS16 human embryos. G, UMAP plot showing the representative blood cell subtypes detected in D14 niSTEMBRYOs as shown in (F). H, Dot plots showing the key markers associated with blood cell subtypes detected in D14 niSTEMBRYOs obtained by comparing with in vivo CS12-CS16 human embryo dataset (Xu et al., 2023). Key blood cell types include megakaryocytes, macrophages, s, hematopoietic stem and progenitor cells (HSPCs), lymphocytes and eosinophil/basophil/mast cell progenitors. Colors represent the scaled expression and size encodes the proportion of gene-expressing cells.

FIG. 22: Transcriptome profiles of niSTEMBRYOs correspond to CS12-CS16 human embryos.

A, Heatmap comparing the transcriptome profile of CS12-CS16 human embryos (Xu et al., 2023) with that of D14 niSTEMBRYOs obtained from 10× single cell RNA sequencing. Color code for each cell type is shown next to the UMAPs. B, Sankey plot showing the resemblance between cardiomyocytes detected in D14 niSTEMBRYOs with cardiomyocytes detected in CS12-CS16 embryonic heart (Xu et al., 2023). C, UMAP visualization of the expression of curated feature genes for the identification of endothelial (KDR or FLK1), hematopoietic (ITGA2B, SPINK2, CD44, SPN) and erythropoietic cells (HBG1, HBG2, HBA2, KLF1, GATA1) in D14 niSTEMBRYOS.

FIG. 23: niSTEMBRYOs cultured with SB from D1 reveal cardiomyocytes and hindgut.

A, Experimental plan showing the generation of niSTEMBRYOs cultured with TGF-ß inhibitor, SB43 from D1 (niSTEMBRYOs:SB^{early}). B, Wholemount IF staining showing the expression of ectoderm (SOX2⁺) and mesoderm (TBXT⁺ or BRACHYURY) derivatives as well as amnion like cells (TFAP2A⁺) in D2 aggregates (SB^{early}). Scale bar, 50 μm. C, Microscopic images showing the formation of gut tube with the expression of SOX17 and SOX2 in D8-niSTEMBRYOs:SB^{early}. Scale bar, 200 μm. D, Microscopic (bright-field) image of a D14 niSTEMBRYO obtained after static and kinetic culture cultured with TGF-ß inhibitor, SB43 from D1 (D14 niSTEMBRYOs:SB^{early}). Scale bar, 500 μm. E, Wholemount IF staining showing the expression of cardiac troponin type 2 (TNNT2) in D8 niSTEMBRYOs cultured with SB43 from D1 (niSTEMBRYOs:SB^{early}). Scale bar, 100 μm. F, Wholemount IF staining showing the SOX17⁺SOX2⁺ hindgut in D8-niSTEMBRYOs:SB^{early}. Scale bar, 100 μm. G, Quantitative expression of contracting cardiomyocytes observed in D8 niSTEMBRYOs:SB^{early} and D8 niSTEMBRYOs:SB^{late}. No significant difference was observed between the two groups. H-I, Pie charts showing the cell types identified by transcriptome profile of (H) D8 and (I) D14 niSTEMBRYOs cultured with SB43 from D1 (D8-niSTEMBRYOs:SB^{early}).

FIG. 24: Early inhibition of TGF-ß signaling inhibits hematopoietic induction in niSTEMBRYOs.

A, D2 aggregates showing the expression of three germ layer derivatives, including mesoderm (TBXT⁺ or BRACHYUARY), ectoderm (SOX2⁺) and endoderm (SOX17⁺) cultured in presence of TGF-b signaling inhibitor SB43 from D1 (referred to as niSTEMBRYOs:SB^{early}). Scale bar, 50 μm. B, Wholemount IF staining showing the loss of TBXT in D4 aggregates, while SOX2 (ectoderm derivative) and SOX17 (endoderm derivative) are expressed upon early inhibition of TGF-ß pathway with SB43 (niSTEMBRYOs:SB^{early}). Scale bar, 100 μm. C, UMAP showing the cell types and clusters obtained from single cell RNA sequencing (scRNAseq) of niSTEMBRYOs cultured with SB43 from D1 (niSTEMBRYOs:SB^{early}), sampled at D8 and D14. Color codes for each cluster are shown on the left. D, Dot plots showing the key markers and cell types detected by scRNAseq at D8 and D14 niSTEMBRYOs:SB^{early}, as shown in (C). Colors represent the scaled expression and size encodes the proportion of gene-expressing cells. E-F, Differential gene expression (DGE) showing highly enriched genes among endothelial cells (ECs) between niSTEMBRYOs cultured in SB43 from D1 (niSTEMBRYOs:SB^{early}) and niSTEMBRYOs cultured in SB43 from D4 (niSTEMBRYOs:SB^{late}) at (E) D8 and (F) D14. G, Volcano plot showing the upregulation of secreted ligands such as Stanniocalcin-1 (STC1) and fibroblast growth factor (FGF) 23 in D14 niSTEMBRYOs:SB^{early}, thus inhibiting hematopoietic induction in D14 niSTEMBRYOs:Sb^{early} as shown in (H). STC1 and FGF23 are downregulated in D14 niSTEMBRYOs:SB^{late}, thus promoting hematopoietic induction. H, Quantitative expression of the aggregates showing hematopoietic cells (HCs) in D14 niSTEMBRYOs:SB^{late} and D14 niSTEMBRYOs:SB^{early} as measured by visible red pigmentation. (N=196/1170; 5 experiments). No red pigmentation was observed in D14 niSTEMBRYOs:SB^{early}. P<0.0001.

FIG. 25: Transcriptome profiles of niSTEMBRYOs:SB^{early} correspond to CS12-CS16 human embryos.

A-B, Dot plots showing cell clusters and population identified at D8 and D14 in niSTEMBRYOs cultured with TGF-ß inhibitor, SB43 from D1 (SB43^{early}). Colors represent the scaled expression and size encodes the proportion of gene-expressing cells. C-D, UMAP plots comparing the transcriptome profile of (C) D14 niSTEMBRYOs:SB^{early} with (D) CS12-CS16 human embryos (Xu et al., 2023). Key common cell types are encircled. Color codes show the identified cell types at the left-hand side. N/A refers to the cell types absent in the shown dataset (C) that are present in the corresponding compared dataset (D) and vice versa. E, Heatmap projection showing the correlation of transcriptome profile between D14 niSTEMBRYOs (SB43^{early}) with that of Carnegie stage (CS) 12-CS16 human embryos (Xu et al., 2023). X-axis, in vivo cell types; Y-axis, D14 niSTEMBRYO (SB43^{early}) cell types.

FIG. 26: niSTEMBRYOs cultured with SB43 early and late are transcriptionally different.

A-B, Integrated bar plots showing the comparative transcriptome features of cell types identified in (A) D8 and (B) D14 niSTEMBRYOs cultured with SB43 from day 1 (SB43^{early}) and niSTEMBRYOs cultured with SB43 from day 4 (SB43^{late}). Red bar, SB^{early}. Blue bar, SB^{late}. C, Major gene ontology (GO) terms identified on the basis of differential gene expression (DGE) enrichment in endothelial cells (ECs) (SB43^{late} vs SB43^{early} niSTEMBRYOs) of D8 niSTEMBRYOs. D, Violin plots showing the upregulation of key markers associated with hematopoiesis, such as FLT1 and ENG in the endothelial cells (Ecs) of D8 niSTEMBRYOs:SB^{late}, thus promoting hematopoietic induction. MYCT1 and MECOM are downregulated in the ECs of D8 niSTEMBRYOs:SB^{early}, thus failing to induce hematopoietic cells. E-F, Major gene ontology (GO) terms identified on the basis of differential gene expression (DGE) enrichment in the endothelial cells of (E) D14:SB^{late} niSTEMBRYOs and (F) D14:SB^{early} niSTEMBRYOs. G, Schematics showing D14 niSTEMBRYOs cultured with SB from D1 (early) and D4 (late). Key difference detected in each model is represented in the schematics.

FIG. 27: niSTEMBRYOs exhibit transition from hematopoietic stem and progenitors to hematopoietic cells.

A, UMAP plot showing the subclusters of hematopoietic cells (HCs) obtained from D14 niSTEMBRYOs:SB^{late}. HCs cluster is subclustered into Cluster 0, 1, 2 and 3. B, Pseudotime trajectories displaying the projected expression of key endothelial and hematopoietic markers, including RUNX1, CD34, PECAM1 (or CD31), PTPRC (or CD45), GATA1 and CDH5 in two branches, branch A and B, indicating transition from hematopoietic stem and progenitors (HSPCs) to hematopoietic cells (HCs). C, UMAP visualization showing the curated feature genes for the identification of transition from Hematopoietic stem and progenitor cells to hematopoietic cells. Hematopoietic stem and progenitor cells (HSPCs) cells show the detection of CDH5, CD34, SOX7 and SOX18. Hematopoietic cells (HCs) in branch A shows the detection of CD4, CD36, GJA5 and MRC1 and branch B shows the detection of CD9, ITGA2B, GFI1B and GATA1. D, UMAP plots showing the curated feature genes identifying the molecular signature of hematopoietic stem cells in niSTEMBRYOs cultured with SB from D4 (SB^{late}). Level of expression is shown on the right for each UMAP, and feature genes are encircled in the corresponding UMAPs. E, UMAP plots showing the curated genes featuring the transition from hematopoietic progenitors to erythro-myeloid progenitors in niSTEMBRYOS cultured with SB from D4 (SB^{late}). Detection of early hematopoietic cells expressing LIN28A and STXBP6 and definitive regulators of hematopoietic cells expressing EAF2, BCL11A and CX3CR1 are shown. Markers of transition to hematopoietic lineage, such as MYCN and KCNK17 as well as erythro-myeloid progenitors, MYCT1, CD15 (or FUT4), CD16 (or FCGR3A) are also shown.

FIG. 28: niSTEMBRYOs display transition from hematopoietic stem and progenitors to hematopoietic cells.

A, Developmental trajectory showing the transition of hematopoietic and stem progenitor cells (HSPCs) towards hematopoietic cells (HCs), as shown by bifurcation into two branches, branch A and B. B, Dot plots showing the expression of key markers detected in endothelial cells (ECs), hematopoietic stem and progenitor cells (HSPCs), hematopoietic cells (HCs) and erythroids in D14 niSTEMBRYOs cultured with SB from D4 (SB^{late}). Markers detected in endothelial cells (ECs) in D14 niSTEMBRYOs cultured with SB from D1 (SB^{early}) is also shown, which is similar to the ECs in SB^{late} niSTEMBRYOs. Colors represent the scaled expression and size encodes the proportion of gene-expressing cells. C, UMAP visualization of the expression of curated feature genes showing hematopoietic stem and progenitor (RUNX1) and hematopoietic marker (CD45 or PTPRC) in D14 niSTEMBRYOs:SB^{early} and D14 niSTEMBRYOs:SB^{late} D14 niSTEMBRYOs:SB^{early} lacked both of these markers, thus failing to induce hematopoietic cells. D, Wholemount IF staining of D14 niSTEMBRYOs showing the expression of hepatocytes and hematopoietic cells (HCs). Scale bar, 100 μm. Magnified images from the inset are shown on the right-hand side. Image on the right shows the expression of CD45⁺ (or PTPRC) hematopoietic cells interspersed between alfa fetoprotein (AFP⁺) potential hepatocytes. Scale bar, 25 μm. E, IF images showing the expression of bona fide hematopoietic marker, CD45 and hematopoietic progenitor marker, RUNX1 in D14 niSTEMBRYOs. Scale bar, 20 μm. CD45⁺ cells are potentially differentiated HCs; RUNX1⁺ cells are potentially hematopoietic stem and progenitor cells (HSPCs) and CD45⁺RUNX1⁺ cells are potentially transient cells undergoing EHT. F, Carnegie stage (CS) 15 human embryonic tissue showing the expression of CD45⁺ HCs and RUNX1⁺ hematopoietic progenitor cells in the mesonephros region. Scale bar, 500 μm. Image in the inset is magnified and shown at the bottom. Image at the bottom shows the expression of CD45⁺ HCs and RUNX1⁺ hematopoietic progenitor cells. HCs are CD45⁺RUNX1⁻ and CD45⁻RUNX1⁺. CD45⁺RUNX1⁺ double positive intermediary cells are indicated by white arrow, potentially indicating a transition from endothelial to hematopoietic cells. Scale bar, 20 μm. Arrow shows ventral (V), dorsal (D), cranial (Cra) and caudal (Cau) surface of the embryo section. Regions of mesonephros (Mn) and spinal cord (SC) are indicated. Head (H*), tail (T*) and other organs (such as lungs) are excised and donated for other studies.

FIG. 29: niSTEMBRYOs show hematopoietic induction similar to human embryos.

A, UMAP visualization of the expression of curated feature genes showing hematopoietic (MYB) and endothelial markers (CD34 and CD31/PECAM1) in D14 niSTEMBRYOs:SB^{early} and D14 niSTEMBRYOs:SB43^{late}. CD31 and CD34 were detected in endothelial cells of D14 niSTEMBRYOs:SB43^{early} and D14 niSTEMBRYOs:SB43^{late}. However, MYB, a marker of definitive hematopoiesis, was detected only in hematopoietic cells in D14 niSTEMBRYOs:SB43^{late}, promoting hematopoietic induction. B-C, Confocal micrographs of immunofluorescence staining showing the emergence of hepatocytes expressing alfa-fetoprotein (AFP) and albumin (ALB) in D14 niSTEMBRYOS. Scale bar, 50 μm. D, Immunofluorescence (IF) images of CS15 human embryonic tissue section showing hematopoietic cells (HCs) expressing CD45 and hematopoietic progenitor cells expressing RUNX1 in the mesonephros region. Scale bar, 200 μm. Mesonephros region in the inset is magnified and shown in the middle. Image in the middle shows the expression of CD45⁺ HCs, RUNX1⁺ hematopoietic progenitor cells and CD45⁺RUNX1⁺ double positive intermediary cells. Scale bar, 20 μm. CD45⁺RUNX1⁺ double positive intermediary cells in the middle image are magnified and shown on the right-hand side. Scale bar, 10 μm. Arrow shows ventral (V), dorsal (D), cranial (Cra) and caudal (Cau) surface of the embryo section. Mesonephros (Mn) region is indicated. E, Dot plots showing the key markers expressed in hematopoietic subclusters identified as hematopoietic stem and progenitor cells (HSPCs) (sub-cluster 0) and hematopoietic cells (HCs) (sub-lusters 1 and 2). Subcluster 3 also represents potential progenitor population. F, Dot plots showing the key markers expressed in endothelial, hematopoietic and erythroid cells of D8 and D14 niSTEMBRYOs (niSTEMBRYOs:SB^{late}). Key endothelial markers detected in D8 and D14 niSTEMBRYOs (niSTEMBRYOs:SB^{late}) are common to both time points. Colors represent the scaled expression and size encodes the proportion of gene-expressing cells in (B) and (C). G, UMAP plots showing that the curated feature genes of hematopoietic stem cell (HSC) signature is absent in niSTEMBRYOs cultured with SB from D1 (SB^{early}). Endothelial cells in D14 niSTEMBRYOs:SB^{early} are encircled and magnified to show the lack of expression of HSC identity.

### SPECIFIC DESCRIPTION

The present disclosure will now be further described. In the following passages, different aspects of the disclosure are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Generally, nomenclatures used in connection with, and techniques of, cell and tissue culture, pathology, oncology, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. See, e.g., Green and Sambrook et al., Molecular Cloning: A Laboratory Manual, 4th ed., Cold Spring Harbor Laboratory Press, Cold Spring Harbor, N.Y. (2012); Therapeutic Monoclonal Antibodies: From Bench to Clinic, Zhiqiang An (Editor), Wiley, (2009); and Antibody Engineering, 2nd Ed., Vols. 1 and 2, Ontermann and Duebel, eds., Springer-Verlag, Heidelberg (2010).

Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

According to a first aspect of the invention, there is provided, an in vitro human embryo organoid at a post-gastrulation stage of development comprising cells expressing markers characteristic of endodermal cells, cells expressing markers characteristic of mesodermal cells, cells expressing markers characteristic of ectodermal cells, and primordial germ cell-like cells (hPGCLCs).

The term an in vitro human embryo organoid at a post-gastrulation stage of development refers to an organoid which was created by inducing a change in cellular states from either pluripotent human embryonic stem cells (hESCs) or induced pluripotent stem cells (iPSC) to an in vitro human embryo organoid at a post-gastrulation stage of development comprising the three germ-layer derivatives and human primordial germ cell-like cells (hPGCLCs). In further embodiments, the in vitro human embryo organoid is derived in vitro from one or more stem cell selected from a list comprising naive stem cells, expanded pluripotent stem cells, reset stem cells, formative cells, induced pluripotent stem cells, primed pluripotent stem cells, 8 cell-like embryo-like cells (8CL), 4 cell-like embryo-like cells (4CL).

The term gastrulation refers to the process during embryonic development in which a blastula with a single layer of cells changes to a gastrula containing multiple layers of cells.

The term “pluripotent” or “pluripotency” refers to cells with the ability to give rise to progeny cells that can undergo differentiation, under the appropriate conditions, into cell types that collectively demonstrate characteristics associated with cell lineages from all of the three germinal layers (endoderm, mesoderm, and ectoderm). Pluripotent stem cells can contribute to all embryonic derived tissues of a prenatal, postnatal or adult animal. A standard art-accepted test, such as the ability to form a teratoma in 8-12 weeks old SCID mice, can be used to establish the pluripotency of a cell population, however identification of various pluripotent stem cell characteristics can also be used to detect pluripotent cells.

The term stem cells relates to cell that have the capacity to produce unaltered daughter cells (self-renewal; cell division produces at least one daughter cell that is identical to the parent cell) and to give rise to specialized cell types (potency). Stem cells include, but are not limited to, embryonic stem (ES) cells, embryonic germ (EG) cells, germline stem (GS) cells, human mesenchymal stem cells (hMSCs), adipose tissue-derived stem cells (ADSCs), multipotent adult progenitor cells (MAPCs), multipotent adult germline stem cells (maGSCs) and unrestricted somatic stem cell (USSCs), and haploid pluripotent embryonic stem cells. Generally, stem cells can divide without limit. After division, the stem cell may remain as a stem cell, become a precursor cell, or proceed to terminal differentiation. A precursor cell is a cell that can generate a fully differentiated functional cell of at least one given cell type. Generally, precursor cells can divide. After division, a precursor cell can remain a precursor cell, or may proceed to terminal differentiation.

The term “human embryonic stem cells” or “hESCs” means a human pluripotent cell or population of human pluripotent cells derived from an inner cell mass of a blastocyst. See Thomson et al., Science 282:1145-1147 (1998). In some embodiments, hESCs can be derived from the inner cell mass of blastocysts or morulae. In some embodiments, ES cells can be isolated from one or more blastomeres of an embryo, e.g., without destroying the remainder of the embryo. In some embodiments, ES cells can be produced by somatic cell nuclear transfer. In some embodiments, ES cells can be derived from fertilization of an egg cell with sperm or DNA, nuclear transfer, parthenogenesis or gynogenesis (duplicated maternal genome), androgenesis (duplicated paternal genome), or by means to generate ES cells, e.g., with homozygosity in the HLA region. In some embodiments, human ES cells can be produced or derived from a zygote, blastomeres, or blastocyst-staged mammalian embryo produced by the fusion of a sperm and egg cell, nuclear transfer, parthenogenesis, or the reprogramming of chromatin and subsequent incorporation of the reprogrammed chromatin into a plasma membrane to produce an embryonic cell. Exemplary human ES cells are known in the art and include, but are not limited to, MAO1, MAO9, ACT-4, No. 3, H1, H7, H9, H14 and ACT30 ES cells. In some embodiments, human ES cells, regardless of their source or the particular method used to produce them, can be identified based on, e.g., (i) the ability to differentiate into cells of all three germ layers, (ii) expression of at least Oct-4 and alkaline phosphatase, and/or (iii) ability to produce teratomas when transplanted into immunocompromised animals. In some embodiments, ES cells have been serially passaged as cell lines.

The term induced pluripotent stem cells (iPSC) refers to a type of pluripotent stem cell artificially derived from a non-pluripotent cell, such as an adult somatic cell (e.g., a fibroblast cell or other suitable somatic cell), by inducing expression of certain genes. iPSCs can be derived from any organism, such as a mammal. In some embodiments, iPSCs are produced from mice, rats, rabbits, guinea pigs, goats, pigs, cows, non-human primates or humans. iPSCs are similar to ES cells in many respects, such as the expression of certain stem cell genes and proteins, chromatin methylation patterns, doubling time, embryoid body formation, teratoma formation, viable chimera formation, potency and/or differentiability. Various suitable methods for producing iPSCs are known in the art. In some embodiments, iPSCs can be derived by transfection of certain stem cell-associated genes (such asOct-3/4 (Pouf51) and Sox2) into non-pluripotent cells, such as adult fibroblasts. Transfection can be achieved through viral vectors, such as retroviruses, lentiviruses, or adenoviruses. Additional suitable reprogramming methods include the use of vectors that do not integrate into the genome of the host cell, e.g., episomal vectors, or the delivery of reprogramming factors directly via encoding RNA or as proteins has also been described. For example, cells can be transfected with Oct3/4, Sox2, Klf4, and/or c-Myc using a retroviral system or with OCT4, SOX2, NANOG, and/or LIN28 using a lentiviral system. In some instances, c-Myc could be omitted as it is known to be associated with cancers. After 3-4 weeks, small numbers of transfected cells begin to become morphologically and biochemically similar to pluripotent stem cells, and can be isolated through morphological selection, doubling time, or through a reporter gene and antibiotic selection. In one example, iPSCs from adult human cells are generated by the method described by Yu et al., (2007) Induced pluripotent stem cell lines derived from human somatic cells. Science. 318 (5854) 1917-1920, González et al., (2011) Methods for making induced pluripotent stem cells: reprogramming a la carte. Nature Reviews Genetics. 12:231-242, Hochedlinger, K. & Jaenisch, R. (2006) Nuclear reprogramming and pluripotency. Nature. 441:1061-1067, and Takahashi K, et al. (2007) Induction of pluripotent stem cells from mouse embryonic and adult fibroblast cultures by defined factors. Cell. 131:861-72. In some embodiments, iPSCs are generated by a commercial source. In some embodiments, iPSCs are generated by a vendor. In some embodiments, iPSCs are generated by a contract research organization. Numerous suitable methods for reprogramming are known to those of skill in the art, and the present disclosure is not limited in this respect.

As the skilled person would be aware, a variety of methods are known for inducing differentiation of pluripotent stem cells into desired cell types. A non-limiting list of recent patent publications describing methods for inducing differentiation of stem cells into various cell fates follows: U.S. Patent Publication Nos.: 2007/0281355; 2007/0269412; 2007/0264709; 2007/0259423; 2007/0254359; 2007/0196919; 2007/0172946; 2007/0141703; 2007/0134215.

The term “primordial germ cell” as used herein refers to a diploid somatic cell capable of becoming a germ cell with the potential to differentiate into sperm or egg. The primordial germ cell may be a primordial germ cell like cell (PGCLC) differentiated from a pluripotent stem cell. The term human primordial germ cell-like cells or hPGCLCs may be a pluripotent stem cell-derived cell culture models of primordial germ cells which resembles the earlier stages of embryonic development. Germ cells, PGCs or PGCLCs can be induced to revert to a pluripotent stem cell-like cell, which is referred to embryonic germ cell (EGC).

In one embodiment, the in vitro human embryo organoid further comprises one or more of neuromesodermal progenitors, a heart tube, a beating heart, hindgut, neural tube, neural crest cells, neuronal progenitors, hematopoietic cells, endothelial cells, hepatocytes and cardiomyocytes. In some embodiments, the in vitro human embryo organoid generated according to the methods of the invention forms, for example, hematopoietic (stem/progenitor) cells, neural (stem/progenitor) cells (and optionally, more differentiated cells, such as subtype specific neurons, oligodendrocytes, etc), pancreatic cells {e.g., endocrine progenitor cell or pancreatic hormone-expressing cells), hepatocytes, cardiovascular (stem/progenitor) cells (e.g., cardiomyocytes, endothelial cells, smooth muscle cells), retinal cells, etc.

Stem cell-derived models aimed at modelling early embryonic development can be divided into integrated stem cell-derived models or non-integrated stem cell-derived models. Integrated stem cell-derived models contain all the integral parts of the whole conceptus, including its extraembryonic tissues. Integrated stem cell-derived models include the blastoid and the ETX (embryonic-trophoblast-extra-embryonic endoderm) embryoid assembled from trophectoderm stem cells, pluripotent stem cells, and extended potential stem cells. The integrated stem cell-based embryo model requires the assembly of blastocyst lineage stem cells into the blastoid and the blastoid-derived post-implantation embryoid. Conversely, non-integrated stem cell-derived models are generated to focus on a selected, specific developmental process, such as (but not limited to) gastrulation, development of the embryo axis and laterality or neural tube formation, and include gastruloids and amniotic sac models. Such models are restricted to the formation of specific body parts of the embryo. As such, in one embodiment, the in vitro human embryo organoid is non-integrated.

The term the three germ layers refers to the three primary layers formed in the earliest stages of embryonic development and comprise or consist of the endoderm (inner layer), the ectoderm (outer layer), and the mesoderm (middle layer).

The term “polynucleotide” (including, but not limited to “nucleotide sequence”, “nucleic acid”, “nucleic acid molecule”, “nucleic acid sequence”, and “oligonucleotide”) as used herein refers to a series of nucleotide bases (also called “nucleotides”) in DNA and RNA, and mean any chain of two or more nucleotides. In some embodiments, polynucleotides, nucleotide sequences, nucleic acids etc. can be chimeric mixtures or derivatives or modified versions thereof, single-stranded or double-stranded. In some such embodiments, modifications can occur at the base moiety, sugar moiety, or phosphate backbone, for example, to improve stability of the molecule, its hybridization parameters, etc. In general, a nucleotide sequence typically carries genetic information, including, but not limited to, the information used by cellular machinery to make proteins and enzymes. In some embodiments, a nucleotide sequence and/or genetic information comprises double- or single-stranded genomic DNA, RNA, any synthetic and genetically manipulated polynucleotide, and/or sense and/or antisense polynucleotides. In some embodiments, nucleic acids containing modified bases.

The terms “protein,” “peptide” and “polypeptide” as used herein are used interchangeably to refer to a sequential chain of amino acids linked together via peptide bonds. The terms include individual proteins, groups or complexes of proteins that associate together, as well as fragments or portions, variants, derivatives and analogs of such proteins. Unless otherwise specified, peptide sequences are presented herein using conventional notation, beginning with the amino or N-terminus on the left, and proceeding to the carboxyl or C-terminus on the right. Standard one-letter or three-letter abbreviations can be used.

The level of gene expression, including the level of gene upregulation or downregulation, can be measured by known procedures of which the skilled person would be well aware. In general, gene upregulation comprises any detectable increase in the production of a gene product and downregulation comprises any detectable decrease in the production of a gene product.

There may be differential gene expression in different regions of the in vitro human embryo organoid. For example, in one embodiment, in vitro human embryo organoid is polarised along the rostro-caudal axis into an anterior region and a posterior region. In one embodiment, the anterior region and posterior regions comprise differential expression of one or more one or more markers indicative of primordial germ cell-like cells.

In one embodiment, GATA6 gene is expressed in the anterior region and TBXT gene or CDX2 gene is expressed at the posterior region. In some embodiment, the anterior region comprises upregulation or downregulation of one of more genes selected from a list comprising GATA6, MYH10, TNNI1, TNNI2, and HAND1 and/or the posterior region comprises upregulation or downregulation of one of more genes selected from a list comprising CDX2 and CDX1. In some embodiment, the posterior region comprises markers for neuromesodermal progenitors, wherein the markers for neuromesodermal progenitors are one or more genes selected from a list comprising TBXT, SOX2, NKX1.2, HOXB8, and HOXC9.

In one embodiment, human embryo organoid comprises neural precursors, wherein the neural precursors are one or more genes selected from a list comprising SOX2, ZIC5, POU3F2, neural progenitors, wherein the neural progenitors are one or more genes selected from a list comprising ISI1, ZEB2, FOXC1, neural crest markers, wherein the neural crest are one or more genes selected from a list comprising FOXD3, S100B, HOXB9, SOX2, SOX10, CRABP1, TFAP2A, TFAP2B, neuron markers, wherein the neuron markers are one or more genes selected from a list comprising STMN2, ASCL1, ELAVL3, NEUROG1, and NEUROD4, neural tube markers, wherein the neural tube markers are one or more genes selected from a list comprising SOX3, PAX6, PAX7, and HES5, spinal cord markers, wherein the spinal cord markers are one or more genes selected from a list comprising OLIG3, SOX1, ZIC1, PAX7, and FGF8, and neural ectoderm markers, wherein the neural ectoderm markers are one or more genes selected from a list comprising SOX1 and PAX6.

In one embodiment, the in vitro human embryo organoid comprises Hematopoietic Stem and Progenitor Cells (HSPC), wherein the HSPCs comprise one or more genes selected from a list comprising RUNX1, SOX7 and CD34.

In one embodiment, the in vitro human embryo organoid comprises one or more selected from a list comprising hematopoietic cells CD45⁺RUNX1⁺, RUNX1⁺CD45⁻, and CD45⁺RUNX1⁻, CD31⁺CD45⁻, CD31⁺CD45⁺, CD31⁻CD45⁺, CD31⁺CD34⁺CD45⁺, CD34⁺CD45⁻, CD34⁻CD45⁺.

In one embodiment, the hematopoietic cells further comprise one or more genes selected from a list comprising CD45, MYB, ITGA2B, SPN, GFI1B, SPINK2 and CD44.

In one embodiment, the hematopoietic cells further comprise markers for megakaryocytes, wherein the markers for megakaryocytes comprise one or more genes selected from a list comprising GP1BA, GP1BB and NFE2.

In one embodiment, the hematopoietic cells further comprise markers for macrophages, wherein the markers for macrophages comprise one or more genes selected from a list comprising CD68, CD86, MRC1 and TREM2.

In one embodiment, the hematopoietic cells further comprise markers for eosinophil, basophil and mast cell progenitors, wherein the eosinophil, basophil and mast cell progenitors comprise one or more genes selected from a list comprising GATA2, IL1B, MYC, STAT5A and SOX4.

In one embodiment, the hematopoietic cells further comprise one or more genes selected from a list comprising lymphocytes CD52, CD69, HOPX, SPINK2, IL7R and NKG7.

In one embodiment, the in vitro human embryo organoid expresses one or more markers indicative of primordial germ cell-like cells selected from a group comprising NANOS3, POU5F1, PDPN, TFAP2C, PRDM1, PRDM14, CD38 and NANOG.

In one embodiment, the in vitro human embryo organoid expresses one or more markers indicative of neuronal precursor cells selected from a group comprising S100B, FOXD3, SOX10, ELAVL3, NEUROD4, NEUROG1, SOX2, SOX10, CRABP1, TFAP2A, TFAP2B, STMN2, ASCL1, ELAVL3, NEUROG1, NEUROD4, SOX3, PAX6, PAX7, HES5, OLIG3, SOX1, ZIC1, PAX7 and FGF8.

In one embodiment, the anterior region expresses TNNT2 and MYH2 genes.

In one embodiment, the in vitro human embryo organoid comprises contractile cardiomyocytes, wherein the markers for cardiomyocytes are one or more genes selected from a list comprising NKX2-5 and MYH2, TNNI1, ACTC1, TNNT2, MYH6, MYL7 and IRX3.

In one embodiment, the in vitro human embryo organoid comprises amnion cells, wherein the markers for amnion cells are one or more genes selected from a list comprising GABRP, VTCN1, WNT6 and TFAP2A,

In one embodiment, the in vitro human embryo organoid comprises mesenchyme cells, wherein the markers for mesenchyme cells are one or more genes selected from a list comprising PCOLCE, COL1A2, COL6A3 and HAND1.

In one embodiment, the in vitro human embryo organoid comprises hepatocytes, wherein the markers for hepatocytes are one or more genes selected from a list comprising AFP, ALB and CEBPA.

In one embodiment, the in vitro human embryo organoid comprises blood progenitors, wherein the markers for blood progenitors are one or more genes selected from a list comprising PECAM1/CD31, CD34, CDH5, RUNX1, PTPRC/CD45 and CD44.

In one embodiment, the in vitro human embryo organoid comprises splanchnic mesoderm, wherein the markers for splanchnic mesoderm are one or more genes selected from a list comprising WT1, LHX2 and GATA4.

In one embodiment, the in vitro human embryo organoid comprises paraxial mesoderm, wherein the markers for paraxial mesoderm are one or more genes selected from a list comprising MEOX1, SIX4 and FOXC2.

In one embodiment, the in vitro human embryo organoid comprises erythroid cells, wherein the markers for erythroid cells are one or more genes selected from a list comprising GATA1, KLF1, HBA1, HBA2, HBE1, HBE2, HBG1 and HBG2.

In one embodiment, the in vitro human embryo organoid comprises endothelial cells, wherein the markers for endothelial cells are one or more genes selected from a list comprising CD34, CD31, CDH5, KDR, SOX7, THY1, ITGB1, CXCR4, NRARP, GJA4, IGFBP4, VEGF1, FLT1, TMEM88, TPM1, PRTG, HOXB7, MEIS2, RNF19A, CAV1, REN, BNIP3, STC1 and FGF23.

In one embodiment, the in vitro human embryo organoid comprises hepatocytes (foetal liver progenitors), wherein the markers for hepatocytes are one or more genes selected from a list comprising AFP, APOA1, APOB, ALB and TF.

In one embodiment, the hepatocyte may be derived from a foetal liver progenitor.

In one embodiment, the in vitro human embryo organoid comprises musculoskeletal precursors, wherein the musculoskeletal precursors are one or more genes selected from a list comprising NEFM, SIX1 and TNNT1.

In one embodiment, the in vitro human embryo organoid comprises hindgut, wherein the markers for hindgut are one or more genes selected from a list comprising CDX2, SOX2, SOX17, SHH and EPCAM.

As stated above, in one embodiment, the in vitro human embryo organoid is derived in vitro from one or more human embryonic stem cells (ESCs) and in another embodiment, the in vitro human embryo organoid is derived in vitro from one or more human induced pluripotent stem cells (iPSCs). The starting cell type does not alter the methods of the invention described herein.

The invention also comprises a method of producing an in vitro human embryo organoid at a post-gastrulation stage of development comprising the steps of:
a) treating human embryonic stem cells or human induced pluripotent stem cells to disassociate them into single cells,
b) formation of aggregates,
c) advancement of aggregates to human embryo organoid at the post-gastrulation stage of development; and
d) advancement of aggregates to human embryo organoid at the post-gastrulation stage of development, wherein the aggregates are incubated in the presence of a GSK3β inhibitor, preferably wherein the GSK3β inhibitor is Chiron, and a TGF-β inhibitor, preferably wherein the TGF-β inhibitor is SB43.

In one embodiment, the glycogen synthase kinase-3 (GSK-3)-β inhibitor may be selected from a list comprising CHIRON99021, 6-BIO, SB415286, and SB216763. In a further embodiment, WNT3 may be used in place of a GSK-3-β inhibitor. Therefore, step d) of the abovementioned method may comprise advancement of aggregates to human embryo organoid at the post-gastrulation stage of development, wherein the aggregates are incubated in the presence of WNT3 and a TGF-β inhibitor.

In one embodiment, the Transforming growth factor (TGF)-β inhibitor may be selected from a list comprising SB431542, SB505124, SB525334, A83-01, and Galunisertib.

In one embodiment, step a) is performed in the presence of a TGF-β, preferably wherein the TGF-β is Activin-A, and a GSK3β inhibitor, preferably wherein the GSK3β inhibitor is Chiron. In one embodiment the TGF-β is Nodal.

In one embodiment, step b) is performed in the presence of a GSK3β inhibitor, preferably wherein the GSK3β inhibitor is Chiron.

In one embodiment, step c) is performed in the presence of a GSK3β inhibitor, preferably wherein the GSK3β inhibitor is Chiron, and a TGF-β inhibitor, preferably wherein the TGF-β inhibitor is SB43.

In one embodiment, step d) further comprises incubating the in vitro human embryo organoid in a rotary culture system.

In one embodiment, step b) and/or step d) is performed in the presence of a Rho kinase inhibitor (ROCKi). In another embodiment, the Rho kinase inhibitor may be ki-23095 or VAS-092. In a further embodiment, step b) and/or step d) is performed in the presence of a Rho kinase inhibitor (ROCKi) for the first 24 hours. ROCKi may therefore be present during the first 24 hours of culturing at the point when the cells are single cells prior to aggregation and at the step of adding the factors necessary to advance aggregates to human embryo organoid.

In one embodiment, one or more of the method steps are performed under conditions of about 1% to about 10% oxygen and 1% to about 6% carbon dioxide, preferably about 5% oxygen and about 5% carbon dioxide, optionally, wherein all of the method steps are performed under conditions of about 1% to about 10% oxygen and 1% to about 6% carbon dioxide, preferably about 5% oxygen and about 5% carbon dioxide. Conditions of 5% oxygen and 5% carbon dioxide are hypoxic conditions. In a further embodiment, all of the method steps are performed under conditions of about 1% to about 10% oxygen and 1% to about 6% carbon dioxide In a yet further embodiment, one or more of the method steps may be performed under conditions of any one of about 1% oxygen, or 2% oxygen, or 3% oxygen, or 4% oxygen, or 5% oxygen, or 6% oxygen, or 7% oxygen, or 8% oxygen, or 9% oxygen, or 10% oxygen and may be used in combination with any one of 1% carbon dioxide, or 2% carbon dioxide, or 3% carbon dioxide, or 4% carbon dioxide, or 5% carbon dioxide, or 6% carbon dioxide.

In a further method according to the invention, there is provided a method of advancing an in vitro human embryo organoid at the peri-gastrulation stage of development to a post-gastrulation stage of development comprising incubating the human embryo organoid at the peri-gastrulation stage of development in the presence of a GSK3β inhibitor, preferably wherein the GSK3β inhibitor is Chiron, and a TGF-β inhibitor, preferably wherein the TGF-β inhibitor is SB43. One embodiment of this method, further comprising incubating the in vitro human embryo organoid in a rotary culture system.

In one embodiment, one or more of the method steps are performed under conditions of about 1% to about 10% oxygen and 1% to about 6% carbon dioxide, preferably about 5% oxygen and about 5% carbon dioxide, optionally, wherein all of the method steps are performed under conditions of about 1% to about 10% oxygen and 1% to about 6% carbon dioxide, preferably about 5% oxygen and about 5% carbon dioxide. Conditions of 5% oxygen and 5% carbon dioxide are hypoxic conditions. In a further embodiment, all of the method steps are performed under conditions of about 1% to about 10% oxygen and 1% to about 6% carbon dioxide In a yet further embodiment, one or more of the method steps may be performed under conditions of any one of about 1% oxygen, or 2% oxygen, or 3% oxygen, or 4% oxygen, or 5% oxygen, or 6% oxygen, or 7 oxygen, or 8% oxygen, or 9% oxygen, or 10% oxygen and may be used in combination with any one of 1% carbon dioxide, or 2% carbon dioxide, or 3% carbon dioxide, or 4% carbon dioxide, or 5% carbon dioxide, or 6% carbon dioxide.

As previously discussed, the in vitro human embryo organoid comprises derived by the methods of the invention may comprise cells expressing markers characteristic of endodermal cells, cells expressing markers characteristic of mesodermal cells, cells expressing markers characteristic of ectodermal cells; and primordial germ cell-like cells (hPGCLCs). In a further embodiment, the in vitro human embryo organoid further comprises one or more of neuromesodermal progenitors, a heart tube, a beating heart, hindgut, neural tube, neural crest cells, neuronal progenitors, hematopoietic cells, endothelial cells, hepatocytes and cardiomyocytes.

In one embodiment of the methods described herein, in vitro human embryo organoid is cultured for any one of at least 3, 4, 5, 6, 7, 8, 9, 10, 11, 12, 13, or 14 day.

In one embodiment, the invention may comprise a progenitor cell or derivative thereof obtainable by the method disclosed herein. In one embodiment, the progenitor cell or derivative thereof is an endodermal cell, mesodermal cell, ectodermal cell, neural crest cell, neuronal progenitor, neuronal precursor cell, hematopoietic cell, endothelial cell, hepatocyte cardiomyocyte, HSPC, megakaryocyte, macrophage, eosinophil, basophil, mast cell, lymphocyte, primordial germ cell-like cell, amnion cell, mesenchyme cell, hepatocytes, foetal liver progenitor, blood progenitor, erythrocyte, myeloid cell, endothelial cell, musculoskeletal precursor cell, ESC and/or iPSC.

In one embodiment, the invention may comprise a kit comprising an in vitro human embryo organoid at a post-gastrulation stage of development according to the invention.

In one embodiment, the invention may comprise a system for drug screening for teratogenic and therapeutic purposes comprising an in vitro human embryo organoid at a post-gastrulation stage of development according to the invention. A teratogen is an agent that causes an abnormality following fetal exposure during pregnancy and the in vitro human embryo organoid at a post-gastrulation stage of development could be used to screen teratogens rather than using human embryos, which are difficult to obtain and have moral and ethical issues related to their use.

Unless otherwise defined herein, scientific and technical terms used in connection with the present disclosure shall have the meanings that are commonly understood by those of ordinary skill in the art.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

All documents mentioned in this specification are incorporated herein by reference in their entirety, including references to gene accession numbers.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein. Unless context dictates otherwise, the descriptions and definitions of the features set out above are not limited to any particular aspect or embodiment of the invention and apply equally to all aspects and embodiments which are described.

### EXAMPLES

The invention is further described in the non-limiting examples.

### Example 1

### Embryo Organoids for Early Human Development

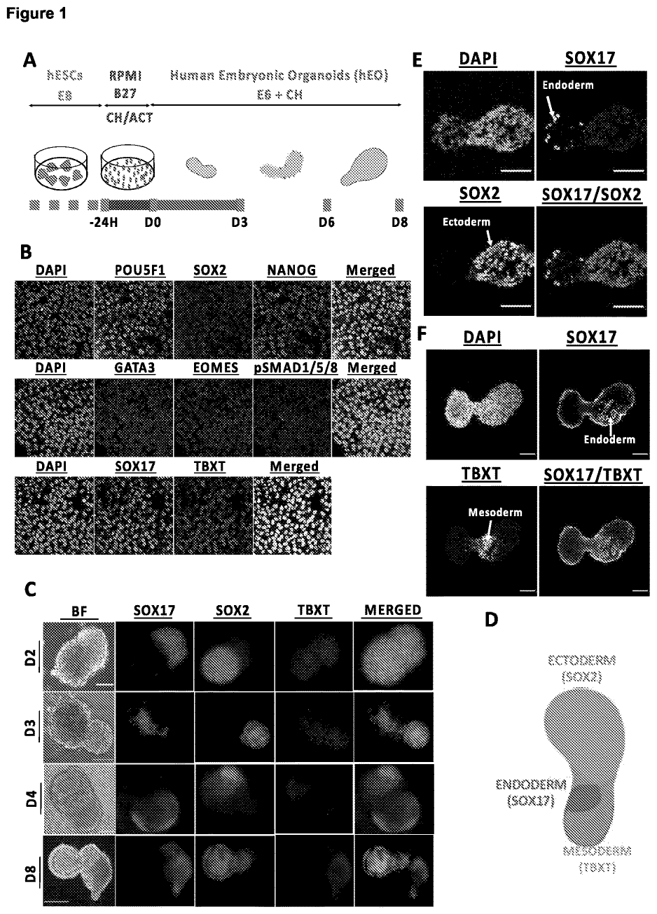

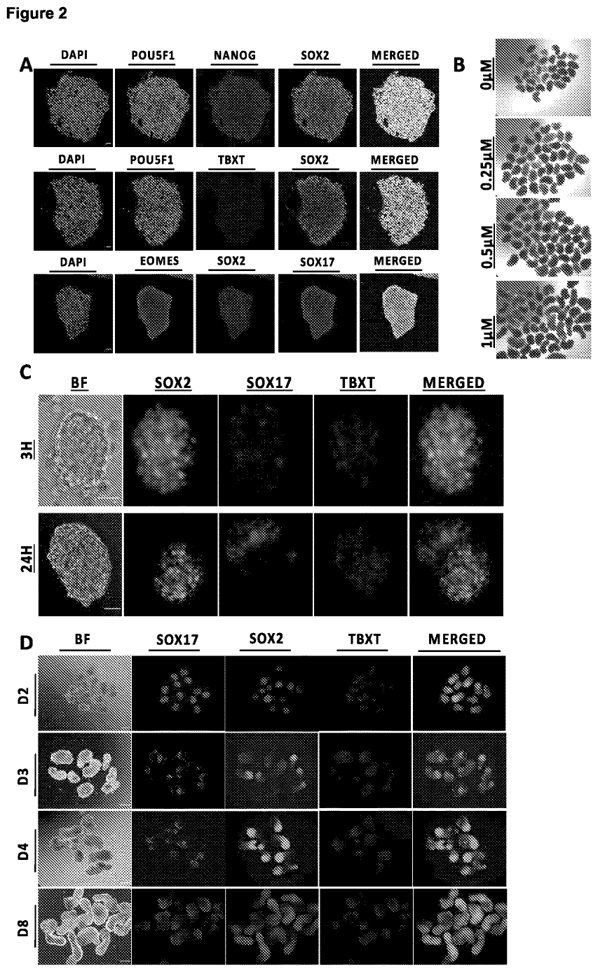

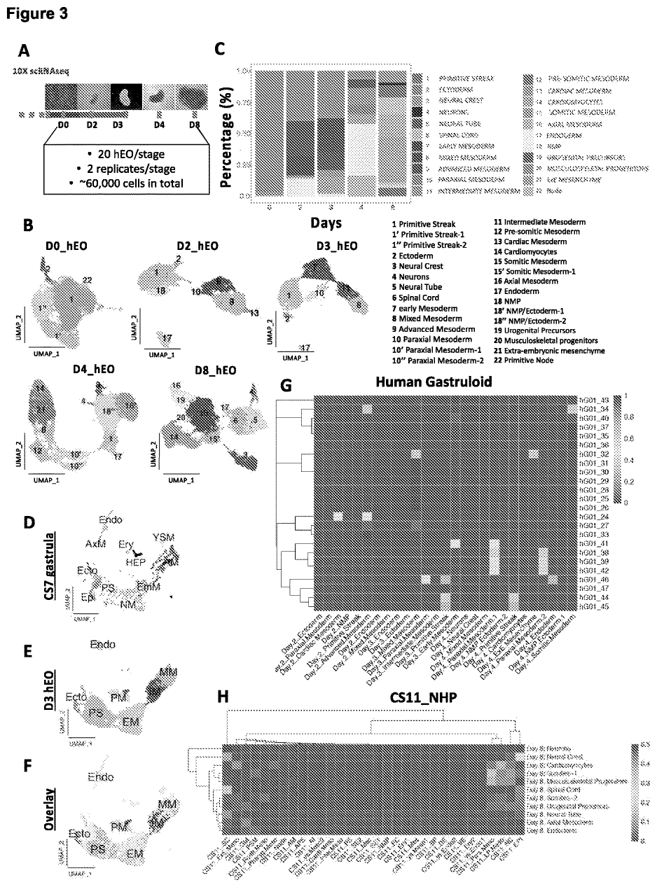

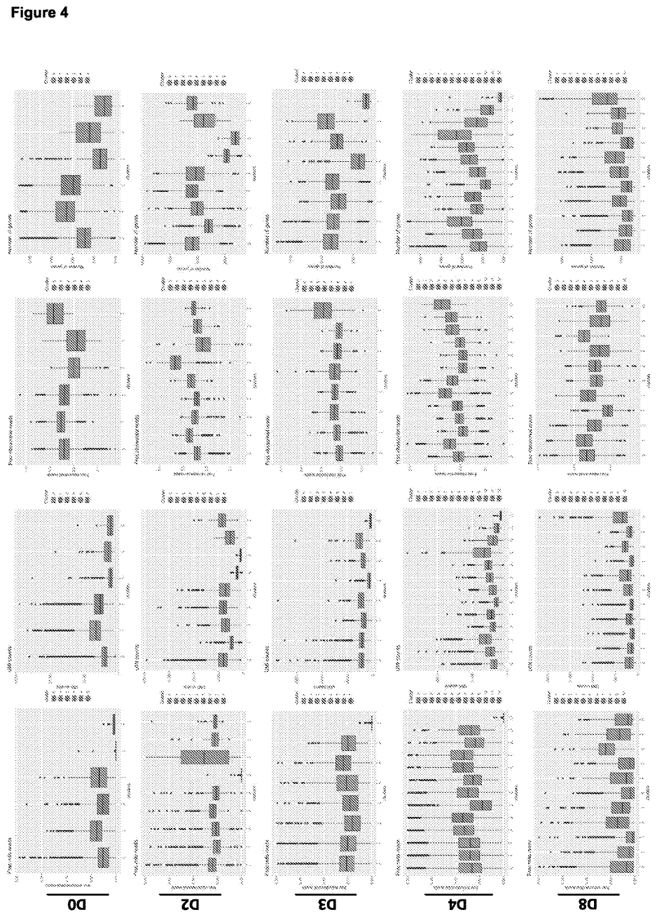

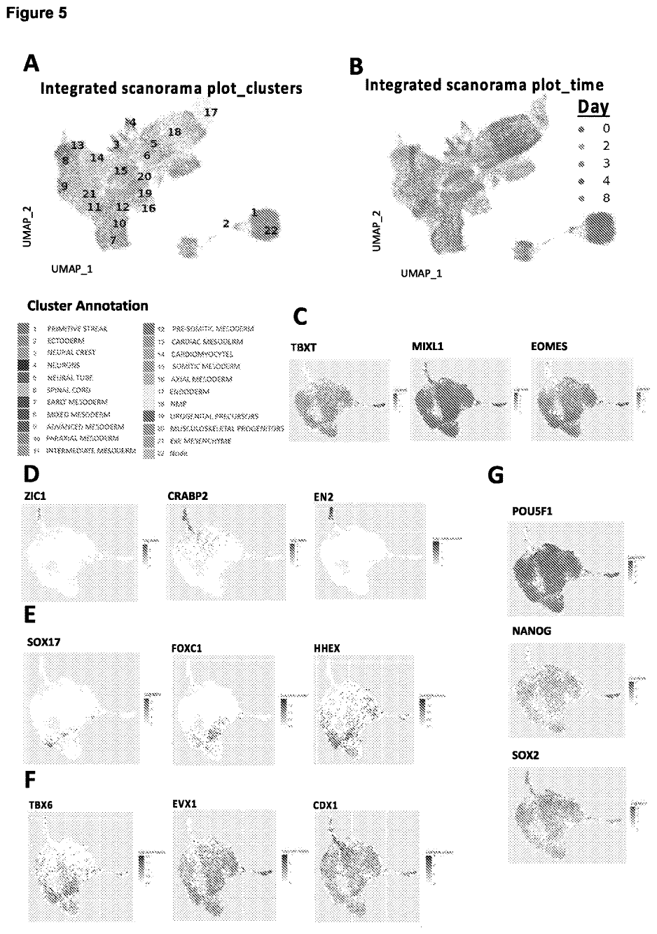

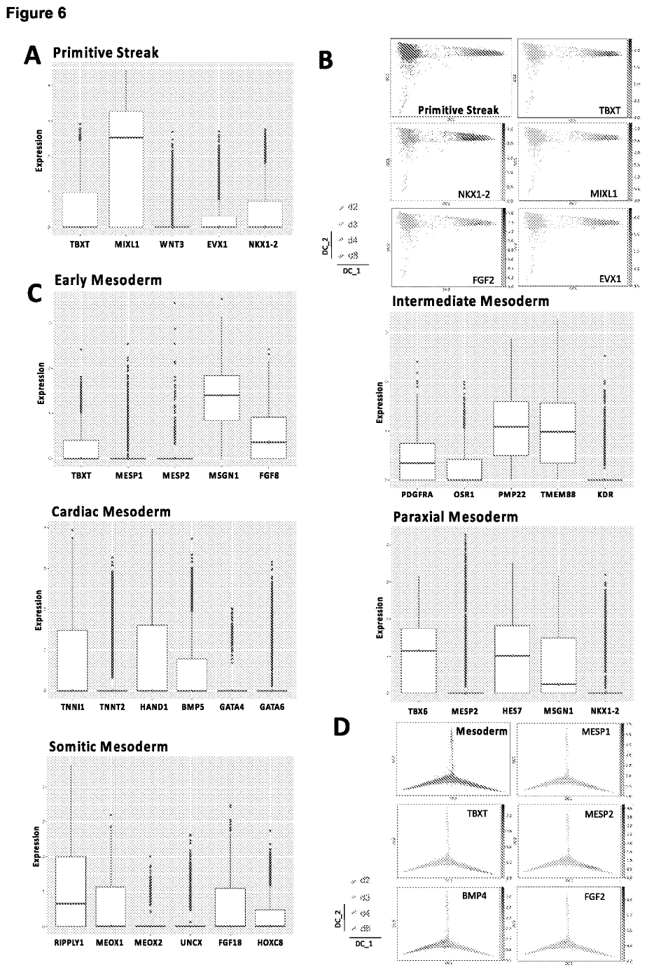

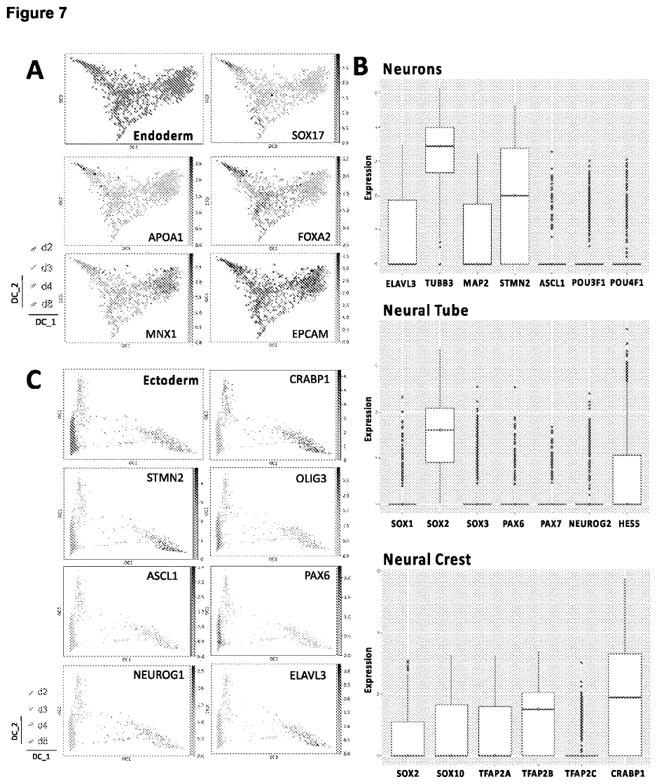

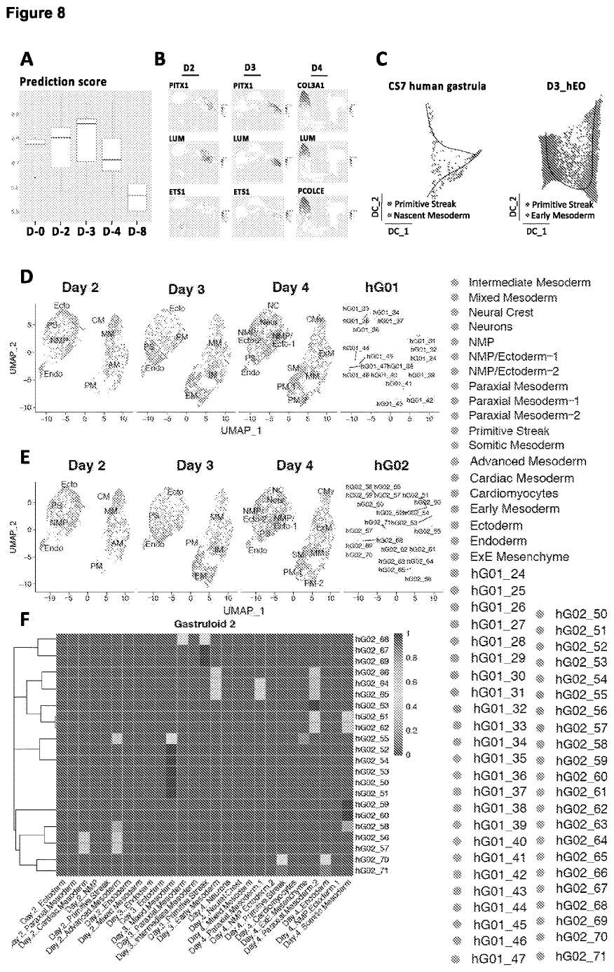

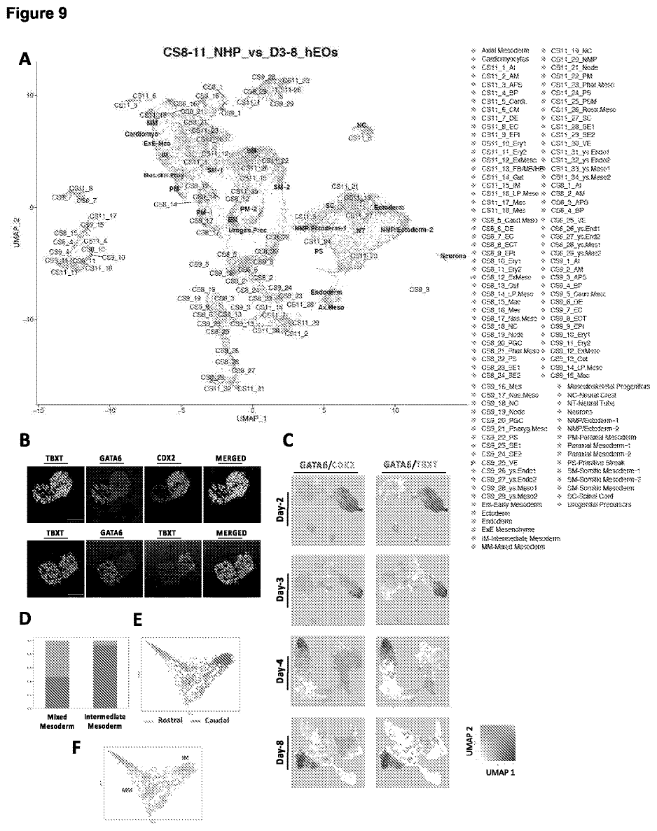

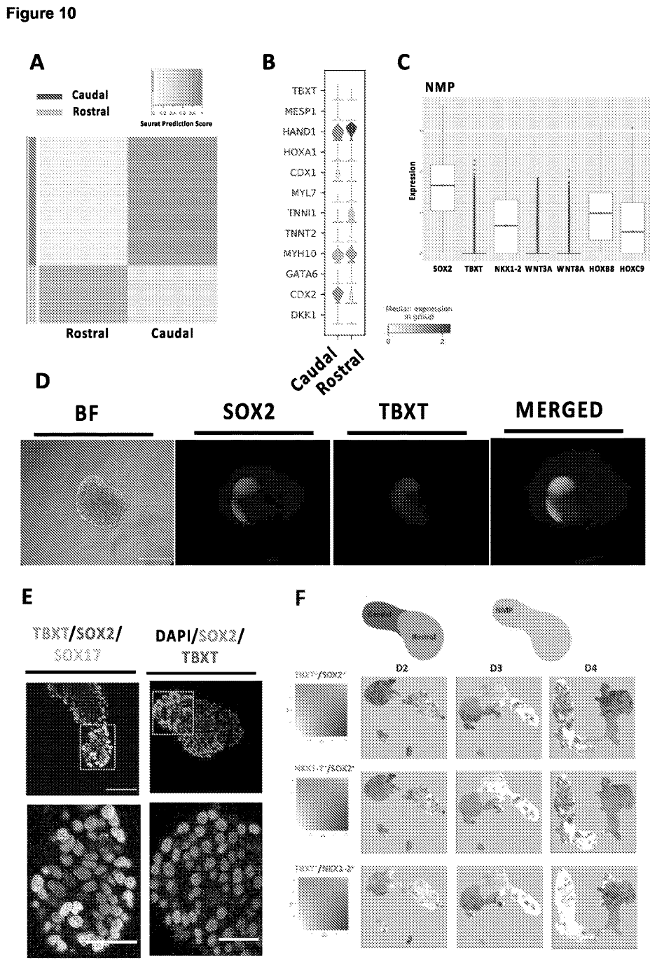

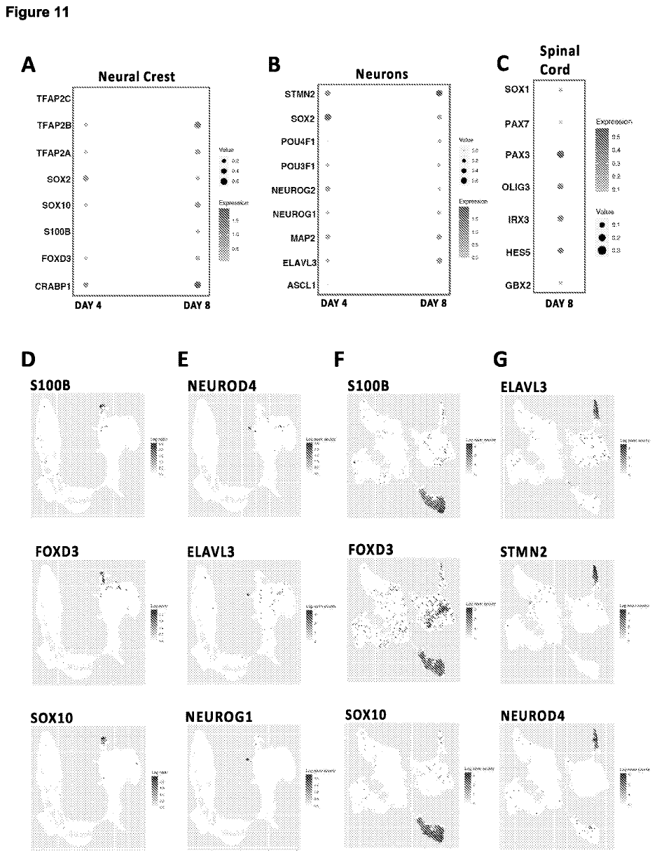

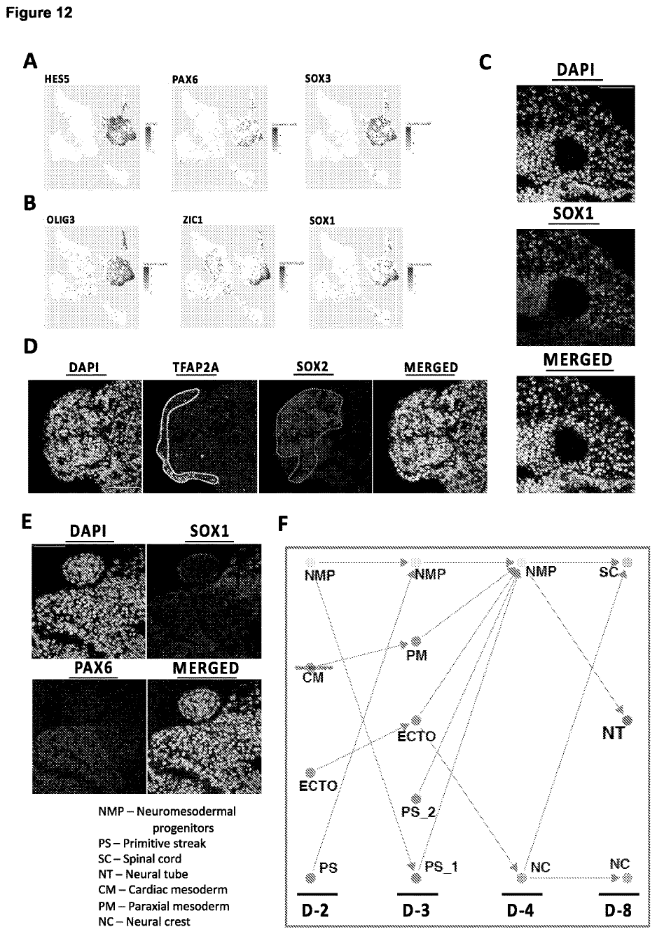

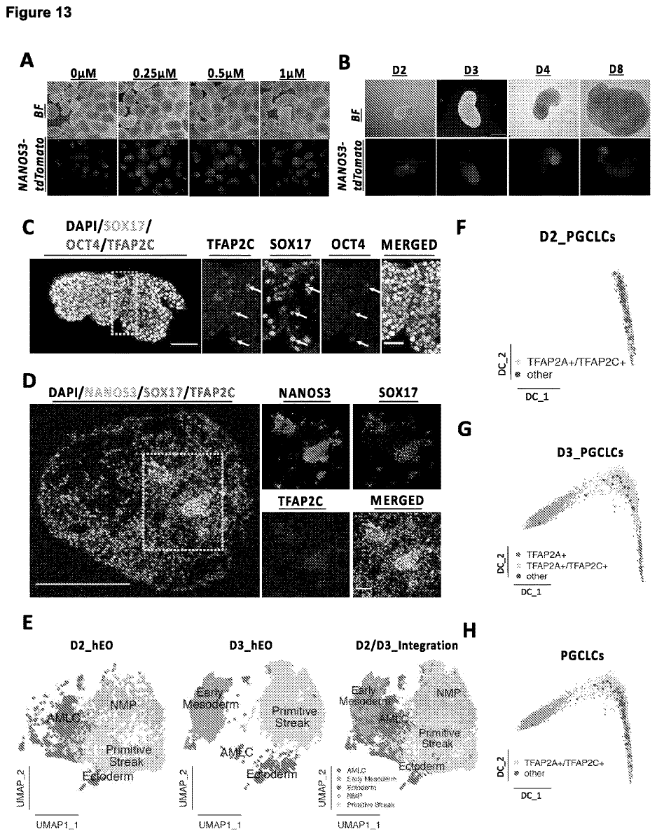

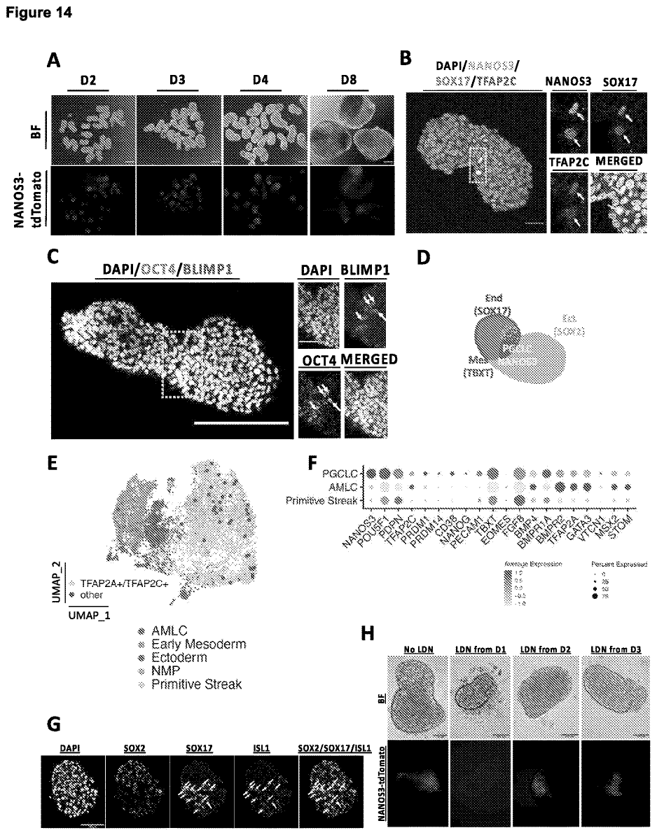

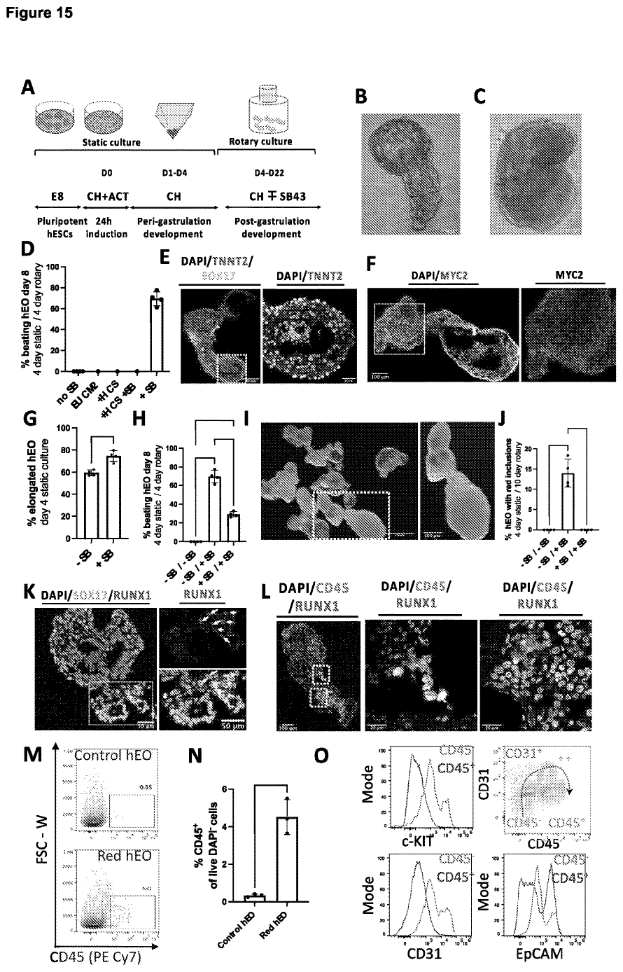

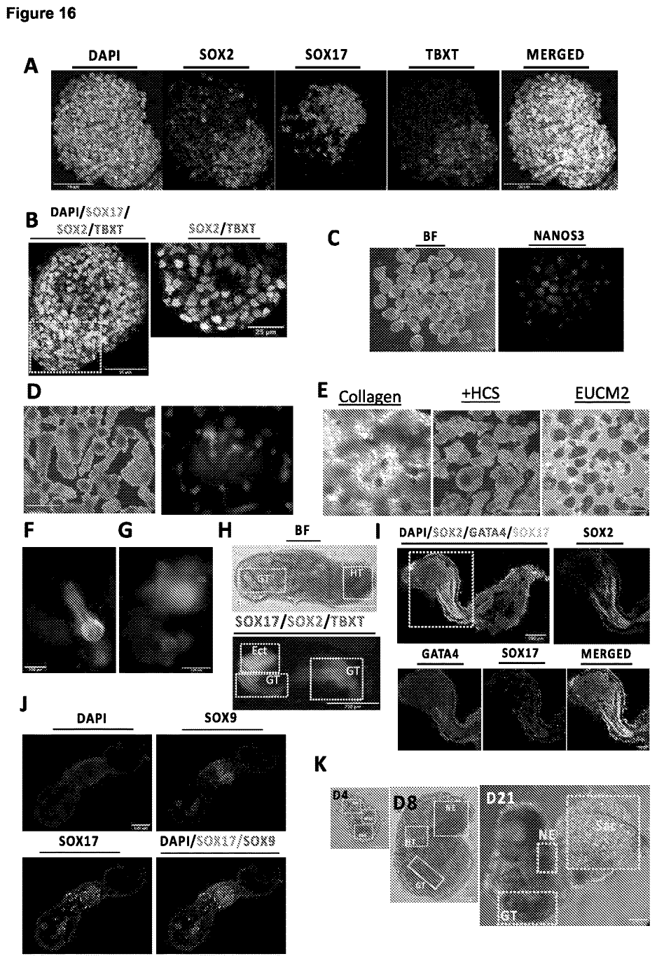

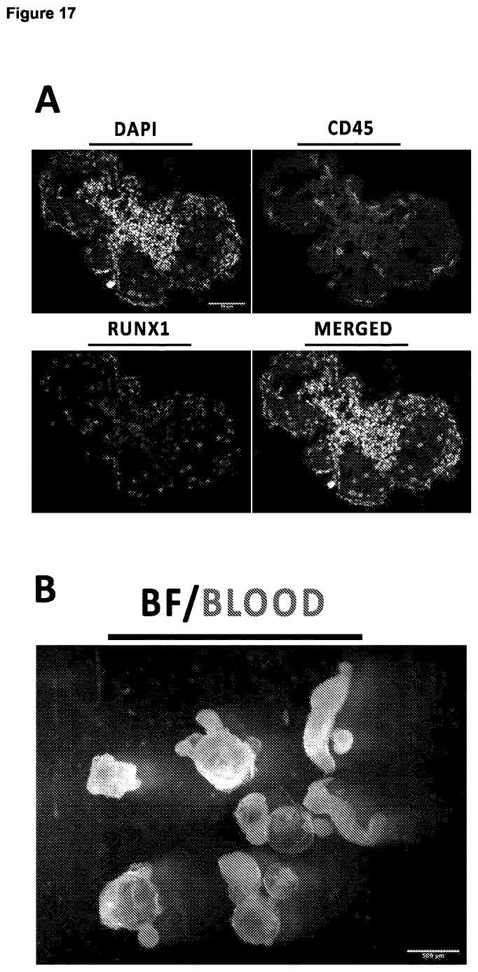

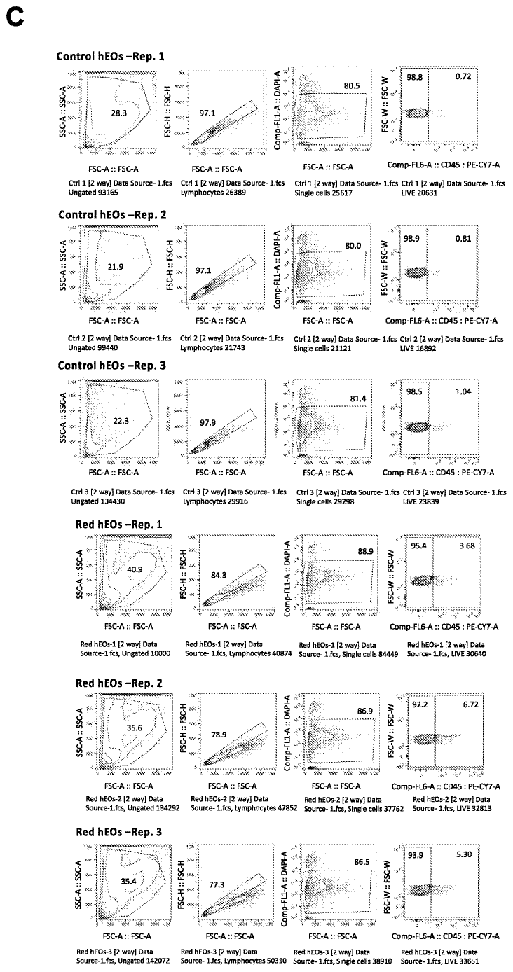

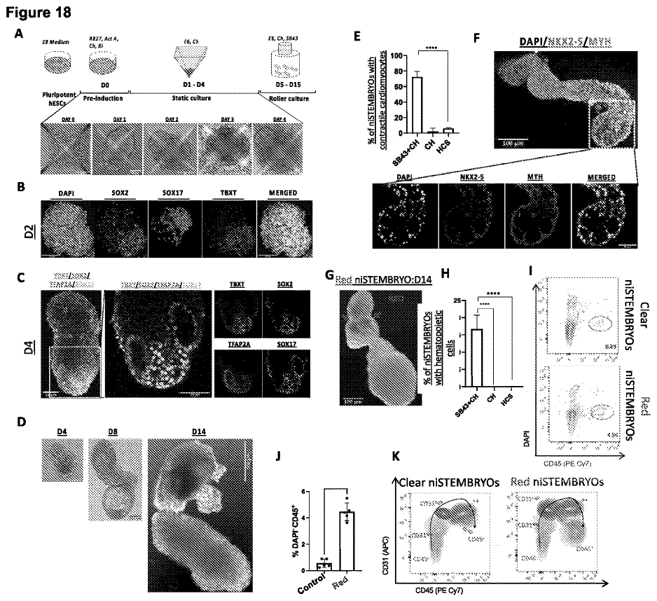

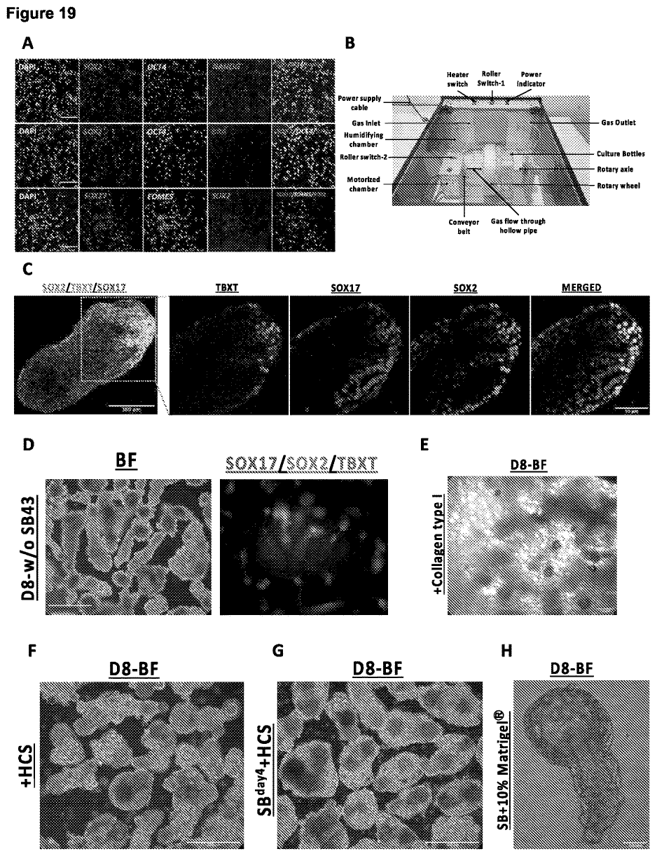

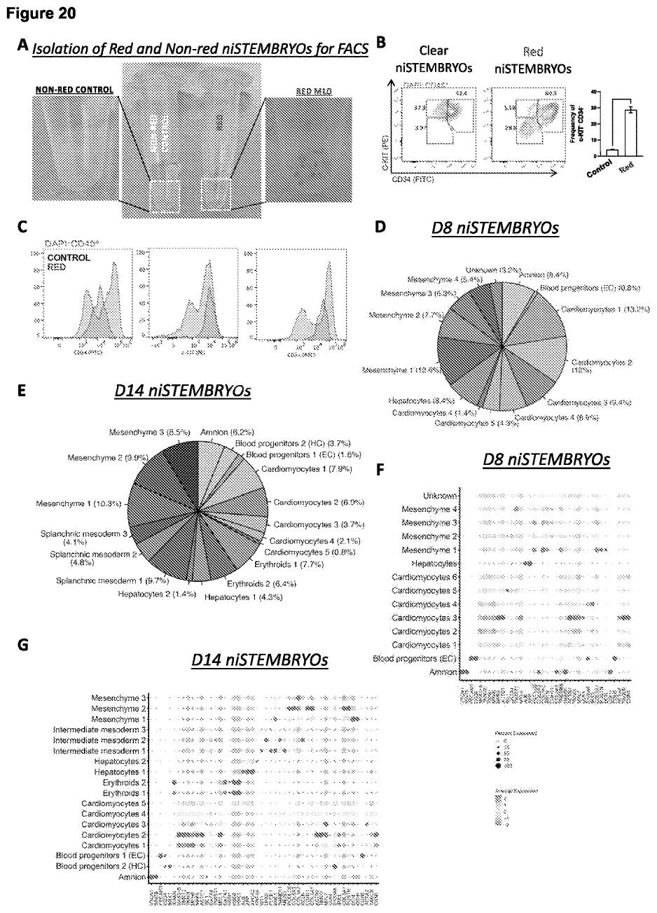

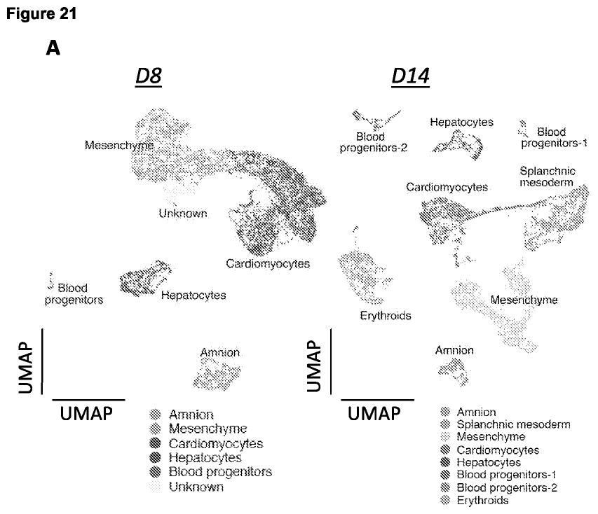

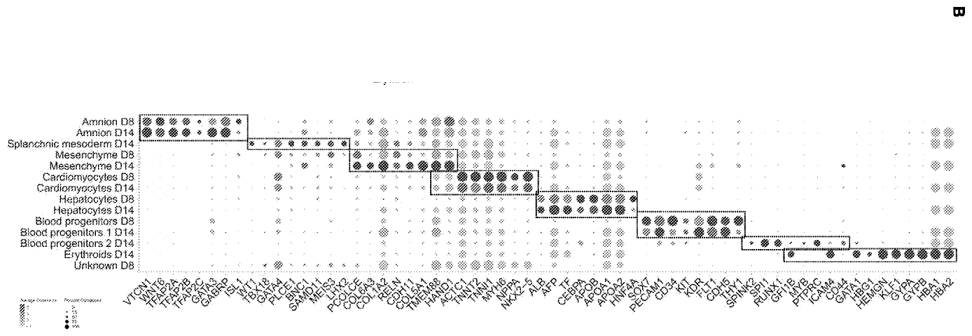

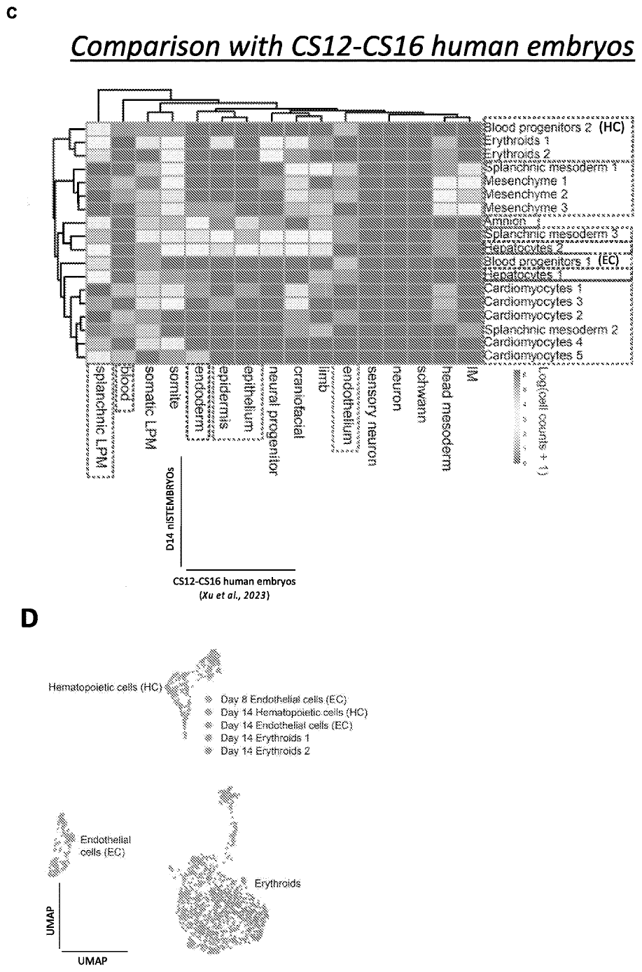

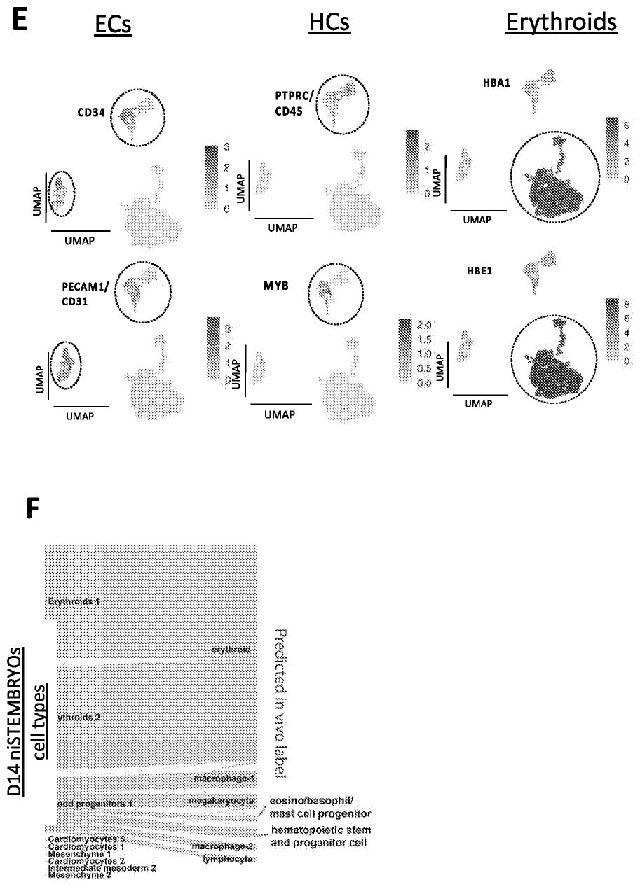

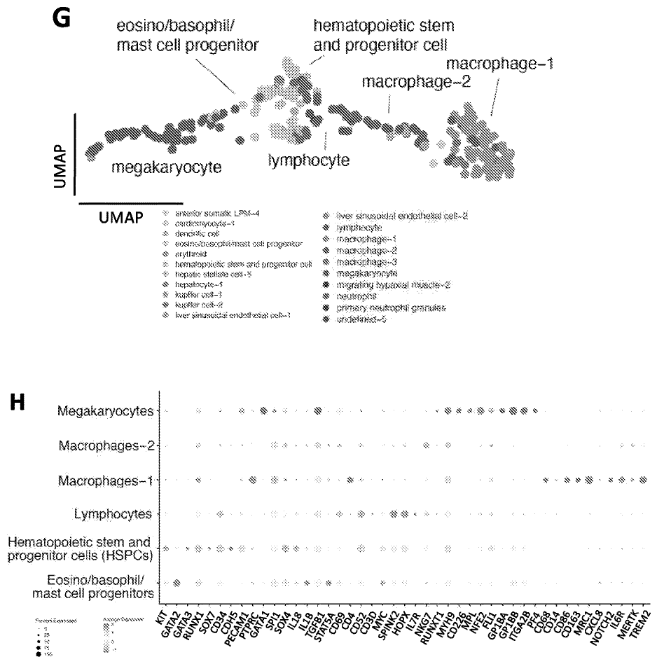

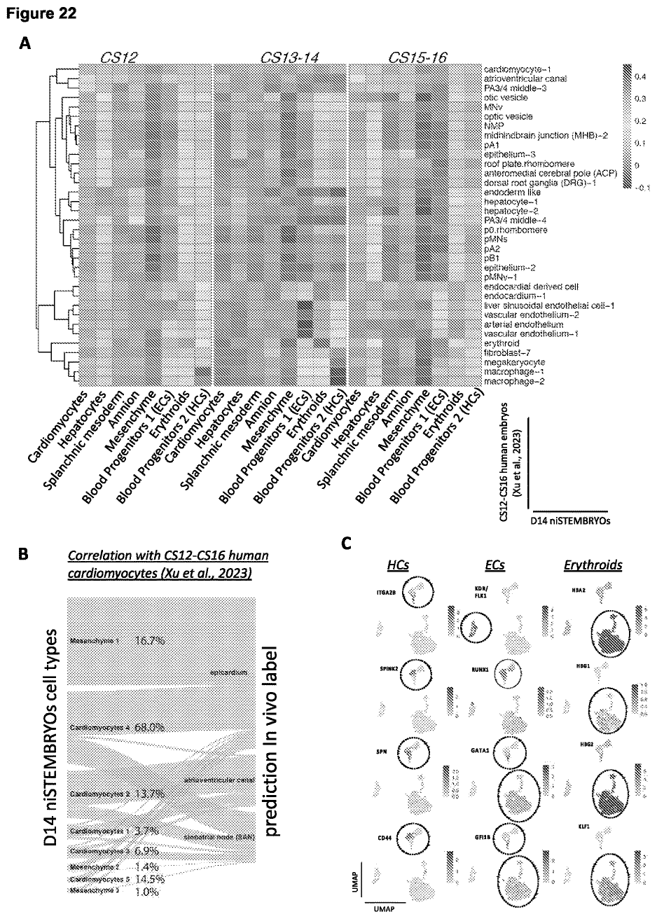

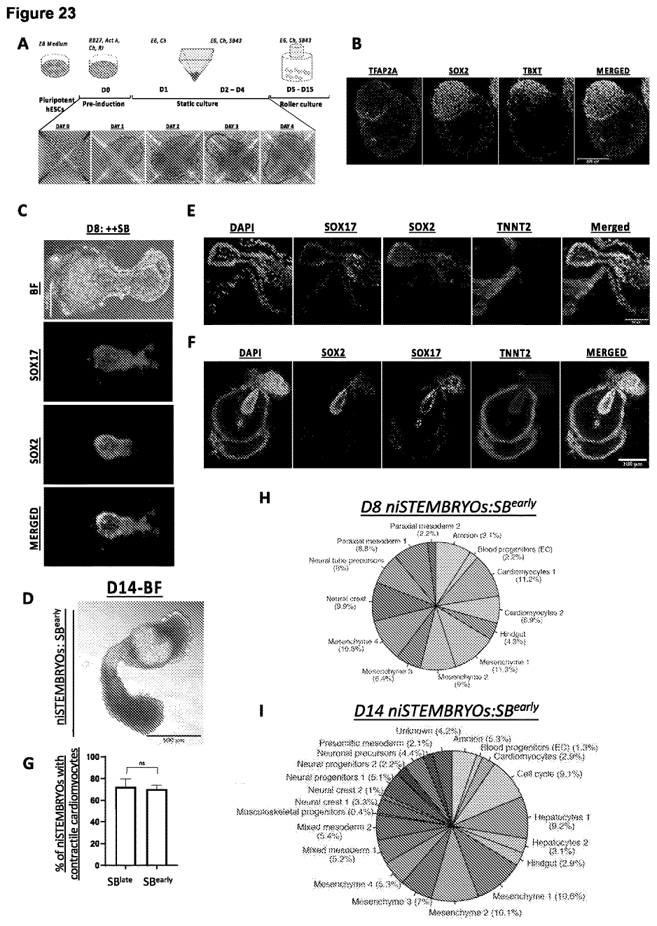

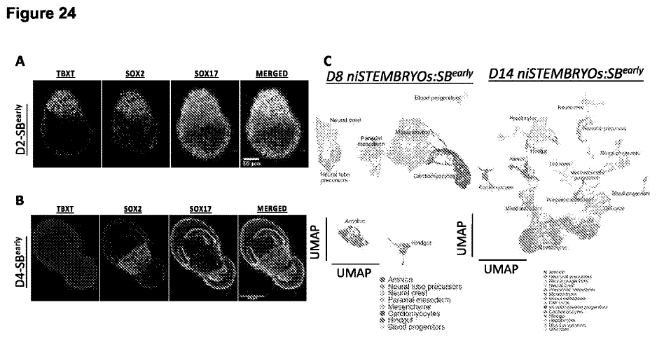

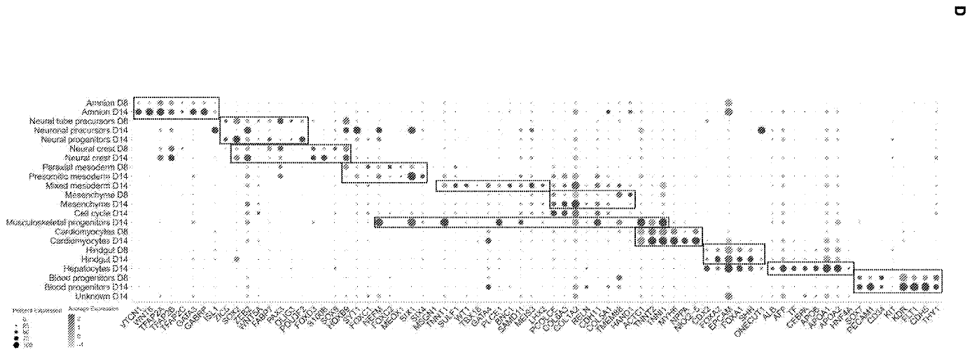

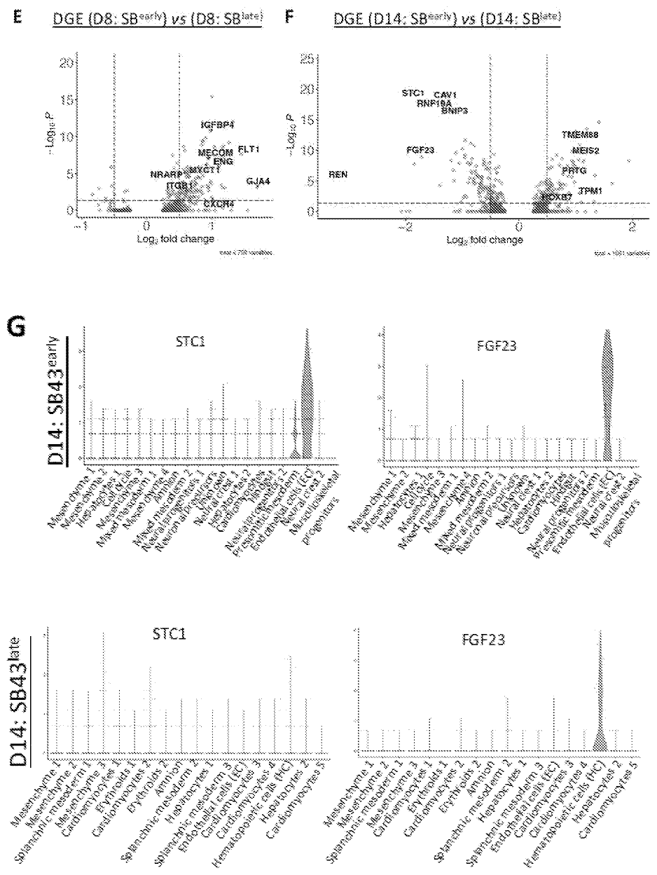

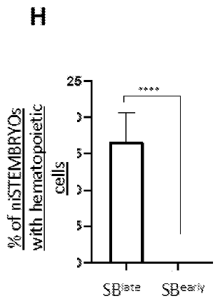

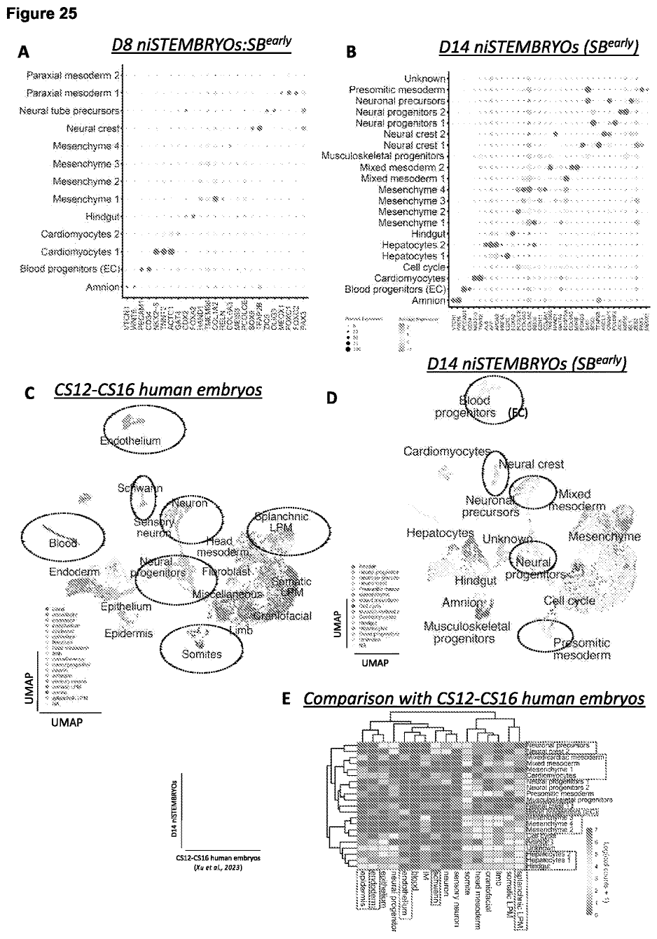

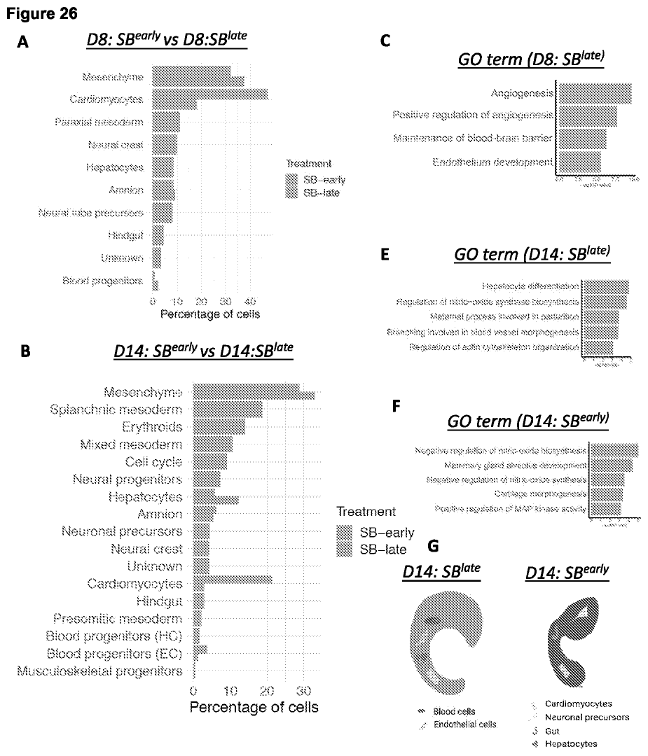

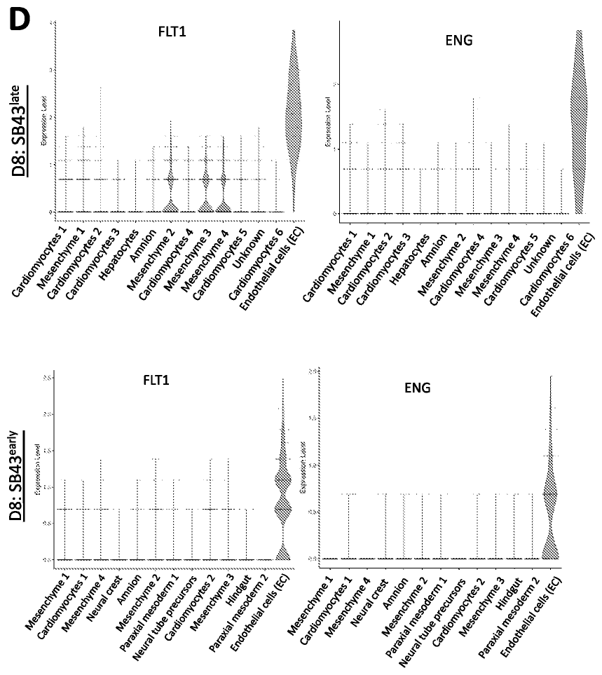

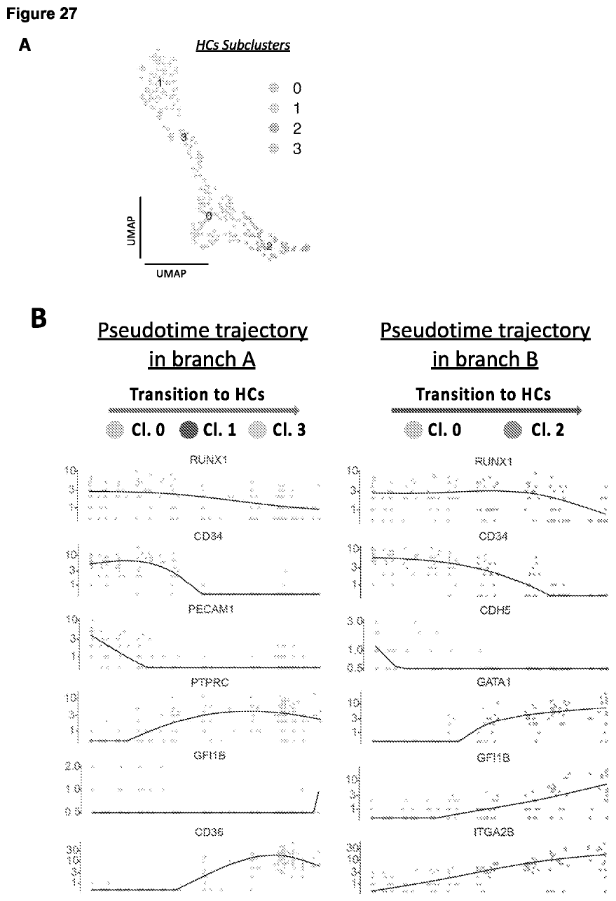

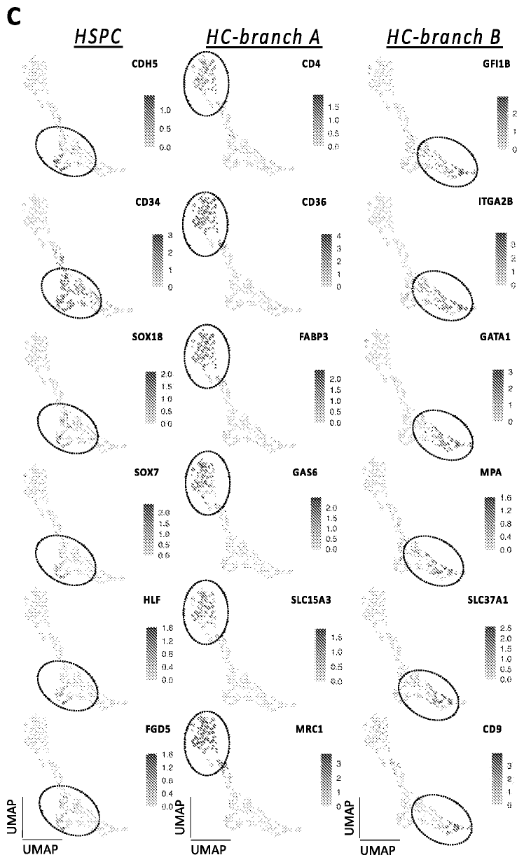

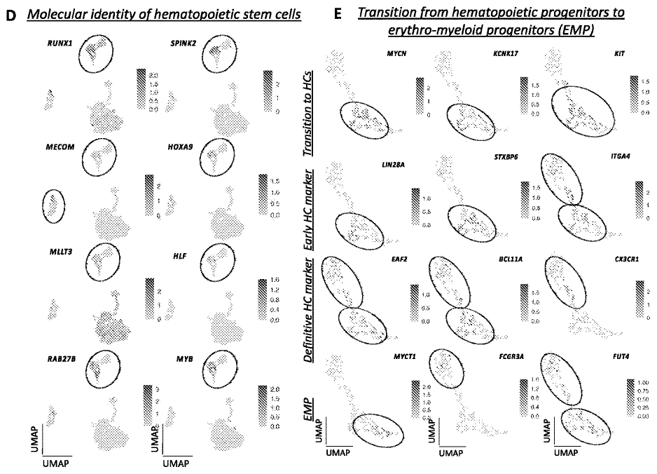

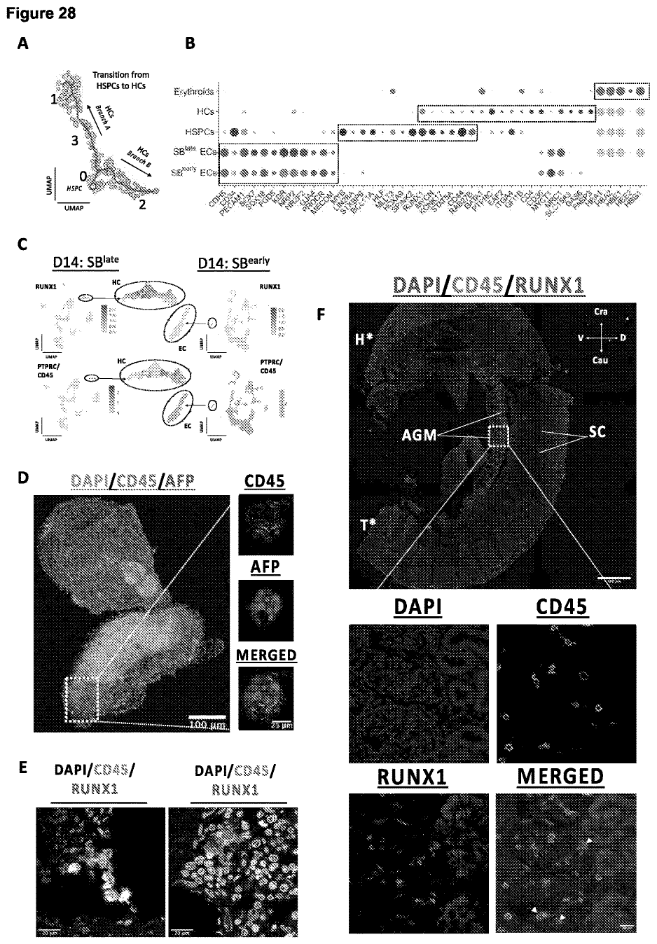

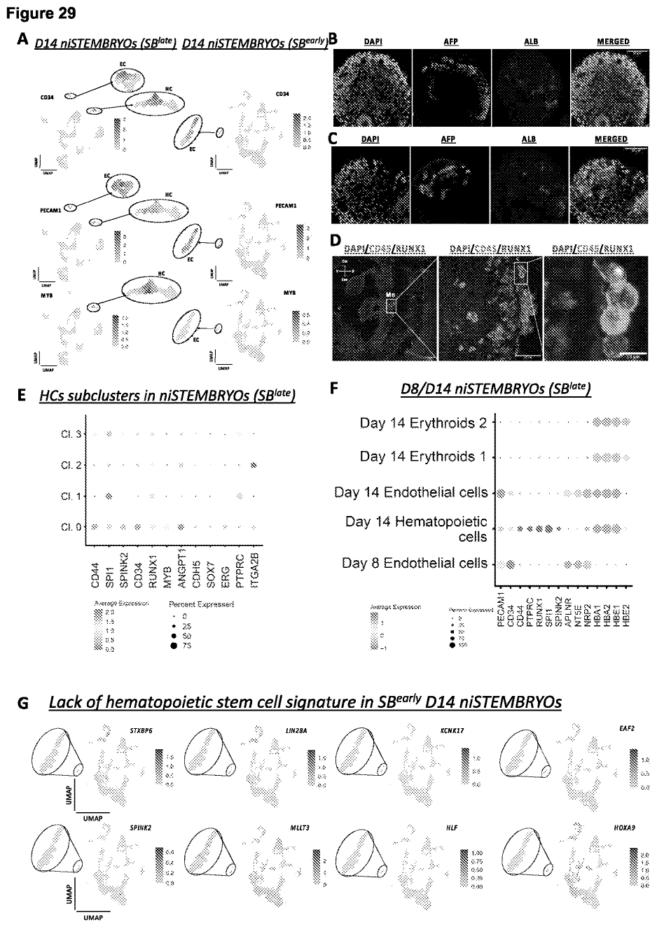

The inventors set out to induce a transient change in cellular states from pluripotent human embryonic stem cells (hESCs) with the potential to develop into three germ-layer derivatives and human primordial germ cell-like cells (hPGCLCs). Previous work by Moris and colleagues¹¹ demonstrated elongation of dissociated and reaggregated cells pulsed with Wnt agonist CHIRON (CH), which also induces primitive streak (PS) formation²⁰. However, since Nodal induces definitive endoderm (DE) in hPSCs^{21,22}, hESCs were dissociated into single cells and cultured them for 24 h with CH and Nodal agonist ACTIVIN-A (ACT) (FIG. 1A). The transiently induced cells showed expression of BRACHYURY (BRA or TBXT), EOMES and SOX17 (FIG. 1B). The expression of pluripotency genes, POU5F1 (OCT4), NANOG and SOX2 continued as in pluripotent hESCs (FIG. 1B; FIG. 2A), but a lack of GATA3 and phosphor-(p) SMAD1/5/8, suggests the absence of BMP4 signalling.

Since high Wnt activity can prevent hPGCLC induction in favour of the endoderm lineage^{19,23}, Inventors assessed the developmental capacity of these structures with reduced CH¹¹. As anticipated¹¹ aggregates failed to extend in the absence of CH (designated as 0.00 μM in FIG. 2B). Yet, reduced dosages of CH (0.25 μM, 0.5 μM and 1.0 μM) were sufficient to promote self-organization into 3D elongated structures, which are referred to as hEOs (FIG. 2B). Inventors selected the lowest dose of 0.25 μM CH to perform all further experiments. Inventors observed cell aggregates as early as 3 hours (3 h) after seeding, which developed into ovoid structures by Day (D) 1 (FIG. 2C). After two days, more than 90% (1620/1800, N=10 experiments) of the aggregates elongated along the longitudinal axis (FIG. 2D).

To track the development and progression of the three germ layers in hEOs over eight days, inventors used RUES2-GLR reporter hESCs24, with SOX2-mCitrine, TBXT-mCerulean and SOX17-tdTomato. As early as D2, inventors observed self-organized 3D structures with SOX2, TBXT and SOX17 reporter expression, representing ectoderm, mesoderm, and endoderm, respectively (FIG. 1C; FIG. 2D); these were displayed as distinct groups of cells in different and partially overlapping domains (FIG. 1C-D). Immunofluorescence (IF) analysis of the hEO sections confirmed the expression of SOX2, TBXT and SOX17, and the establishment of three germ layer derivatives in hEOs (FIG. 1E-F). Inventors obtained similar results using W15-NANOS3-tdTomato hESC line, indicating the protocol's robustness (FIG. 1C, D, E; FIG. 2D). Our observations differ from human gastruloids reported previously¹¹, where the expression of these markers was seen at the same pole throughout their development (72-96 h).

### Transcriptome Analysis at Single-Cell Resolution

For further insights into the organisation, dynamics and cellular complexity of the developing hEOs, inventors performed single-cell RNA sequencing (scRNAseq), sampled at D0, D2, D3, D4 and D8. (FIG. 3A; FIG. 4). Inventors identified 22 major cell populations (FIG. 3B), with the progressive emergence of diverse cell types along the sampled time points (FIG. 3C; FIG. 5A-B). D0 represented the transient stage at 24 h in the presence of ACT and CH (FIG. 1A), marking the start of exit from pluripotency (FIG. 1B, FIG. 2A), and the onset of differentiation (FIG. 5C). The transiently induced cells (D0) contained a cluster with ectodermal markers; CRABP2 and SOX1 (FIG. 5D), and within D0 primitive streak (PS) cluster, markers of anterior PS (APS), including SOX17 and FOXC1 (FIG. 5E), and cells with the expression of posterior PS (PPS), including EVX1 and TBX6 (FIG. 5F). The heterogeneous expression of pluripotency and PS markers (FIG. 1B; FIG. 5C, 5E-G) on D0 indicated a transient state following exit from pluripotency and the onset of differentiation. From D2 onwards, the self-organisation of hEOs showed the emergence of different germ layers, with the expression of gastrulation and early neurulation markers. (FIG. 3B-C; FIG. 5A-B).

Cells in the hEOs primarily represented the PS (FIG. 6A-B), mesoderm (FIG. 6D), endoderm (FIG. 7A), and ectoderm (FIG. 7C). Mesodermal cells were further classified into Early, Intermediate, Paraxial, Somitic, and cardiac mesoderm (FIG. 6C). Ectodermal cells were sub-classified into the neural tube, neural crest, and neurons (FIG. 7B). To stage hEOs development against an in vivo counterpart, inventors mapped the transcriptome of hEOs with a rare authentic CS7 human gastrula²⁵ (FIG. 8A), and found that D3 hEOs represented the transcriptional profile of CS7 human gastrula (FIG. 3D-F). In the mixed mesoderm clusters, there was an expression of genes for the extraembryonic mesenchyme (ExE-mes), such as COL3A1, PITX1, LUM, ETS1, and PCOLCE that is similar to YS mesoderm in CS7 human gastrula²⁵ (FIG. 8B).

To further compare CS7 gastrula and our D3 hEOs, inventors identified differentially expressed genes along the developmental trajectory between the PS and Nascent Mesoderm clusters and classified them based on their patterns of expression (FIG. 8C), which suggests a similar developmental trajectory. The overall comparisons between hEOs, CS7 human gastrula²⁵, and previously described human gastruloids¹¹ (FIG. 3G; FIG. 8D-F) showed the hEOs with neuronal cells, neural crest precursors, neuromesodermal progenitors (NMPs) and hPGCLCs, represents development beyond the CS7 gastrula²⁵, and human gastruloids¹¹. Similarly, comparison of our dataset with CS8-CS11 non-human primate (NHP) embryos²⁶ suggests D8 hEOs with cell types observed in CS11 NHP embryos (FIG. 3H; FIG. 9A), which represents the onset of organogenesis as in CS11 NHP embryos²⁶. Note that the hEOs lacked extra-embryonic lineages, including yolk-sac endoderm and visceral endoderm.

### Rostral-Caudal Axis and NMP Formation

The human body plan develops along the rostro-caudal axis at the onset of gastrulation, with TBXT and CDX2 expression in the posterior region¹¹, and GATA6 expression in the anterior region¹¹. In the hEOs, inventors observed GATA6⁺ cells at one pole and cells with TBXT⁺ or CDX2⁺ expression at the opposite pole (FIG. 9B), consistent with an elongation and differential cell fates along the rostro-caudal axis. Using the IF staining as a guide, inventors could infer the rostral and caudal location of the cell types inventors identified in our dataset (FIG. 9C). In the verification of rostral (GATA6) and caudal (TBXT, CDX2) gene expression, inventors found TBXT and CDX2 expression predominantly in the advanced mesoderm and mixed mesoderm clusters, whereas GATA6 expression was in the cardiomyocytes and mixed mesoderm clusters (FIG. 9C).

Comparing the recently described scRNA-seq data from a human gastrula, the transcriptomic signature of rostral-caudal mesoderm at CS7 was identified²⁵. To verify whether a compatible rostral-caudal pattern was preserved in the hEOs, inventors considered the cells in the intermediate and the mixed mesoderm clusters at D3 (the hEO stage closest to CS7, see FIG. 3D-F). Inventors classified them as rostral and caudal by training the Seurat classifier on the human gastrula data. Nearly all cells in the intermediate mesoderm and ~50% of cells in the mixed mesoderm were classified as “caudal” (FIG. 9D-F). “Caudal” and “rostral” cells show a clear separation in a diffusion map (FIG. 8E-F), suggesting strong transcriptional differences between the two groups of cells. Furthermore, there were transcriptional differences related to rostral and caudal genes within these clusters (FIG. 10A-B), suggesting an elongation of the hEOs along a rostro-caudal axis.

Next, inventors sought neuromesodermal progenitors (NMP) in the caudal region of an embryo, with co-expression of a neural factor, SOX2, and a mesodermal factor Brachyury (or TBXT); NMPs differentiate into neural ectoderm and paraxial mesoderm. In our scRNA-seq dataset, inventors observed the expression of markers for NMP from as early as D2 (FIG. 3B; FIG. 5A-B), including TBXT, SOX2 and NKX1-2 (FIG. 10C). Inventors verified the presence of NMP in hEOs with a reporter cell line showing the co-expression of SOX2 and TBXT (FIG. 10D), and by IF using two different cell lines, RUES2-GLR and W15-Namos3-tdTomato (FIG. 10E). Our scRNAseq dataset detected TBXT⁺SOX2⁺, SOX2⁺NKX1-2⁺ and TBXT⁺NKX1-2⁺ cells on D2-D4, confirming the presence of NMP in the hEOs (FIG. 10F).

NMPs originate at the anterior primitive streak of mammalian embryos²⁷ and contribute to both neural and mesodermal progenitors²⁸. Here, inventors investigated the expression of neuronal precursors, which was not detectable until D3 in hEOs, consistent with CS7 human gastrula²⁵ and human gastruloids¹¹ (FIG. 3B, D-G). Indeed, on D4, expression of neural crest markers, including S100B and SOX10 (FIG. 11A, 11D) and neuronal markers, including ELAVL3 and NEUROD4 (FIG. 11B, 11E), was detected. Furthermore, on D8, inventors detected derivatives of neuroectodermal lineage, including the neural crest (TFAP2A and TFAP2B) (FIG. 11A, 11F), neurons (STMN2 and ASCL1 (FIG. 11B, 11G), neural tube (SOX3 and PAX6) (FIG. 7B, FIG. 12A) and spinal cord precursors (OLIG3 and SOX1,) (FIG. 11C. FIG. 12B). Inventors confirmed the expression of SOX1⁺ and PAX6⁺ neuronal ectoderm (FIGS. 12C, 12E) and SOX2⁺, TFAP2A⁺ neural crest cells by IF on D8 (FIG. 12D).

To find the precursors of neuronal lineage, inventors applied the Waddington Optimal Transport (WOT) algorithm²⁹, mapping the precursors of neural crest, spinal cord, and neural tube clusters at D8 onto the datasets on previous days. This analysis revealed the clusters at D2-D4 that most likely represented the precursor states of the neuronal clusters found on D8 (FIG. 12F). Inventors found that the neural crest, spinal cord and neural tube clusters were linked to NMP and Ectoderm at earlier time points, suggesting the contribution of Ectoderm and NMP in forming neuronal precursors in the hEOs.

Detection of PGCLCs in hEOs

Several studies have shown how hESCs gain competence for germ cell fate and the critical regulators of hPGCLCs.^{19,30,31}. Notably, there are mechanistic differences between rodents and most other mammalian species concerning PGC specification^{18,32-34}, which is attributed to the differences in post-implantation development; egg cylinders in rodents versus bilaminar discs in most mammals^{19,33,34} While the precise in vitro approaches differ³⁵⁻³⁹, exogenous BMP2 or BMP4 is essential for the induction of hPGCLCs^{23,30,31,40,41}. Here, in the transiently induced cells (D0), inventors found the expression of EOMES and TBXT, suggesting their potential competence for germ cell fate (FIG. 1B; FIG. 5C). Inventors set out to investigate the specification of PGCLCs in the hEOs using an hESC line with a NANOS3-tdTomato reporter, a highly specific hPGC marker¹⁹.

First, inventors investigated the optimum dosage of the Wnt agonist, CH, required for the elongation of hEOs along the rostro-caudal axis that is compatible with the hPGCLC specification¹⁹. No hPGCLCs were detected in the absence of CH (0.0 μM), or with a high concentration of CH (1.0 μM) (FIG. 13A). However, with a CH concentration of 0.25 μM, inventors observed both the elongation of hEOs and the specification of hPGCLCs as judged by detecting NANOS3-tdTomato reporter from D2, which continued until D8 (FIG. 14A; FIG. 13B). Strikingly, the specification of hPGCLCs occurred without exogenous BMP2/4, unlike other in vitro models^{19,30,31,42}. Inventors performed IF on the hEO sections on D2, D3, D4 and D8 using a combination of established PGC markers and observed the co-expression of NANOS3⁺SOX17⁺TFAP2C⁺, TFAP2C⁺SOX17⁺POU5F1⁺ and PRDM1⁺/BLIMP1⁺POU5F1⁺ (FIG. 14B-C; FIG. 13C-D). The hPGCLCs were located at the posterior midline of the hEOs in the proximity of TBXT⁺ mesoderm/PS cells. (FIG. 14B-D).

Next, inventors examined scRNA-seq datasets from D2 and D3 hEOs to detect putative hPGCLCs. Due to the rarity of hPGCLCs (estimated to be the region of ~10043), and the potential heterogeneity of gene expression in nascent PGCLCs, inventors focused on triple-positive cells for a combination of important PGC markers; NANOS3, POU5F1, PDPN, TFAP2C, PRDM1, PRDM14, CD38 and NANOG. Using these criteria, inventors identified 61 putative hPGCLCs in the PS and the amnion-like cell (AMLC) clusters (FIG. 14E-F, Table 1), which prompted an investigation of their association with the two cell types. Inventors sub-classified PS and AMLC clusters and re-clustered them as PS, AMLC, Early mesoderm, Ectoderm and NMP from D2 and D3 hEOs (FIG. 13E).

Notably, hPGCLCs in D2 were detected in the AMLCs and the PS, whereas on D3, hPGCLCs were detected only in the PS (FIG. 13F-G). Evidence shows TFAP2A expression in the precursors that contribute to both the hPGCLCs and the AMLC; the loss of TFAP2A and gain of TFAP2C follows in hPGCLCs in the PS¹⁸. In our dataset, PGCLCs on D2 showed the co-expression of TFAP2A and TFAP2C but not in D3 PGCLCs, where inventors detected TFAP2C (FIG. 13F-H). Recent evidence suggests that the PGCs and the amnion share TFAP2A⁺ precursors^{18,41}; this may account for our observations in hEOs and the location of some PGCs in the amnion in monkeys³². Inventors performed IF on D2 hEOs and observed cells with the co-expression of SOX17 and ISL1⁴⁴ (FIG. 14G), confirming the presence of AMLCs.

To detect a potential source of endogenous BMP4 for the specification of PGCs⁴⁵, inventors examined the scRNA dataset. Inventors found a higher enrichment of BMP4 in AMLC compared to PGCLCs and PS (FIG. 14F). Conversely, BMP receptor type 1A (BMPR1A) was enriched in PGCLCs and PS only, while BMP receptor type 2 (BMPR2) expression was enriched in AMLC; this is consistent with CS7 human gastrula, with the expression of BMP4 and its receptor, BMPR2, in amniotic cells (previously labelled as non-neural ectoderm²⁵), suggesting AMLC as a likely source of BMP4. Notably, the inhibition of BMP by LDN193189 affected the specification of hPGCLCs when present on D1 but not on D2 or D3 (FIG. 14H).

### Post-Gastrulation Development

A significant limitation of existing models, such as gastruloids, is that they start to retract after three days in static culture¹¹. To overcome this limitation, inventors explored methods to extend development by combining static with kinetic culture using a rotary system^{8,46}. To scale up the generation of hEOs from 96 to 1200, inventors transitioned from 96 well plates to Aggrewell™ 400 plates for initial static cultures to harness the potential throughput offered by rotary cultures. Scaled hEOs were cultured for four days in the presence of CH (FIG. 15A), resulting in the elongation of ~60% of the aggregates by day 4 (1845/3100; N=4 experiments), exhibiting three germ layers (FIG. 16A), NMPs (FIG. 16B) and hPGCLCs (FIG. 16C), consistent with their development in 96-well plates.

Visibly elongated hEOs were selected (N=~500 hEOs) on D4 for transfer to a hypoxic (5% O2, 5% CO2) rotary system using the same culture media (FIG. 15A). However, the aggregates adapted poorly and failed to grow or display consistent expression of transcription factors (FIG. 16D). Inventors attempted several other approaches, including the use of ex-utero culture medium (EUCM2)⁸, the addition of TGF agonist SB431542 (SB43 or SB; 1 μm), supplementation with human cord serum (HCS). Inventors also attempted culture in a solidified extra-cellular matrix (Collagen type 1) in the static cultures before transferring and adding a soluble laminin-rich matrix in the rotary cultures (10% Matrigel®). The addition of Collagen to static cultures caused the webbing of hEOs into poorly organised structures that were not suited for transfer to the kinetic cultures (FIG. 16E). Likewise, supplementation with HCS and EUCM2 resulted in highly irregular and small structures without discernible or reproducible reporter expression patterns (FIG. 16E).

However, inventors found that the addition of SB43, a small molecule inhibitor of the TGF-ß pathway⁴⁷, known to enhance the proliferation of cytotrophoblast cells⁴⁸, and generation of haematopoietic progenitors⁴⁹, had a striking effect on hEOs development in the kinetic culture (FIG. 15B-C), with or without supplementation with 10% soluble Matrigel; consequently, the hEOs doubled in size after four days (FIG. 15B-C; FIG. 16F-G). The subsequent experiments were performed with only SB to reduce potential sources of variability and costs. Intriguingly, at D7 in kinetic culture with SB, inventors observed periodic and highly localised contractions of cells in ~10% of hEOs, which increased to almost 70% by D8 (FIG. 4D). The periodic contractions were not evident in hEOs in cultures with HCS or EUCM2. Indeed, adding HCS abrogated the periodic contractions even in the presence of SB (FIG. 15D). To assess whether contractions were due to the development of a beating heart, inventors performed wholemount IF staining of the Cardiac Troponin Type 2 (TNNT2) and myosin heavy chain 2 (MYH2) (FIG. 15E-F). Expression of these cardiomyocyte-specific markers was localised to the anterior region of hEOs and appeared to pattern a tubular structure reminiscent of a primitive heart tube (PHT) (FIG. 15E-F). During embryogenesis, the first heartbeat begins in the PHT at the gestational day 22⁵⁰, before the PHT forms a loop and develops into a chambered heart 51. These results suggest that hEOs present contractile cardiomyocytes, representative of PHT, before developing a chambered heart.

At this time, there is also the initiation of gut tube formation seen as SOX17⁺ tubular structures at the other end of ‘beating’ hEO regions on D8, suggestive of an early gut tube (FIG. 16H). Adjacent to the SOX17⁺ gut tube was a SOX2⁺ tubular structure, suggestive of a neural ectoderm. Further analysis of the SOX2⁺ neuronal cells showed a tubular structure, indicating the presence of a neural tube (FIG. 16I). The GATA4⁺ cells clustered below the neural tube-like structure shows the cardiomyocytes. In contrast, the scattered SOX17⁺ cells could be endothelial precursors^{7,52}. The hEOs also expressed SOX9⁺ cells adjacent to the SOX17⁺ cells, suggesting the putative neural crest cells (FIG. 16J). Hence, the hEOs in kinetic culture supplemented with SB display multifaceted features representing post-gastrulation human embryo development.

To further evaluate the impact of SB, inventors extended the duration of SB supplementation to the initial four-day static cultures. Inventors observed a significant increase (by 15%, p=0.0016) in the frequency of elongated hEOs on D4 (FIG. 15G). However, transferring these hEOs to the kinetic rotary cultures resulted in a significant decrease (by 40%, p<0.0001) in the number of ‘beating’ hEOs on D8, compared to those where SB was present only during the kinetic cultures (FIG. 15H).

Inventors next sought to extend kinetic cultures of hEOs and found that after 22 days, hEOs cultured with SB quadrupled in size relative to the size in static culture on D4 (FIG. 16K). However, the number of ‘beating’ hEOs decreased between D14 and D22, and none displayed discernible contractions by D22. Instead, from day 14 onwards, hEOs began to develop visible red inclusions, with ~14% of hEOs containing large internal red zones by day 15 (FIG. 15I); the appearance of these red inclusions depended on SB supplementation to rotary cultures (FIG. 15J). However, this phenotype was entirely lost when SB was included in the 4D static culture, suggesting a time-dependent role of SB in the maturation of these structures. The striking red phenotype suggested a potential initiation of early fetal haematopoiesis in the hEOs. Accordingly, inventors performed IF staining for hemogenic progenitors and observed RUNX1⁺ hemogenic endothelial progenitors in D14 hEOs (FIG. 15K; FIG. 17A). RUNX1 is required for endothelial to haematopoietic cell transition⁵³, whereby individual cells bud off and detach from the endothelial layer⁵⁴ and begin expressing the major transmembrane glycoprotein CD45, the bona fide marker of blood formation. Indeed, inventors observed membrane localisation of CD45 both in elongated RUNX1⁺ cells and in spread out RUNX1 cells adjacent to Runx1⁺CD45⁻ endothelial sites, suggesting that hEOs capture this critical transition in early human haematopoiesis (FIG. 15K-L; FIG. 17A).

To establish an explicit link between visible red inclusions with a hemogenic phenotype, inventors split hEOs into groups, with (control) or without (red) putative hemogenic zones and dissociated them for quantitative flow cytometric analysis. There were significantly more DAPI⁻, CD45⁺ live hematopoietic cells in the red hEOs than in controls when gated against a technical fluorescent minus one (FMO) PE Cy7 control (FIG. 17C; FIG. 15M); the red hEOs contain approximately 4-5% hematopoietic cells (FIG. 15N). The CD45⁺ population showed expression of progenitor marker c-KIT as well as gradient loss of expression of endothelial marker CD31 (FIG. 15O), in line with inferred endothelial-to-hematopoietic transition observed with loss of RUNX1 captured by IF (FIG. 15K-L), Finally, a distinct population expressing epithelial cell adhesion molecule EpCAM was visible in both CD45⁻ and CD45⁺ populations, and while the former delineates epithelial cells, expression of EpCAM within CD45⁺ immune cells is indicative of the presence of erythroid progenitors^{55,56}, a putative source of the red pigmentation visible within the hEOs (FIG. 15H; FIG. 17B). These results suggest that the formation of a beating heart tube in hEOs is followed by the emergence of RUNX1⁺ hemogenic progenitors and CD45⁺ HPCs, thus confirming the occurrence of fetal haematopoiesis in the hEOs.

### CONCLUSIONS

Here, inventors show that transiently induced hESCs self-organise into 3D hEOs, which elongate along a rostro-caudal axis, forming derivatives of the three germ layers, and display hPGCLCs in the absence of exogenous BMP. Our analysis suggests AMLCs as the source of endogenous BMP required for the induction of PGCLCs. Furthermore, the co-expression of hPGCLCs with AMLC suggests their co-localization during early specification or shared progenitors. The strong alignment of D3 hEOs with CS7 human gastrula suggests a credible representation of in vivo human development. The hEOs have the potential to develop very significantly, capturing key aspects of post-gastrulation development. The emergence of NMP and subsequent neuronal progenitors, beating heart tube, primitive gut, and haematopoiesis reveal post-gastrulation features in developing hEOs, establishing a credible foundation for the study of early post-implantation human embryo development in vitro. The similarity of D3-D4 hEOs with the previously described human gastruloids cultured over 72-96 h¹¹, shows the significant advancement of the hEOs during the additional development period of up to 22 days. Notably, the emergence of haematopoietic cells with a red pigmentation, evidently in the absence of the extra-embryonic yolk-sac, suggests the initiation of fetal endothelial-to-hematopoiesis transition in hEOs.

Note that there is an evident lack of a yolk sac and most other extraembryonic tissues in the hEO model, which could affect aspects that might influence the development of human embryos in vivo. Nevertheless, the versatility and potential scalability of hEO cultures can provide a basis for investigations for modelling some aspects of human development and disease. Similarly, therapeutic and environmental factors can be screened for the potential adverse impact on early post-implantation human development.

### Materials and Methods

Human Embryonic Stem (hES) Cells Culture and Induction of a Transient State

Inventors used W15-NANOS3-tdTomato and RUES2-GLR hES cell lines in this study. All hES cells were cultured in Essential 8™ (E8) medium (Life Technologies, A1517001) on freshly prepared Vitronectin (Fisher Scientific, A31804) coated 6-well plates in humidified chambers at 37° C. and 5% CO2. hES cells were passaged every 3-4 days (once they reached ~70% confluency) using 0.5 mM EDTA (ThermoFisher Scientific, AM9260G) in homemade phosphate buffer saline (1×PBS). The transient state of cells was induced, as described previously, with slight modifications¹⁹. In brief, hES cell colonies (70% confluent) were dissociated into single cells using 0.25% Trypsin-EDTA (Life Technologies, 25200072), seeded (600,000 cells/well) into freshly prepared Vitronectin-coated 6-well plates (~30 mins). The cells were cultured for 24 h in the presence of Advanced RPMI 1640 (Life Technologies, 61870-036) supplemented with B27 supplement (1%) (ThermoFisher Scientific, 17504044), 0.1 mM NEAA, 100 U/ml Penicillin, 0.1 mg/ml Streptomycin, 2 mM L-glutamine, Activin A (1 μg/ml) (In-house produced by Biochemistry Department, University of Cambridge), Chiron (3 μM) (CHIR99021, TOCRIS 4423/10) and Rho-associated, coiled-coil containing protein kinase (ROCK) inhibitor (Rocki: 10 μM); Y27632) (hereafter called induction medium, IM).

Self-Organization of hES Cells into 3D hEOs

Transient cells induced in IM for 24 h were dissociated into single cells using 0.25% Trypsin-EDTA and seeded (400 cells/well) into Corning Clear Ultra-low attachment 96-well plates (Fisher Scientific 10023683). 100 μl/well of Essential 6 (E6) medium (Life Technologies, A1516401) supplemented with 0.25 μM CH, and ROCKi (10 μM) was used for self-organization of the intermediate cells (hereafter termed as organization medium, OM). 96-well plates with singly dissociated cells were centrifuged at 1200 rpm for 5 mins to let the cells settle at the centre-bottom of each well. Peripheral wells of the 96-well plates were filled with the same volume of 1×PBS to avoid evaporation of the culture medium. PBS was added in peripheral wells of the plate also to prevent the scattering of cells in outer wells even after centrifugation that would otherwise form satellite aggregates instead of forming a single aggregate during self-organization. Inventors started with 0.25 μM CH for 100 μl OM per well for the first three days of self-organization. The concentration of CH was reduced progressively starting on D3, when 100 μl E6 medium was added to each well, resulting in approximately 0.125 μM CH in a volume of 200 μl OM; the hEOs were cultured for the next three days. On D6, 100 μl of the OM was removed from each well, and 100 μl E6 medium was added, resulting in approximately 0.06 μM CH in the final 200 μl OM. After that, hEOs were cultured for additional two days in this medium.

Immunofluorescence of hESCs and Transiently Induced Cells

hESCs and transiently induced cells were cultured on Ibidi 8-well-slides (Thistle Scientific LTD., IB-80826). Transiently induced cells cultured for 24 h were washed in 1×PBS and fixed in 4% PFA for 10 min at room temperature, whereas hESCs were cultured for 3-4 days before fixation. Samples were incubated with primary antibodies overnight at 4° C. and with fluorescently conjugated secondary antibodies for 1 h at room temperature. Samples were stained with DAPI (Sigma, D9542-1 MG) to mark nuclei and were observed under confocal laser scanning microscopy. Antibodies used are listed in Table 2.

TABLE 2
List of antibodies used.

| | | | Reference |
|---|---|---|---|
| Antibodies | Host | Supplier | Number |
| POU5F1 (OCT4) | Mouse | BD Biosciences | 611203 |
| NANOG | Rabbit | Peprotech | 500-P236 |
| SOX2 | Mouse | Santa Cruz | Sc3465823 |
| SOX2 | Goat | Abcam | Ab239218 |
| SOX2 | Goat | Santa Cruz | Sc17320 |
| TBXT (BRA/T) | Rabbit | Abcam | Ab209695 |
| SOX17 | Goat | R & D Systems | AF1924 |
| SOX17 | Rabbit | Cell Signalling | 817785 |
| | | Technology | |
| PRDM1 (BUMP1) | Rabbit | Cell Signalling | 9115 |
| | | Technology | |
| TFAP2C (AP-2Y) | Rabbit | Abcam | Ab218107 |
| TFAP2A (AP2a) | Mouse | Santa Cruz | Sc32762 |
| EOMES | Sheep | R & D Systems | AF6166 |
| SOX1 | Goat | R & D Systems | AF3369 |
| GATA6 | Goat | R & D Systems | AF1700 |
| CDX2 | Rabbit | Cell Signalling | 123065 |
| | | Technology | |
| SOX1 | Goat | R & D Systems | AF3369 |
| RUNX1 | Mouse | Insight Biotechnology | sc365644 |
| RUNX1 | Rabbit | Abcam | ab23980 |
| CD45 | Mouse | BD Biosciences | 555480 |
| MHC-II | Mouse | R & D Systems | MAB4470 |
| TNNT2 | Rabbit | Abcam | ab209813 |
| RFP | Mouse | Rockland/Cambridge | 200-301-3795 |
| | | Bioscience | |

### Cryo-Sectioning and Immunofluorescence (IF)

hEOs cultured on ultra-low attachment 96-well plates were collected, washed in 1×PBS and fixed in 4% PFA for 3-4 hours at room temperature or 1% PFA overnight at 4° C. hEOs were embedded in OCT mounting medium (VWR-361603E) and incubated in dry ice for 30 min before sectioning the frozen samples. An antigen retrieval procedure was performed to remove the fluorescence reporter from W15-NANOS3-tdTomato hES cell line, where required. In brief, cryosectioned slides were incubated at room temperature for 30 minutes and boiled in TE buffer (pH 6.0) for 30 minutes in a microwave oven. Cryosectioned slides were dried up at room temperature for 45 minutes before performing IF. Anti-RFP primary antibody was used to visualise NANOS3 protein in W15-NANOS3-tdTomato hES cell line, where required. Antigen retrieval was performed to remove the endogenous tagging where needed. All samples were incubated with primary antibodies overnight at 4° C. and with fluorescently conjugated secondary antibodies for 1 h at room temperature. Samples were stained with DAPI (Sigma, D9542-1MG) to mark nuclei and were observed under confocal laser scanning microscopy. Antibodies used are listed in Table 2.

### Image Analysis

IF was followed by confocal microscopy (Leica SP8 and SP5 inverted microscope) for imaging. Image analysis was carried out by image processing package, Fiji 2⁵⁷.

### 10× Genomics

For each stage, 20 hEO were collected and dissociated into single cells using 0.5% Trypsin-EDTA. Singly dissociated cells were collected into an Eppendorf tube containing PBS with 0.04% weight/volume BSA (400 μg/mL). Cells were filtered using 50 μM disposable filters (CellTrics™) and counted with an automatic cell counter (LUNA™ Dual fluorescence cell counter). Cells were loaded into the 10×-Genomics Chromium using the single cell 3′ reagents kit v3. Libraries were prepared as per the manufacturer's instructions and pooled for sequencing. Libraries were sequenced on an Novaseq 6000. Sequencing was performed in biological replicates for each stage derived from independent experiments.

### Single-Cell RNA Sequencing Analysis

### Data Alignment and Quality Control

The reference genome was generated with the command mkref from cellranger (version 4.0.0), using the pre-built human genome (GRCh38). Starting from fastq files, the raw count matrix was obtained with the command count from cellranger 4.0.0 for each batch (there are 2 batches for each day). Quality control analysis was performed on the raw count matrix, keeping only cells with a number of expressed genes greater or equal to 1000 and fraction of mitochondrial genes less than or equal to 0.20. Inventors ran SoupX⁵⁸ on the count matrix to correct for potential environment RNA contamination (function autoEstCont from the SoupX library, parameter tfidfMin=0.8).

### Clustering Analysis

After normalization with the function NormalizeData (Seurat version 4.0.5)⁵⁹ and batch effect correction with the FindIntegrationAnchors function, the cluster analysis was performed with the FindNeighbors (k.param=20) and FindClusters functions on the top 20 PCA components computed from top 2000 genes selected with SelectIntegrationFeatures. The resolution value was 0.1 for day 0, day 2, day 3 and day 4 and 0.2 for day 8. Normalization and cluster analysis were done independently for each time point.

To have better sensitivity towards small clusters, inventors refined the clustering analysis at each time point using an approach similar to the one presented earlier⁶⁰. In brief, inventors performed a gene filtering to select all genes that tend to be detected in small cell neighborhoods. To this aim, first, inventors only considered genes with more than 1 normalized counts in more than 10 cells. Then, for each gene, the probability of being detected or not detected in a local region was considered. A local region is a set defined by a cell and its k-nearest neighbours. For a given gene in a given local region, inventors computed the Shannon entropy (with the function Entropy from the R library DescTools version 0.99.44). The entropy was computed for each gene in the local regions defined from 100 randomly selected cells. The entropy of mixing was defined as the mean value of the Shannon's entropy computed across all the local regions. This procedure was repeated 10 times, and the mean of the 10 values of entropy of mixing was computed. Finally, genes were sorted by increasing mean values of entropy of mixing. The genes with a higher ranking correspond to those that were expressed by a relatively small number of cells having similar transcriptomic profiles. Hence, such genes could be markers of rare cell types.

Inventors repeated the clustering at each time point with the top 1000 genes selected with this criterion and retained every new cluster of cells with fewer than 100 cells. Moreover, starting from the original partition, inventors performed a sub-cluster analysis in all the clusters whose top 100 highly variable genes were enriched among genes with entropy of mixing below 0.2. The statistical enrichment was verified with a Fisher's test (R function fisher.test with default parameters, p-value<1e-4) using a background set of all the genes for which the entropy of mixing was calculated. This analysis resulted in one additional cluster at day 2 (annotated as paraxial mesoderm) and two additional clusters at day 8 (annotated as endoderm and axial mesoderm). A customized R script with the analysis based on entropy is available on the GitHub repository https://github.com/ScialdoneLab/Entropy-Mixing.

Inventors identified marker genes for each cluster with the function FindMarkers (with default parameters and only.pos=T). Only markers with adjusted p-value (based on the Bonferroni correction) below or equal to 0.05 were considered for downstream analysis. Finally, genes that were selected as markers for two or more clusters were removed. The list of marker genes was used to perform cluster annotation at each time point.

Inventors noticed that the cluster annotated as “Primitive Streak” at day 3 included cells that co-expressed several NMP marker genes (e.g, NKX1-2, HOXB8, HOXC9). Hence, inventors performed a sub-cluster analysis with the functions FindNeighbors (k.param=5) and FindClusters on the top 30 PCA components computed from top 2000 genes selected with FindVariableFeatures. The resolution value was set to 0.1. This allowed us to identify a sub-cluster of cells expressing NMP markers (such as NKX1-2 and HOXB8), which was annotated as “NMP”. The other two sub-clusters were annotated as “Primitive Streak 1” and “Primitive Streak 2” (FIG. 12F).

Quality control metrics for all the clusters at a given time point are shown with ggplot function from R library ggplot2 (FIG. 4). The quality control metrics represented are: number of UMI counts, number of genes expressed above 0, fraction of mitochondrial reads and fraction of ribosomial reads. Clusters that showed small UMI counts or expressed genes and/or high levels of mitochondrial/ribosomial genes and the absence of clear markers to interpret were labelled as “bad quality” and were excluded from all the downstream analysis. “Bad quality” clusters were present at day 0, day 2, day 3 and day 4. In order to visualize the dataset from a given time point, UMAP coordinates were computed starting from the top 20 PCA components with the function RunUMAP (Seurat version 4.0.5).

### Batch Correction and Data Visualization

For a joint visualization of the data from all the days (after excluding cluster labelled as bad quality), first, a batch correction between samples from different time points was done with scanorama⁶¹ (function correct_scanpy with return_dimred=True). Batch integration was done using as features of all the genes that were highly variable in at least two different days with scanpy⁶² (version 1.8.0) function pp.highly_variable_genes (with parameter batch_key=‘day’). Then the scanpy functions pp.neighbors (with parameters n_pcs=50 and use_rep=“X_scanorama”) tl.paga (with default parameters) were run. A UMAP plot was then generated with the scanpy functions tl.umap (with parameter init_pos=‘paga’) and pl.umap.

Diffusion maps were generated from specific subsets of clusters in the dataset (FIG. 6B, 6D; FIG. 7A, 7C) using the Scanorama-integrated gene expression data as follows: 1000 highly variable genes were selected using the function FindVariableFeatures from the Seurat library in R. Then, based on these genes, a k-nearest neighbors graph was calculated using the function pp.neighbors on 15 principal components specifying that Scanorama-integrated data should be used (n_pcs=15, use_rep=“X_scanorama”). Finally, the diffusion map was generated on the same 15 components with the function tl.diffmap with parameter n_comps=15.

Mapping the Human Embryo Organoid Data onto the Data from a CS7 Human Gastrula, Human Gastruloids and Cynomolgus Monkey

To find the day of organoid development that was closest to a human gastrulating embryo, inventors projected our data onto a recently published scRNA-seq dataset from a CS7 human embryo²⁵. Inventors took the top 2000 highly variable genes in the CS7 dataset selected with the function “FindVariableFeatures” from the Seurat R package using default parameters. Inventors found anchor genes between the CS7 dataset and each of our organoid datasets using the function “FindTransferAnchors” from the Seurat R library with 50 principal components and parameters k.anchor=100 and k.filter=500. Finally, to transfer the labels from the CS7 dataset to our organoids cells, inventors employed the function “TransferData” using 30 principal components and parameter k.weight=100. This procedure provided us with “cell type scores” for all cells in our organoid dataset, representing how similar their transcriptome was to any of the cell types present in the CS7 human embryo. For each cluster in our hEOs dataset at any given time point, the mean of the maximum score was computed across all cells. Finally, for each time point, the distribution of these mean values across all clusters is shown with the ggplot function from R library ggplot2 (version 3.3.5).

The human 3D gastruloid¹¹ and cynomolgus monkey (CM) 26 data sets were downloaded from GEO (accession numbers: GSE123187, GSE193007). For the gastruloid data, the unique UMI-corrected data sets were scaled and integrated with our hEOs datasets (day 2, day3, and day4) using reciprocal PCA as implemented in Seurat. The CM data sets were integrated with hEOs data sets (day3, day4 and day8), respectively. Due to low cell numbers in the gastruloid datasets, neighborhood parameters were set to k=3 for all functions in Seurat. Heatmaps represent the prediction scores for individual gastruloid or CM cells using the label transfer function (‘TransferData’) in Seurat with hEOs cell types as a reference.

To test if inventors could find a rostral-caudal signal in the mesodermal cells from our embryo organoids, inventors took the clusters from day 3 that resembled the Advanced Mesoderm cluster in the CS7 dataset (based on the cell type scores introduced above), where the transcriptional differences between rostral and caudal genes were strongest⁶⁰. In particular, inventors took the hEOs clusters annotated as Intermediate Mesoderm and Mixed Mesoderm, since most of the cells from them were mapped onto the CS7 Advanced Mesoderm cluster. Inventors trained a Seurat classifier on the caudal versus rostral cells from the CS7 Advanced Mesoderm cluster, and then used it to classify the hEO cells as caudal or rostral. A Wilcoxon Rank-Sum test was used to find differentially expressed genes between the caudal and the rostral cells.

### Mapping Clusters Across Time Points

Inventors used the WOT algorithm⁶³ to find the most likely differentiation trajectories between clusters across time points. For each cell at time t, this algorithm assigned a “bias score” toward every cluster at time t+1 with the function compute_all_transport_maps. For each cell, the sum of the scores towards all the clusters at time t+1 sum to 1. Each cluster of cells at time t gets assigned a score equal to the average scores of all cells in that cluster. Inventors applied this procedure to identify the most likely precursor cells of the clusters spinal cord, neural tube and neural crest at day 8. Finally, inventors visualized the results by showing the connections between each time point for each cluster using the R library WOTPLY¹⁶ based on the R library GGally (version 2.1.2)¹⁷. The thickness of the edges between clusters at time points t and t+1 represents the average score, and only the edges with an average score above 0.2 are shown.

Identification of hPGCLCs in scRNA Dataset

The Primitive Streak, NMP, Ectoderm and Early Mesoderm clusters from Day 2 and Day 3 were integrated using Seurat canonical cluster analysis (CCA). Cells were re-clustered using the Louvain algorithm implemented in Seurat with a resolution of 1.0. Samples were also integrated using Scanorama. One subcluster was identified as AMLC expressing the markers TFAP2A, GATA3, MSX2 and STOM. Sixty-one putative PGCLCs were identified at Day 2 and Day 3 as triple-positive cells (normalized counts >0.1) of key PGC markers among NANOS3, PDPN, POU5F1 (or OCT4), TFAP2C, PRDM1, PRDM14 and CD38. Only potential PGCLC combinations were selected for further analysis (see Table 1). hPGCLCs with co-expression of TFAP2A and TFAP2C (normalized counts >0.1) are highlighted.

### Flow Cytometry

Analysis of hEO by flow cytometry was performed on a SONY SH800 cell sorter. Briefly, hEO were dissociated in 0.25% Trypsin/EDTA (GIBCO) for 10-15 minutes at 37° C., quenched in 10% FCS in PBS, centrifuged for 3 minutes at 1200 RPM, resuspended in 2% FCS in PBS, and filtered through a 70 μM mesh. Cells were then stained with CD45 (PE Cy7), CD31 (APC), c-KIT (PE), and EpCAM (FITC) and DAPI for 30 min at 4° C., with 10 μM Rho-Kinase inhibitor added to the staining cocktail to minimize cell death. Cells were subsequently rinsed and maintained on ice in 2% FCS supplemented with EDTA and HEPES until analysis. Compensation of the panel was performed using UltraComp Beads (Invitrogen) to minimize and correct for spillover between channels. Data was analysed and plots created using FlowJo v10.8.2.

### Data Availability

Raw data for the human embryo organoids are available through ArrayExpress, under accession numbers E-MTAB-12045. (Reviewers login (view only) Username: Reviewer_E-MTAB-12045 Password: fecumciq).

### Post-Gastrulation Development of Human Embryo Organoids

24 well Aggrewell™ 400 plates (StemCell Technologies 34411) were pre-treated with 500 μl/well anti-adherence medium and centrifuged at 1300×g for 5 mins. After aspirating the medium, the wells were rinsed with 2 ml warm basal medium. Then, the medium was replaced by 1 ml warm organisation medium (OM). The plate was incubated at 37° C. at 5% CO₂ until the cells were ready for seeding. OM comprised of Essential 6 (E6) medium (Life Technologies, A1516401) supplemented with 0.25 μM CH and ROCKi (10 μM). Pre-induced cells were dissociated using 0.25% Trypsin-EDTA, and 480,000 cells were seeded into each well of an Aggrewell™ 400 Plate at the rate of 400 cells/microwell and were centrifuged at 300×g for 3 mins. The final volume of medium in each well was 2 ml. Cells were cultured in Aggrewell plates at 37° C. at 5% CO₂ for 4 days. The OM (without ROCKi) was replaced every day. On day 5, hEOs were manually picked and transferred to bottles for rotary culture in a bioreactor. From Day 5, hEOs were cultured in OM supplemented with 1 μM SB431542 at hypoxic conditions (5% O2, 5% CO₂ and without ROCKi). The hEOs were collected at different time points for further analysis.

Medium containing human cord serum (HCS) (also known as ex-utero culture medium, EUCM) was used as described earlier⁶⁴. In brief, 25% human cord serum was mixed with 25% DMEM (Gibco 11880) (low glucose, no phenol red, no glutamine, no HEPES) and 50% rat serum. Glutamine and Penicillin-Streptomycin (50 units/ml penicillin) were added to the medium supplemented with 4 mg/ml glucose and HEPES (GIBCO 15630056, 11 mM) and 1 mM Sodium Pyruvate. Human cord serum was provided by the Cambridge Blood and Stem Cell Biobank, which is supported by the Cambridge NIHR Biomedical Research Centre, Wellcome Trust-MRC Stem Cell Institute, UK. Rat serum was heat-inactivated for 30 min at 56° C. and filtered through 0.22 μm PVDF filter, whereas human serum was heat inactivated at 55° C. for 45 minutes and sterilized through filtration by 0.22 μm filters. Modified in vitro culture (IVC) 65 medium also termed a EUCM28 medium was used as described previously⁷. Briefly, advanced DMEM/F12 was supplemented with 30% (Vol/Vol) FBS, 1 mM Sodium Pyruvate, 1 mg/ml glucose monohydrate, 100 nM T3, 1 mM glutamax, 1×ITS-X, 8 nM β-estradiol, 200 ng/ml progesterone and 25 μM N-acetyl-L-Cysteine. Medium containing HCS or EUCM2 were used from D4 onwards during kinetic culture of hEOs under hypoxic conditions (5% CO2, 5% O2).

### Example 1 References

1 Morgan, L. M. 1 online resource (329 p.) (University of California Press, Berkeley, 2009).
2 Streeter, G. L. Developmental horizons in human embryos; a review of the histogenesis of cartilage and bone. https://doi.org: D-CLML: 4917:194k1 OTO-NLM
3 Rivron, N. C. et al. Blastocyst-like structures generated solely from stem cells. Nature 557, 106-111 (2018). https://doi.org: 10.1038/s41586-018-0051-0
4 Li, R. et al. Generation of Blastocyst-like Structures from Mouse Embryonic and Adult Cell Cultures. Cell 179, 687-702.e618 (2019). https://doi.org: 10.1016/j.cell.2019.09.029
5 Kagawa, H. et al. Human blastoids model blastocyst development and implantation. Nature 601, 600-605 (2022). https://doi.org: 10.1038/s41586-021-04267-8
6 Liu, X. A.-O. et al. Modelling human blastocysts by reprogramming fibroblasts into iBlastoids.
7 Amadei, G. et al. Embryo model completes gastrulation to neurulation and organogenesis. Nature 610, 143-153 (2022). https://doi.org: 10.1038/s41586-022-05246-3
8 Tarazi, S. et al. Post-gastrulation synthetic embryos generated ex utero from mouse naive ESCs. Cell 185, 3290-3306.e3225 (2022). https://doi.org: 10.1016/j.cell.2022.07.028
9 Beccari, L. et al. Multi-axial self-organization properties of mouse embryonic stem cells into gastruloids. Nature 562, 272-276 (2018). https://doi.org: 10.1038/s41586-018-0578-0
10 van den Brink, S. C. et al. Symmetry breaking, germ layer specification and axial organisation in aggregates of mouse embryonic stem cells. Development 141, 4231-4242 (2014). https://doi.org: 10.1242/dev.113001
11 Moris, N. et al. An in vitro model of early anteroposterior organization during human development. Nature 582, 410-415 (2020). https://doi.org: 10.1038/s41586-020-2383-9
12 Simunovic, M. et al. A 3D model of a human epiblast reveals BMP4-driven symmetry breaking. Nat Cell Biol 21, 900-910 (2019). https://doi.org: 10.1038/s41556-019-0349-7
13 Olmsted, Z. T. & Paluh, J. L. Co-development of central and peripheral neurons with trunk mesendoderm in human elongating multi-lineage organized gastruloids. Nature Communications 12, 3020 (2021). https://doi.org: 10.1038/s41467-021-23294-7
14 Warmflash, A., Sorre, B., Etoc, F., Siggia, E. D. & Brivanlou, A. H. A method to recapitulate early embryonic spatial patterning in human embryonic stem cells. Nat Methods 11, 847-854 (2014). https://doi.org: 10.1038/nmeth.3016
15 Minn, K. T. et al. High-resolution transcriptional and morphogenetic profiling of cells from micropatterned human ESC gastruloid cultures. Elife 9 (2020). https://doi.org: 10.7554/eLife.59445
16 Zheng, Y. et al. Controlled modelling of human epiblast and amnion development using stem cells. Nature 573, 421-425 (2019). https://doi.org: 10.1038/s41586-019-1535-2
17 Shao, Y. et al. A pluripotent stem cell-based model for post-implantation human amniotic sac development. Nat Commun 8, 208 (2017). https://doi.org: 10.1038/s41467-017-00236-w
18 Castillo-Venzor, A. et al. Origin and segregation of the human germline. bioRxiv, 2022.2007.2006.498671 (2022). https://doi.org: 10.1101/2022.07.06.498671
19 Kobayashi, T. et al. Principles of early human development and germ cell program from conserved model systems. Nature 546, 416-420 (2017). https://doi.org: 10.1038/nature22812
20 Loh, K. M. et al. Efficient endoderm induction from human pluripotent stem cells by logically directing signals controlling lineage bifurcations. Cell Stem Cell 14, 237-252 (2014). https://doi.org: 10.1016/j.stem.2013.12.007
21 D'Amour, K. A. et al. Efficient differentiation of human embryonic stem cells to definitive endoderm. Nat Biotechnol 23, 1534-1541 (2005). https://doi.org: 10.1038/nbt1163
22 Bernardo, A. S. et al. BRACHYURY and CDX2 mediate BMP-induced differentiation of human and mouse pluripotent stem cells into embryonic and extraembryonic lineages. Cell Stem Cell 9, 144-155 (2011). https://doi.org: 10.1016/j.stem.2011.06.015
23 Kojima, Y. et al. Evolutionarily Distinctive Transcriptional and Signaling Programs Drive Human Germ Cell Lineage Specification from Pluripotent Stem Cells. Cell Stem Cell 21, 517-532.e515 (2017). https://doi.org: 10.1016/j.stem.2017.09.005
24 Martyn, I., Kanno, T. Y., Ruzo, A., Siggia, E. D. & Brivanlou, A. H. Author Correction: Self-organization of a human organizer by combined Wnt and Nodal signalling. Nature 564, E10 (2018). https://doi.org: 10.1038/s41586-018-0583-3
25 Tyser, R. C. V. et al. Single-cell transcriptomic characterization of a gastrulating human embryo. Nature 600, 285-289 (2021). https://doi.org: 10.1038/s41586-021-04158-y
26 Zhai, J. et al. Primate gastrulation and early organogenesis at single-cell resolution. Nature 612, 732-738 (2022). https://doi.org: 10.1038/s41586-022-05526-y
27 Garriock, R. J. et al. Lineage tracing of neuromesodermal progenitors reveals novel Wnt-dependent roles in trunk progenitor cell maintenance and differentiation. Development 142, 1628-1638 (2015). https://doi.org: 10.1242/dev.111922
28 Tzouanacou, E., Wegener, A., Wymeersch, F. J., Wilson, V. & Nicolas, J. F. Redefining the progression of lineage segregations during mammalian embryogenesis by clonal analysis. Dev Cell 17, 365-376 (2009). https://doi.org: 10.1016/j.devcel.2009.08.002
29 Schiebinger, G. et al. Optimal-Transport Analysis of Single-Cell Gene Expression Identifies Developmental Trajectories in Reprogramming. Cell 176, 928-943.e922 (2019). https://doi.org: 10.1016/j.cell.2019.01.006
30 Irie, N. et al. SOX17 is a critical specifier of human primordial germ cell fate. Cell 160, 253-268 (2015). https://doi.org: 10.1016/j.cell.2014.12.013 31 Sasaki, K. et al. Robust In Vitro Induction of Human Germ Cell Fate from Pluripotent Stem Cells. Cell Stem Cell 17, 178-194 (2015). https://doi.org: 10.1016/j.stem.2015.06.014
32 Sasaki, K. et al. The Germ Cell Fate of Cynomolgus Monkeys Is Specified in the Nascent Amnion. Dev Cell 39, 169-185 (2016). https://doi.org: 10.1016/j.devcel.2016.09.007
33 Zhu, Q. et al. Specification and epigenomic resetting of the pig germline exhibit conservation with the human lineage. Cell Rep 34, 108735 (2021). https://doi.org: 10.1016/j.celrep.2021.108735
34 Kobayashi, T. et al. Tracing the emergence of primordial germ cells from bilaminar disc rabbit embryos and pluripotent stem cells. Cell Rep 37, 109812 (2021). https://doi.org: 10.1016/j.celrep.2021.109812
35 Minn, K. T. et al. High-resolution transcriptional and morphogenetic profiling of cells from micropatterned human embryonic stem cell gastruloid cultures. bioRxiv, 2020.2001.2022.915777 (2020). https://doi.org: 10.1101/2020.01.22.915777
36 Kee, K., Gonsalves, J. M., Clark, A. T. & Pera, R. A. Bone morphogenetic proteins induce germ cell differentiation from human embryonic stem cells. Stem Cells Dev 15, 831-837 (2006). https://doi.org: 10.1089/scd.2006.15.831
37 Kee, K., Angeles, V. T., Flores, M., Nguyen, H. N. & Reijo Pera, R. A. Human DAZL, DAZ and BOULE genes modulate primordial germ-cell and haploid gamete formation. Nature 462, 222-225 (2009). https://doi.org: 10.1038/nature08562
38 Sugawa, F. et al. Human primordial germ cell commitment in vitro associates with a unique PRDM14 expression profile. Embo j 34, 1009-1024 (2015). https://doi.org: 10.15252/embi.201488049
39 Clark, A. T. et al. Spontaneous differentiation of germ cells from human embryonic stem cells in vitro. Hum Mol Genet 13, 727-739 (2004). https://doi.org: 10.1093/hmg/ddh088
40 Chen, D. et al. Germline competency of human embryonic stem cells depends on eomesodermin. Biol Reprod 97, 850-861 (2017). https://doi.org: 10.1093/biolre/iox138
41 Chen, D. et al. Human Primordial Germ Cells Are Specified from Lineage-Primed Progenitors. Cell Rep 29, 4568-4582.e4565 (2019). https://doi.org: 10.1016/j.celrep.2019.11.083
42 Hayashi, K., Ohta, H., Kurimoto, K., Aramaki, S. & Saitou, M. Reconstitution of the mouse germ cell specification pathway in culture by pluripotent stem cells. Cell 146, 519-532 (2011). https://doi.org: 10.1016/j.cell.2011.06.052
43 Falin, L. I. The development of genital glands and the origin of germ cells in human embryogenesis.
44 Yang, R. et al. Amnion signals are essential for mesoderm formation in primates. Nature Communications 12, 5126 (2021). https://doi.org: 10.1038/s41467-021-25186-2
45 Lawson, K. A. et al. Bmp4 is required for the generation of primordial germ cells in the mouse embryo. Genes Dev 13, 424-436 (1999). https://doi.org: 10.1101/gad.13.4.424
46 Aguilera-Castrejon, A. A.-O. et al. Ex utero mouse embryogenesis from pre-gastrulation to late organogenesis.
47 Inman, G. J. et al. SB-431542 is a potent and specific inhibitor of transforming growth factor-beta superfamily type I activin receptor-like kinase (ALK) receptors ALK4, ALK5, and ALK7. Mol Pharmacol 62, 65-74 (2002). https://doi.org: 10.1124/mol.62.1.65
48 Okae, H. et al. Derivation of Human Trophoblast Stem Cells. Cell Stem Cell 22, 50-63.e56 (2018). https://doi.org: 10.1016/j.stem.2017.11.004
49 Wang, C. et al. TGFβ inhibition enhances the generation of hematopoietic progenitors from human ES cell-derived hemogenic endothelial cells using a stepwise strategy. Cell Res 22, 194-207 (2012). https://doi.org: 10.1038/cr.2011.138
50 Bulatovic, I., Månsson-Broberg, A., Sylven, C. & Grinnemo, K. H. Human fetal cardiac progenitors: The role of stem cells and progenitors in the fetal and adult heart. Best Pract Res Clin Obstet Gynaecol 31, 58-68 (2016). https://doi.org: 10.1016/j.bpobgyn.2015.08.008
51 Buckingham, M., Meilhac, S. & Zaffran, S. Building the mammalian heart from two sources of myocardial cells. Nat Rev Genet 6, 826-835 (2005). https://doi.org: 10.1038/nrg1710
52 Pijuan-Sala, B. et al. A single-cell molecular map of mouse gastrulation and early organogenesis. Nature 566, 490-495 (2019). https://doi.org: 10.1038/s41586-019-0933-9
53 Chen, M. J., Yokomizo T Fau-Zeigler, B. M., Zeigler Bm Fau-Dzierzak, E., Dzierzak E Fau-Speck, N. A. & Speck, N. A. Runx1 is required for the endothelial to haematopoietic cell transition but not thereafter.
54 Boisset, J. C. et al. In vivo imaging of haematopoietic cells emerging from the mouse aortic endothelium. Nature 464, 116-120 (2010). https://doi.org: 10.1038/nature08764
55 Buhring, H. J. et al. The adhesion molecule E-cadherin and a surface antigen recognized by the antibody 9C4 are selectively expressed on erythroid cells of defined maturational stages.
56 Lammers, R. et al. Monoclonal antibody 9C4 recognizes epithelial cellular adhesion molecule, a cell surface antigen expressed in early steps of erythropoiesis.
57 Schindelin, J. et al. Fiji: an open-source platform for biological-image analysis. Nat Methods 9, 676-682 (2012). https://doi.org: 10.1038/nmeth.2019
58 Young, M. D. & Behjati, S. SoupX removes ambient RNA contamination from droplet-based single-cell RNA sequencing data. GigaScience 9, giaa151 (2020). https://doi.org: 10.1093/gigascience/giaa151
59 Hao, Y. et al. Integrated analysis of multimodal single-cell data. Cell 184, 3573-3587.e3529 (2021). https://doi.org: 10.1016/j.cell.2021.04.048
60 Lubatti, G. et al. CIARA: a cluster-independent algorithm for the identification of markers of rare cell types from single-cell RNA seq data. bioRxiv, 2022.2008.2001.501965 (2022). https://doi.org: 10.1101/2022.08.01.501965
61 Hie, B., Bryson, B. & Berger, B. Efficient integration of heterogeneous single-cell transcriptomes using Scanorama. Nat Biotechnol 37, 685-691 (2019). https://doi.org: 10.1038/s41587-019-0113-3
62 Wolf, F. A., Angerer, P. & Theis, F. J. SCANPY: large-scale single-cell gene expression data analysis. Genome Biol 19, 15 (2018). https://doi.org: 10.1186/s13059-017-1382-0
63 Schiebinger, G. et al. Optimal-Transport Analysis of Single-Cell Gene Expression Identifies Developmental Trajectories in Reprogramming.
64 Aguilera-Castrejon, A. et al. Ex utero mouse embryogenesis from pre-gastrulation to late organogenesis. Nature 593, 119-124 (2021). https://doi.org: 10.1038/s41586-021-03416-3
65 Bedzhov, I., Leung, C. Y., Bialecka, M. & Zernicka-Goetz, M. In vitro culture of mouse blastocysts beyond the implantation stages. Nature Protocols 9, 2732-2739 (2014). https://doi.org: 10.1038/nprot.2014.186

### Example 2

### Introduction

The classical Carnegie collection of human embryology from the 19^{th} and 20^{th} centuries^{1,2} has provided an essential foundation for the landmarks of human development. However, they do not provide details or mechanistic insights into critical events of very early development and disorders. Recently, in vitro models described as blastoids³⁻⁶, embryoids^{7,8} and gastruloids⁹⁻¹³ were developed to mimic pre- and post-gastrulation embryonic development. Bioengineering approaches have also resulted in micropatterned embryos^{14,15} and the use of microfluidics for developing posterior epiblast and amniogenesis^{16,17}. More recently, human pluripotent stem cell (hPSC)-based models of early post-implantation development captured the initial 14 days of embryo development¹⁸⁻²². These models have strengths and weaknesses, biases toward particular lineages, and both technical and regulatory barriers that prevent cultures for prolonged periods.

Successful generation of hematopoietic stem cells (HSCs) from pluripotent stem cells (PSCs) or endothelial cells (ECs) have been achieved by ectopic expression of key transcription factors^{23,24} and activation or inhibition of signalling pathways only in embryoid body models²⁴⁻²⁹. Most of these methods involve blood colony formation through co-culture of putative stem cell-derived HSCs with feeder cells, such as murine bone marrow-derived OP9 cells^{30,31} or addition of cocktail of cytokines to achieve haematopoiesis. Recently, integrated stem cell-based embryo models (SCBEM) suggested the emergence of early hematopoietic progenitors^{18,32}. However, they represented a primitive or pro-definitive hematopoietic wave as would be expected from pre-gastrulating D14 human embryos arising from extra-embryonic yolk sac. Similarly, non-integrated SCBEM, such as gastruloids¹¹, somitoids^{33,34} axioloids³⁵ and extended multilineage organised (EMLO) models¹³ have shown advancement with different aspects of early human development, however, are unable to give rise to hepatocytes, cardiomyocytes and hematopoietic cells thus far. Derivation of definitive, epiblast-derived HSCs capable of producing adult immune cells is considered a primary objective in the field³⁶. Despite major efforts, recapitulating this process in vitro has been hampered by technical challenges. For instance, work in animal models such as zebrafish suggests that conversion of PSCs into HSCs occurs at least in part through endothelial-to-haematopoietic transition (EHT)³⁷, yet this process has only been induced through the addition of cytokines and the use of feeder cells in vitro^{24,28,29,38}.

Here, we present a non-integrated SCBEM for generating a multilineage organogenesis system from human embryonic stem cells (hESCs) encompassing haematopoiesis by combining static and kinetic cultures³⁹. These aggregates self-organise into a three-dimensional multilineage organogenesis model in kinetic culture, which we refer to as non-integrated stem cell-based embryo model (or niSTEMBRYOs). The model depicts early human development recapitulating the formation of three germ layer derivatives: mesoderm, endoderm and ectoderm. Notably, these niSTEMBRYOs advance significantly, as judged by these models' transcriptome profiles, which suggests development equivalent to CS12-CS16 human embryos⁴⁰. The Carnegie stage (CS) 15 human embryonic tissues illustrate comparable expression of hematopoietic cells and their progenitors as observed in the niSTEMBRYOS. Here, we present a model in which the onset of organogenesis, including hepatocytes, contractile cardiomyocytes and haematopoietic cells together with endothelial and erythro-myeloid cells occur in parallel, involving transition from hematopoietic stem and progenitors to hematopoietic cells⁴¹.

### Results

Non-Integrated Stem Cell-Based Embryo Models (niSTEMBRYOs) Reveal Multilineage Organogenesis

We have previously shown that transient induction of human pluripotent stem cells (hPSCs) in the presence of Nodal agonist ACTIVIN-A (ACT) and WNT agonist CHIRON (CH) upregulates the expression of pluripotency and primitive streak (PS) markers⁴². Here, we used hESCs to induce cellular heterogeneity by culturing them for 24 h in presence of CH and ACT (FIG. 18A). The transiently induced cells expressed BRA (or TBXT), EOMES and SOX17 along with reduced expression of pluripotency markers, POU5F1 (or OCT4), NANOG and SOX2 (FIG. 19A).

To track the development and progression of the three-dimensional (3-D) aggregates, we explored methods to extend development by combining static with kinetic culture using a rotary system^{8,39,43} (FIG. 19B). First, the aggregates were cultured for four days in static system (Aggrewell™ plates) (FIG. 18A), resulting in the elongation of ~61% structures (2365/3850; N=5 experiments), exhibiting three germ layer derivatives (FIG. 18B-C; FIG. 19C) and amnion like cells (AMLC) (FIG. 18C). Having captured multilineage complex 3-D structures, our next aim was to progress these 3D aggregates to capture initiation of post-implantation organogenesis. Prolonged maturation is hindered by formation of necrotic cores at the centre of aggregates, and eventual regression of these structures. Therefore, we decided to use roller culture system, which has improved the developmental potential of mouse embryos⁴⁴ and mouse stem cell-based embryo models (SCBEM)⁷, that could also offer an avenue for extended development of 3-D aggregates in our model.

Visibly elongated aggregates (FIG. 18C; FIG. 19C) were selected on D4 (N=1170, n=5 experiments) for transfer to a hypoxic (5% O2, 5% CO2) rotary system³⁹ using the same media as for static culture (FIG. 18A; FIG. 19B). However, the aggregates adapted poorly and failed to grow or display consistent expression of protein reporters (FIG. 19E). We attempted other approaches, including the supplementation with human cord serum (HCS)⁸ and culture in a solidified extra-cellular matrix (Collagen type I) in the static cultures before transferring and adding a soluble laminin-rich matrix in the rotary cultures (10% Matrigel®). The addition of Collagen to static cultures caused webbing of the structure into poorly organised aggregates that were no longer suited for transfer to the kinetic cultures (FIG. 19F). Similarly, supplementation with HCS resulted in irregular structures without discernible or reproducible patterns in the kinetic culture (FIG. 19G-H). As the use of SB431542 (hereafter called SB), a small molecule inhibitor of the TGF-ß pathway⁴⁵, promotes hemogenic endothelium²⁷ and increases development of endodermal and ectodermal lineage⁴⁶, we tested its effect in our system together with WNT agonist, Chiron, that promotes mesoderm lineage⁴⁷, to induce all three germ layers. We found that the addition of SB had a striking effect on development in the kinetic culture (FIG. 18D), with or without supplementation with 10% soluble Matrigel (FIG. 19). Unlike other conditions, where these aggregates regressed and became necrotic, the aggregates cultured with SB adapted to kinetic culture, successfully doubling in size after four days and quadrupling after 14 days (FIG. 18D). We termed these structures non-integrated stem cell-based embryo models (niSTEMBRYOs), and continued investigation of organogenesis within these kinetic-compatible culture conditions.

After 7 days in kinetic culture with SB, we observed periodic and highly localised contractions of cells in ~10% of niSTEMBRYOs, which increased to ~72% by D8 (FIG. 18E). While random contractions of EBs have been previously observed⁴⁸, it was notable that these periodic contractions were not evident in niSTEMBRYOs cultured with HCS or with CH alone (FIG. 18E). To assess whether contractions were due to the development of contractile cardiomyocytes (CMS), we performed wholemount immunofluorescence (IF) staining and observed the expression of NKX2-5 and Myosin heavy chain (MYH) 2, key regulators of cardiac morphogenesis and skeletal muscle contraction (FIG. 18F). We next sought to extend kinetic cultures of niSTEMBRYOs and found that from D14 onwards ~16% of them (N=196/1170, 5 experiments) began to develop visible red hemogenic zones (FIG. 18G-H). Similar to the observation of contractile cardiomyocytes, the appearance of red hemogenic zones depended on SB supplementation to rotary cultures, and this phenotype did not appear when the aggregates were cultured in the presence of CH or HCS alone (FIG. 18H).

The observation of a striking ‘red’ hemogenic phenotype suggested the possibility of the initiation of embryonic haematopoiesis in the niSTEMBRYOs. To establish an explicit link between visible red inclusions and a hemogenic phenotype, we split niSTEMBRYOs into two groups; with (RED) or without (clear or control) putative hemogenic zones (FIG. S2A) and dissociated them for quantitative flow cytometric analysis. The ‘red’ niSTEMBRYOs contained approximately 4-5% hematopoietic cells (HCs), with significantly enriched CD45⁺ live cells (FIG. 18I-J). Remarkably, the hematopoietic cells in niSTEMBRYOs exhibited signs of endothelial-to-hematopoietic transition, as suggested by the presence of CD31⁺ putative endothelial cells, CD45⁺ hematopoietic cells, and a gradient of expression including a CD45⁺CD31⁺ intermediary cells (FIG. 1K8). Furthermore, CD31⁺ cells expressed higher levels of hematopoietic stem and progenitor cell (HSPC) markers such as CD34 and c-KIT. However, the loss of both markers (FIG. 20B-C) suggests that CD45⁺ population might be undergoing further hematopoietic progression. These results suggest that all factors required for hematopoiesis are being provided by the native environment within the niSTEMBRYOS.

Transcriptome Characterization of niSTEMBRYOs Confirms Multilineage Organogenesis

To confirm the occurrence of multilineage organogenesis and to gain insights into the human hematopoietic development, we investigated the transcriptome of niSTEMBRYOs by 10× single cell RNA sequencing (scRNAseq), sampled at D8 and D14. We performed unsupervised clustering and identified 8 major cell types from two developmental time points (FIG. 21A; FIG. 20D-E). While cell types such as amnion (VTCN1⁺, WNT6⁺, TFAP2A⁺), mesenchyme (PCOLCE⁺, COL1A2⁺, HAND1⁺), cardiomyocytes (ACTC1⁺, NKX2.5⁺, TNNT2⁺), hepatocytes (AFP⁺, ALB⁺, CEBPA⁺) and blood progenitors 1 (PECAM1/CD31⁺, CD34⁺, CDH5⁺) were common to both developmental time points, cell types such as splanchnic mesoderm (WT1⁺, LHX2⁺, GATA4⁺), erythroids (GATA1⁺, KLF1⁺, HBA1⁺) and blood progenitors 2 (RUNX1⁺, PTPRC/CD45⁺, CD44⁺) were present only at D14 (FIG. 21B, FIG. 20F-G).

To assess what developmental stage the niSTEMBRYOs corresponded to, we next integrated our dataset with the transcriptome of 4-6 week-old human embryos, corresponding to Carnegie stages (CS) 12-16⁴⁰ (FIG. 22A). Heatmap projection of D14 niSTEMBRYO clusters displayed their resemblance to the transcriptome and cell type composition of post-gastrulation development obtained from CS12-CS16 embryos (FIG. 21C; FIG. 22A). Notably, splanchnic mesoderm, cardiomyocytes and hepatocytes from niSTEMBRYOs aligned with splanchnic lateral plate mesoderm (LPM) and hepatocytes from CS12-CS16 human embryos, and erythroid and blood progenitor (BP)-2 from D14 aligned with blood, whereas blood progenitor (BP)-1 from D14 aligned with endothelium. Thus, we labelled BP-2 as hematopoietic cells (HCs) and BP-1 as endothelial cells (ECs) for further analysis. Although some but not all cell types from in vivo embryos were detected in niSTEMBRYOs, the correlation with some of the CS12-CS16 human embryos cell types confirms the advanced development of niSTEMBRYOs.

Next, we compared the transcriptome of cardiomyocytes (CMs) detected in niSTEMBRYOs with that of human embryonic heart cells obtained from CS12-CS16 human embryos⁴⁰. The cardiomyocytes obtained from niSTEMBRYOs aligned to atrial and ventricular cardiomyocytes (FIG. 22B). The niSTEMBRYO-cardiomyocytes primarily exhibited features of cardiac morphogenesis with enriched expression of ACTC1, TNNT2, MYH6 (FIG. 21B, FIG. 20F-G). Similarly, ventricular cardiomyocytes expressing MYL7 and IRX3 were enriched in niSTEMBRYO-cardiomyocytes (FIG. 20F-G). The presence of cardiac-specific markers supports the typical features of early cardiomyocytes in niSTEMBRYOs as reported recently in human cardioids model⁴⁹.

To further investigate the identity of putative hematopoietic cells present in D14 niSTEMBRYOs, we isolated and recombined blood progenitor clusters from D8 and D14 as well as erythroids from D14 scRNAseq data for in depth analysis (FIG. 21D). As anticipated, this approach confirmed the expression of endothelial markers such as CD34 and CD31 (or PECAM1), whereas expression of hematopoietic markers such CD45 (or PTPRC), MYB, ITGA2B, SPN, GFI1B, SPINK2 and CD44 were detected in HCs only (FIG. 2E, FIG. S3C). Both, embryonic haemoglobin markers, HBE1 and HBG1 and adult haemoglobin markers, HBA1 and HBA2 were detected in erythroids (FIG. 21E; FIG. 22C). Furthermore, these erythroids also expressed HBE2, HBG2, KLF1 and GATA1, markers detected during early embryonic hematopoietic and erythropoietic development (FIG. 22C). These results confirm that niSTEMBRYOs exhibit transcriptomic features of endothelial cells, hematopoietic cells and erythroids that are observed during the origin of haematopoiesis.

To look into the subtypes of blood cells detected in niSTEMBRYOs, we isolated hematopoietic cells (HCs) and erythroids from D14 and compared them with blood cells from CS12-CS16 human embryo dataset⁴⁰. We observed that two erythroids clusters from D14 niSTEMBRYOs were similar to in vivo erythroids from human embryos (FIG. 21F; FIG. 22A), whereas HCs from D14 niSTEMBRYOs mapped to different types of blood cells, including macrophages, megakaryocytes, eosinophil, basophil, mast cell progenitor, lymphocytes and hematopoietic stem and progenitor cells (HSPC) from CS12-CS16 human embryos (FIG. 21; FIG. 22A). Further analysis of HCs revealed the detection of key HSPC markers, including KIT, RUNX1, GATA3, SOX7 and CD34, among others (FIG. 21H). Remarkably, markers of megakaryocytes, such as GP1BA, GP1BB and NFE2, as well as markers of macrophages, such as CD68, CD86, MRC1 and TREM2, were enriched in D14 niSTEMBRYOs. Furthermore, detection of eosinophil, basophil and mast cell progenitors (GATA2, IL1B, MYC, STAT5A and SOX4) as well as lymphocytes (CD52, CD69, HOPX, SPINK2, IL7R and NKG7) confirmed the induction of haematopoiesis in niSTEMBRYOs (FIG. 21G-H; FIG. 22A). These results suggest that niSTEMBRYOs display the features of haematopoiesis and erythropoiesis that resemble human embryonic blood obtained from CS12-CS16 developmental stages.

Inhibition of TGF-b Signalling During Early niSTEMBRYO Development Represses Haematopoiesis

Induction of mesoderm lineage and inhibition of TGF-b signalling pathway are crucial steps for generating blood organoids^{24,27}. Inhibition of TGF-b signalling after mesoderm induction promotes the generation of hematopoietic and endothelial progenitors (HEPs) in mice, expressing CD34, CD31 and VE-cadherin (VEC), whereas inhibition of TGF-b signalling before mesoderm induction downregulates the expression of mesodermal markers and reduces the number of hemogenic endothelial progenitors⁴⁶. We showed above that addition of TGF-b inhibitor, SB43, from day 4 onwards not only promoted adaptation to kinetic cultures, but also promoted haematopoiesis in niSTEMBRYOs (now termed as niSTEMBRYO:SB^{late}) (FIG. 18G-K, 2E-H). To investigate the effect of TGF-b inhibition on hematopoietic development before or during mesoderm induction, we cultured niSTEMBRYOs in presence of SB from D1 (now termed as niSTEMBRYO:SB^{early}) (FIG. 23A). While the primary germ layer markers, including SOX17, SOX2 and TBXT and amnion marker, TFAP2A, were not affected until D2 (FIG. 24A; FIG. 23B), expression of mesodermal marker, TBXT, was downregulated by D4 (FIG. 24B). Interestingly, primitive gut was observed in this condition as shown by the formation of tubular structure, expressing SOX17 and SOX2 with some overlap between the two markers, suggesting an appearance of hindgut (FIG. 23C). Of note, hindgut was absent in the niSTEMBRYOs cultured with SB from D4 (niSTEMBRYO:SB^{late}). A major difference was that no red hemogenic phenotype was observed in the niSTEMBRYOs cultured in presence of SB from D1 (niSTEMBRYO:SB^{early}) (FIG. 23D). However, appearance of contracting cardiomyocytes was not affected, with no significant difference in contractile niSTEMBRYOs or TNNT2 expression observed between the two conditions (niSTEMBRYO:SB^{early} and niSTEMBRYO:SB^{late}) (FIG. 23E-G).

To characterize the transcriptome profile of niSTEMBRYO:SB^{early} and compare them with niSTEMBRYO:SB^{late}, we performed scRNAseq at D8 and D14 and identified 14 major cell types by unsupervised clustering (FIG. 24; FIG. 23H-I). Amnion (TFAP2A⁺, GABRP⁺, VTCN1⁺), neural crest (FOXD3⁺, S100B⁺, HOXB9⁺), cardiomyocytes (TNNT2⁺, TNNI1⁺, ACTC1⁺), hindgut (CDX2⁺, SHH⁺, EPCAM⁺), mesenchyme (PCOLCE⁺, COL1A2⁺, COL6A3⁺), neural precursors (SOX2⁺, ZIC5⁺, POU3F2⁺), paraxial/presomitic mesoderm (MEOX1⁺, SIX4⁺, FOXC2⁺) and blood progenitors (CD34⁺, CD31⁺, CDH5⁺) were detected in niSTEMBRYO:SB^{early} at D8 and D14, whereas neuronal progenitors (ISI1⁺, ZEB2⁺, FOXC1⁺), hepatocytes (AFP⁺, ALB⁺, TF⁺) and musculoskeletal precursors (NEFM⁺, SIX1⁺, TNNT1⁺) were detected in niSTEMBRYO:SB^{early} at D14 only (FIG. 24; FIG. 25A-B).

Next, we compared the transcriptional profile of niSTEMBRYO:SB^{early} with in vivo developed embryos by integrating our dataset with 4-6 weeks human embryos at Carnegie stages (CS) 12-1640 (FIG. 25C-D). Heatmap projection confirmed the identity of D14 SB^{early} clusters and validated their identities to the transcriptome and cell type composition of post-gastrulation human development obtained from CS12-CS16 embryos ((FIG. S5E). These results suggest that similar to SB^{late}, SB^{early} cells resemble the post-implantation human embryo lineages. We then investigated the difference between SB^{early} and SB^{late} cells by comparing their transcriptional profiles at D8 (FIG. 26A) and D14 (FIG. 26B). niSTEMBRYO:SB^{early} displayed the formation of hindgut, expressing SOX2 and SOX17 (FIG. 23C-F, 26A-B) which was absent in SB^{late} samples (FIG. 26A-B). Furthermore, we detected paraxial mesoderm, presomitic mesoderm and neuronal lineages in SB^{early} condition that were absent in SB^{late} condition. However, D14 niSTEMBRYO:SB^{early} lacked splanchnic mesoderm cells that can give rise to hematopoietic lineage^{50,51}, which was detected in SB^{late} group (FIG. 26B).

Transcriptome profiles of D8 and D14 niSTEMBRYOs cultured with SB43 from D1 (SB^{early}) revealed the detection of endothelial cells expressing CDH5, CD34, CD31 (or PECAM1), KDR (or FLK1), SOX7 and THY1 (FIG. 24C-D; FIG. 26A-B). However, they lacked hematopoietic cells as observed in D14 SB^{late} samples (FIG. 21D-E). Interestingly, endothelial cells detected in D8 and D14 SB^{early} niSTEMBRYOs were transcriptionally similar to endothelial cells detected in D8 and D14 SB^{late} niSTEMBRYOs. However, these endothelial cells were transcriptionally different from blood hematopoietic cells detected in D14 SB^{late} niSTEMBRYOs (FIG. 21B-E, 3D; FIG. 25E). When integrated with CS12-CS16 human embryo dataset, we observed that endothelial cells from both groups resembled the endothelium, whereas blood cells from in vivo human embryos correlated with D14 SB^{late} hematopoietic cells and erythroids (FIG. 21C, FIG. 25E). Differential gene expression (DGE) analysis between the endothelial cells (ECs) of D8 niSTEMBRYOs revealed the enrichment of markers, including ITGB1, CXCR4, NRARP, GJA4, IGFBP4 in SB4^{late} niSTEMBRYOs (FIG. 24E) that are linked to gene ontology terms associated with angiogenesis, endothelium development and maintenance of blood-brain barrier (FIG. 26C). These results suggest that only blood progenitor cells from niSTEMBRYO:SB^{late} (hematopoietic cells) that are correlated to in vivo human embryos (blood cells) give rise to blood, but not the ones from niSTEMBRYO:SB^{early} (endothelial cells).

A vascular endothelial growth factor receptor-1 (VEGF1 or FLT1) that is expressed on vascular endothelial cells and is also found in peripheral blood monocytes⁵², was enriched in D8 niSTEMBRYO:SB^{late} compared to D8 niSTEMBRYO:SB^{early} (FIG. 24E; FIG. 26D). Similarly, a receptor of the TGF-b superfamily, Endoglin (ENG), that is required for hematopoietic cell fate development during embryogenesis⁵³ was enriched in niSTEMBRYO:SB^{late} compared to D8 niSTEMBRYO:SB^{early} (FIG. 24E; FIG. 26D). Furthermore, differential gene expression analysis of endothelial cell clusters at D14 revealed the enrichment of TMEM88, TPM1, PRTG, HOXB7 and MEIS2 in niSTEMBRYO:SB^{late} that were associated with branching in blood vessel morphogenesis and regulation of actin cytoskeleton organization (FIG. 24F; FIG. 26E-F). Similarly, endothelial cells of D14 niSTEMBRYO:SB^{early} were enriched for markers, including RNF19A, CAV1, REN and BNIP3, which are associated with cartilage morphogenesis (FIG. 24F; FIG. 26E-F). Notably, two secreted ligands, Stanniocalcin 1 (STC1) and Fibroblast growth factor 23 (FGF23), stand out in particular (FIG. 24G), as STC1 reportedly inhibits hematopoietic stem cell proliferation in adult leukaemia⁵⁴, and FGF23 inhibits pre- and postnatal erythropoeisis⁵⁵. These results suggest that inhibition of TGF-b signalling pathway from earlier time point (referred to as niSTEMBRYO:SB^{early}) biases development towards ectodermal lineage and away from splanchnic mesoderm fate, which may result in a paracrine signalling environment that leads to inhibition of haematopoiesis (FIG. 24H; FIG. 26G).

niSTEMBRYOs Display the Transition from Hematopoietic Stem and Progenitors to Hematopoietic Cells

To investigate whether hematopoietic cells observed in the niSTEMBRYOs exhibited the transition from hematopoietic stem and progenitor cells (HSPCs), we regrouped them into four subclusters (0, 1, 2 and 3) (FIG. 27A). Pseudotime trajectory analysis of the subclustered groups revealed the bifurcation of hematopoietic stem and progenitor cells (HSPCs) into two branches, A and B (FIG. 28A, FIG. 27B). HSPC markers such as RUNX1, PECAM1 (or CD31) and CD34 were upregulated in the progenitor cells, whereas hematopoietic markers such as PTPRC (or CD45) and GFI1B were downregulated in this group (FIG. 27B). Eventually, markers of erythro-myeloid cells (CD4, CD36, CD9, GATA1 and MPA) were upregulated in the hematopoietic lineages in branch A and B (FIG. 27B-C). A recent study on human embryos has reported the molecular identity of hematopoietic stem cells (HSC), expressing RUNX1, HOXA9, HLF, MECOM, MLLT3 and SPINK228. Detection of these signature markers in niSTEMBRYOs confirms the presence of hematopoietic stem cells (FIG. 28B; FIG. 27D). Similarly, HSC regulator and aorta-gonad-mesonephros (AGM) hematopoietic stem cell markers, SPINK2 and RAB27B, were enriched in niSTEMBRYOs, thus confirming the molecular identity of HSCs (FIG. 4B; FIG. S7D). Similar to in vivo human embryos, hemogenic endothelial and HSC markers, MYCN and KCNK17, were detected in the hematopoietic stem and progenitor cells as well as in the hematopoietic cells in the niSTEMBRYOs, indicating the transition from hemogenic progenitors to hematopoietic lineage (FIG. 28B; FIG. 27D).

Early hematopoietic cell markers, LIN28A and STXBP6, definitive hematopoietic stem cell markers, KIT and ITGA4, as well as definitive transcript regulators, EAF2, BCL11A and MLLT3 were detected in the niSTEMBRYOs, as in human embryos²⁸, thus suggesting a wave of transition from hemogenic endothelial to hematopoietic cells (FIG. 28B; FIG. 27E). Notably, markers of erythro-myeloid progenitors (EMP), including MRC1 and CX3CR1, were detected in hematopoietic cell lineages (FIG. 28B, FIG. 27C, 27E). Similarly, markers of erythro-myeloid progenitors (EMP), GATA1, ITGA2B, GFI1B, MYCT1, FCGR3A (or CD16) and FUT4 (or CD15) were enriched in hematopoietic cell lineages (FIG. 28B, FIG. 27C, 27E). While immune cell markers, such as CD4 and CD36 were downregulated in the progenitor cells, these markers were upregulated in the hematopoietic lineage (branch A) (FIG. 28B; FIG. 27C). Hematopoietic cells also expressed the markers of immune cells and myeloid cells, including CD9, FABP3, GAS6, MRC1 and SLC15A3 (FIG. 28B; FIG. 27BC). These results confirm that hematopoietic stem and progenitor cells transition to hematopoietic lineage, a critical feature occurring during embryonic haematopoiesis.

Notably, transcription factor, RUNX1, and its downstream target, MYB, was detected in D14 niSTEMBRYOs cultured with SB from D4 only (SB^{late}) and was absent in D14 niSTEMBRYOs cultured with SB from D1 (SB^{early}) (FIG. 28C; FIG. 29A). Consequently, the hematopoietic marker CD45 (or PTPRC) was detected in niSTEMBRYO:SB^{late} only (FIG. 28B-C). Although endothelial markers, such as PECAM1 (or CD31) and CD34 were detected in both groups (niSTEMBRYO:SB^{late} and niSTEMBRYO:SB^{early}) (FIG. 28B; FIG. 29A), hematopoietic stem cell regulators, RUNX1, SPINK2, MYB, BCL11A and STXBP6 as well as mature hematopoietic marker, such as CD45 and GATA1, was only detected in niSTEMBRYO:SB^{late} group (FIG. 28B-C). These results suggest that hematopoietic stem and progenitor cells are present only in D14 niSTEMBRYO:SB^{late} that give rise hematopoietic cells^{28,40}. Here, we observed that inhibition of TGF-b signalling from D1 resulted in the downregulation of TBXT (FIG. 3C) with eventual loss of HSC regulators (FIG. 28B), thus repressing haematopoiesis in niSTEMBRYO:SB^{early}. These results suggest that the transition from hematopoietic stem and progenitors to hematopoietic cells in SB^{late}, but not in SB^{early} niSTEMBRYOs, is facilitating induction of haematopoiesis and erythropoiesis in vitro.

Accordingly, we performed wholemount IF staining to characterize D14 niSTEMBRYOs, and detected discernible regions expressing alpha-fetoprotein (AFP) and Albumin (ALB), confirming the presence of foetal liver progenitors (or hepatocytes) in niSTEMBRYOs (FIG. 4D, FIG. S8B-C). CD45⁺ hematopoietic cells were also observed (FIG. 28D), suggesting that niSTEMBRYO structures may possess a suitable environment for acquiring hematopoietic development. Key hepatocyte markers, including alfa-fetoprotein (AFP), albumin (ALB), APOA1, APOB and TF were detected in the transcriptome profile of niSTEMBRYOs (FIG. 21B), suggesting the presence of putative progenitors of the foetal liver cells within these structures. Remarkably, cells expressing RUNX1⁺CD45, RUNX1⁺CD45⁺ and CD45⁺RUNX1⁻ were observed in D14 niSTEMBRYOs (FIG. 28E), coinciding with previously acquired flowcytometry results of single and double positive CD45⁺ and CD31⁺ populations (FIG. 18K).

Finally, to investigate the expression of hematopoietic and progenitor cells during definitive haematopoiesis in human post-implantation embryos, we performed IF staining on human embryonic tissues obtained from Carnegie stage (CS) 15 developmental stage (FIG. 28F; FIG. 29D). We observed CD45⁺ hematopoietic cells throughout the embryonic tissue, whereas RUNX1⁺ hematopoietic progenitor cells were mostly localised in the mesonephros (ventral) and spinal cord (dorsal) regions (FIG. 28F). A closer look at the mesonephros region revealed the expression of CD45⁺ hematopoietic cells, RUNX1⁺ hematopoietic progenitor cells and CD45⁺RUNX1⁺ intermediary cells, similar to what we observed in the niSTEMBRYOs (FIG. 28E). Another section of the CS15 human embryonic tissue revealed the accumulation of CD45⁺ hematopoietic cells in the mesonephros region adjacent to RUNX1⁺ hematopoietic progenitor cells (FIG. 29D). These results suggest that transition from hemogenic progenitors to hematopoietic cells occurs in niSTEMBRYOs as in human embryos during definitive haematopoiesis⁴¹.

### DISCUSSION

Here, we show that transiently induced hESCs self-organise into 3D multilineage representation of early human organogenesis, which display the emergence of primary germ layers that continue to develop significantly, and capture key aspects of early human development⁴⁰. We observed hematopoietic development in the context of early embryogenesis, starting from the self-organization of hESCs into three germ layer derivatives, which is different from previously described models of blood organoids obtained from directly differentiated pluripotent stem cells (PSCs) into embryoid bodies^{24,27-29,38} or integrated SCBEM^{18,32}. Recent human gastruloids model¹¹ has paved the way to generate non-integrated 3D SCBEM, leading the development of somitoids^{33,34}, axioloids³⁵ and extended multilineage organized (MLOs) gastruloids¹³. Emergence of hematopoietic stem and progenitors and their transition to hematopoietic cells in the niSTEMBRYOs, including erythroids, megakaryocytes, macrophages, eosinophil, basophil and mast cell progenitors as well as lymphocytes displays an intriguing observation in any SCBEMs thus far. Furthermore, the similarity of cell types detected at D14 niSTEMBRYOs with the previously described CS12-CS16 human embryos and eventual blood formation showcase the advancement of our model over previous in vitro systems. Notably, expression of hematopoietic progenitor marker, RUNX1, and hematopoietic marker, CD45, in human embryonic tissue was similar to that observed in niSTEMBRYOs, potentially recapitulating in vivo development.

Since there is an evident lack of extra-embryonic yolk sac and trophoblast cells in niSTEMBRYOs, hematopoietic cells in the model we describe here is likely of intra-embryonic in origin. Recently, a different route of hematopoietic development via posterior primitive streak has been reported in mouse embryos⁵⁶, suggesting a hematopoietic wave independent of extra-embryonic yolk-sac. Moreover, MYB and ANGPT1, regulators of definitive haematopoiesis^{41,57-59}, were detected in the hematopoietic stem and progenitor cells (HSPCs) in the niSTEMBRYOS (FIG. 21B; FIG. 27D, 29E). Taken together, detection of hematopoietic stem cell signature markers, RUNX1, MECOM, HOXA9, MLLT3, SPINK2 and HLF, followed by hematopoietic and erythro-myeloid markers, GATA1, HBE1, HBE2, MYCT1, ITGA2B, GFI1B, FUT4 (CD15) and FCGR3A (CD16) suggests an alignment with the embryonic origin^{28,40} of hematopoietic cells in niSTEMBRYOs (FIG. 27C, 27E, 29F). The loss of mesoderm marker, TBXT, and upregulation of the secreted ligands such as STC1 and FGF23 in the endothelial cells (ECs) of D14 niSTEMBRYO:SB^{early} might prevent the emergence of hematopoietic stem and progenitor cells in SB^{early} niSTEMBRYOs (FIG. 28B; FIG. 29G). However, how HSC regulators are controlled by inhibiting TGF-b signalling requires further investigation. Notably, the 3D niSTEMBRYOs model can capture tissue-scale events of post-implantation human development and interactions during haematopoiesis, suggesting that critical features such as immune-mediated modulation of development could be studied within niSTEMBRYOs⁶⁰, complementing a recent study on the foetal gut⁶¹. Our model also reveals that neuronal precursors (ectoderm), hindgut (endoderm) and presomitic mesoderm (mesoderm) cells can be induced in niSTEMBRYOs depending on the modulation of TGF-b signalling at different time points. This suggests that multilineage organogenesis with lineage-specific interests can be achieved using this model, thus offering the potential of niSTEMBRYO-derived cells for cell therapies and regenerative medicine.

### Example 2 References

1 Morgan, L. M. 1 online resource (329 p.) (University of California Press, Berkeley, 2009).
2 Streeter, G. L. Developmental horizons in human embryos; a review of the histogenesis of cartilage and bone. https://doi.org/D-CLML: 4917:194k1 OTO-NLM
3 Rivron, N. C. et al. Blastocyst-like structures generated solely from stem cells. Nature 557, 106-111 (2018). https://doi.org/10.1038/s41586-018-0051-0
4 Li, R. et al. Generation of Blastocyst-like Structures from Mouse Embryonic and Adult Cell Cultures. Cell 179, 687-702.e618 (2019). https://doi.org/10.1016/j.cell.2019.09.029
5 Kagawa, H. et al. Human blastoids model blastocyst development and implantation. Nature 601, 600-605 (2022). https://doi.org/10.1038/s41586-021-04267-8
6 Liu, X. A.-O. et al. Modelling human blastocysts by reprogramming fibroblasts into iBlastoids.
7 Amadei, G. et al. Embryo model completes gastrulation to neurulation and organogenesis. Nature 610, 143-153 (2022). https://doi.org/10.1038/s41586-022-05246-3
8 Tarazi, S. et al. Post-gastrulation synthetic embryos generated ex utero from mouse naive ESCs. Cell 185, 3290-3306.e3225 (2022). https://doi.org/10.1016/j.cell.2022.07.028
9 Beccari, L. et al. Multi-axial self-organization properties of mouse embryonic stem cells into gastruloids. Nature 562, 272-276 (2018). https://doi.org/10.1038/s41586-018-0578-0
10 van den Brink, S. C. et al. Symmetry breaking, germ layer specification and axial organisation in aggregates of mouse embryonic stem cells. Development 141, 4231-4242 (2014). https://doi.org/10.1242/dev.113001
11 Moris, N. et al. An in vitro model of early anteroposterior organization during human development. Nature 582, 410-415 (2020). https://doi.org/10.1038/s41586-020-2383-9
12 Simunovic, M. et al. A 3D model of a human epiblast reveals BMP4-driven symmetry breaking. Nat Cell Biol 21, 900-910 (2019). https://doi.org/10.1038/s41556-019-0349-7
13 Olmsted, Z. T. & Paluh, J. L. Co-development of central and peripheral neurons with trunk mesendoderm in human elongating multi-lineage organized gastruloids. Nature Communications 12, 3020 (2021). https://doi.org/10.1038/s41467-021-23294-7
14 Warmflash, A., Sorre, B., Etoc, F., Siggia, E. D. & Brivanlou, A. H. A method to recapitulate early embryonic spatial patterning in human embryonic stem cells. Nat Methods 11, 847-854 (2014). https://doi.org/10.1038/nmeth.3016
15 Minn, K. T. et al. High-resolution transcriptional and morphogenetic profiling of cells from micropatterned human ESC gastruloid cultures. Elife 9 (2020). https://doi.org/10.7554/eLife.59445
16 Zheng, Y. et al. Controlled modelling of human epiblast and amnion development using stem cells. Nature 573, 421-425 (2019). https://doi.org/10.1038/s41586-019-1535-2
17 Shao, Y. et al. A pluripotent stem cell-based model for post-implantation human amniotic sac development. Nat Commun 8, 208 (2017). https://doi.org/10.1038/s41467-017-00236-w
18 Oldak, B. et al. Complete human day 14 post-implantation embryo models from naive ES cells. Nature (2023). https://doi.org/10.1038/s41586-023-06604-5
19 Weatherbee, B. A. T. et al. Pluripotent stem cell-derived model of the post-implantation human embryo. Nature (2023). https://doi.org/10.1038/s41586-023-06368-y
20 Pedroza, M. et al. Self-patterning of human stem cells into post-implantation lineages. Nature (2023). https://doi.org/10.1038/s41586-023-06354-4
21 Joshua, H. et al. Modelling Human Post-Implantation Development via Extra-Embryonic Niche Engineering. bioRxiv, 2023.2006.2015.545118 (2023). https://doi.org/10.1101/2023.06.15.545118
22 Gege, Y. et al. Establishment of a novel non-integrated human pluripotent stem cell-based gastruloid model. bioRxiv, 2023.2006.2028.546720 (2023). https://doi.org/10.1101/2023.06.28.546720
23 Kissa, K. & Herbomel, P. Blood stem cells emerge from aortic endothelium by a novel type of cell transition.
24 Motazedian, A. et al. Multipotent RAG1+ progenitors emerge directly from haemogenic endothelium in human pluripotent stem cell-derived haematopoietic organoids. Nature Cell Biology 22, 60-73 (2020). https://doi.org/10.1038/s41556-019-0445-8
25 Lempereur, A. et al. The TGFβ pathway is a key player for the endothelial-to-hematopoietic transition in the embryonic aorta.
26 Vargel, Ô. et al. Activation of the TGFβ pathway impairs endothelial to haematopoietic transition.
Ng, E. S. et al. Differentiation of human embryonic stem cells to HOXA+ hemogenic vasculature that resembles the aorta-gonad-mesonephros. Nature Biotechnology 34, 1168-1179 (2016). https://doi.org/10.1038/nbt.3702
28 Calvanese, V. et al. Mapping human haematopoietic stem cells from haemogenic endothelium to birth. Nature 604, 534-540 (2022). https://doi.org/10.1038/s41586-022-04571-x
29 Piau, O. et al. Generation of transgene-free hematopoietic stem cells from human induced pluripotent stem cells.
30 Sturgeon, C. M., Ditadi, A., Awong, G., Kennedy, M. & Keller, G. Wnt signaling controls the specification of definitive and primitive hematopoiesis from human pluripotent stem cells.
31 Nakano, T., Kodama H Fau-Honjo, T. & Honjo, T. Generation of lymphohematopoietic cells from embryonic stem cells in culture.
32 Hislop, J. et al. Modeling post-implantation human development to yolk sac blood emergence. Nature (2023). https://doi.org/10.1038/s41586-023-06914-8
33 Sanaki-Matsumiya, M. A.-O. X. et al. Periodic formation of epithelial somites from human pluripotent stem cells.
34 Miao, Y. A.-O. et al. Reconstruction and deconstruction of human somitogenesis in vitro. LID-10.1038/s41586-022-05655-4 [doi].
35 Yamanaka, Y. et al. Reconstituting human somitogenesis in vitro. LID-10.1038/s41586-022-05649-2 [doi].
36 Ottersbach, K. A.-O. Endothelial-to-haematopoietic transition: an update on the process of making blood.
37 Chen, M. J., Yokomizo, T., Zeigler, B. M., Dzierzak, E. & Speck, N. A. Runx1 is required for the endothelial to haematopoietic cell transition but not thereafter. Nature 457, 887-891 (2009). https://doi.org/10.1038/nature07619
38 Ng, E. S., Davis Rp Fau-Azzola, L., Azzola L Fau-Stanley, E. G., Stanley Eg Fau-Elefanty, A. G. & Elefanty, A. G. Forced aggregation of defined numbers of human embryonic stem cells into embryoid bodies fosters robust, reproducible hematopoietic differentiation.
39 New Da Fau-Cockroft, D. L. & Cockroft, D. L. A rotating bottle culture method with continuous replacement of the gas phase.
40 Xu, Y. et al. A single-cell transcriptome atlas profiles early organogenesis in human embryos. Nature Cell Biology 25, 604-615 (2023). https://doi.org/10.1038/s41556-023-01108-w
41 Zeng, Y. et al. Tracing the first hematopoietic stem cell generation in human embryo by single-cell RNA sequencing. Cell Research 29, 881-894 (2019). https://doi.org/10.1038/s41422-019-0228-6
42 Kobayashi, T. et al. Principles of early human development and germ cell program from conserved model systems. Nature 546, 416-420 (2017). https://doi.org/10.1038/nature22812
43 Aguilera-Castrejon, A. A.-O. et al. Ex utero mouse embryogenesis from pre-gastrulation to late organogenesis.
44 Aguilera-Castrejon, A. et al. Ex utero mouse embryogenesis from pre-gastrulation to late organogenesis. Nature 593, 119-124 (2021). https://doi.org/10.1038/s41586-021-03416-3
45 Inman, G. J. et al. SB-431542 is a potent and specific inhibitor of transforming growth factor-beta superfamily type I activin receptor-like kinase (ALK) receptors ALK4, ALK5, and ALK7. Mol Pharmacol 62, 65-74 (2002). https://doi.org/10.1124/mol.62.1.65
46 Bai, H., Xie Y I Fau-Gao, Y.-X., Gao Yx Fau-Cheng, T., Cheng T Fau-Wang, Z. Z. & Wang, Z. Z. The balance of positive and negative effects of TGF-β signaling regulates the development of hematopoietic and endothelial progenitors in human pluripotent stem cells.
47 Turner, D. A. et al. Wnt/β-catenin and FGF signalling direct the specification and maintenance of a neuromesodermal axial progenitor in ensembles of mouse embryonic stem cells. Development 141, 4243-4253 (2014). https://doi.org/10.1242/dev.112979
48 Itskovitz-Eldor, J. et al. Differentiation of Human Embryonic Stem Cells into Embryoid Bodies Comprising the Three Embryonic Germ Layers. Molecular Medicine 6, 88-95 (2000). https://doi.org/10.1007/BF03401776
49 Schmidt, C. et al. Multi-chamber cardioids unravel human heart development and cardiac defects. Cell (2023). https://doi.org/https://doi.org/10.1016/j.cell.2023. 10.030
50 Ben-Yair, R. & Kalcheim, C. Lineage analysis of the avian dermomyotome sheet reveals the existence of single cells with both dermal and muscle progenitor fates.
51 Linask, K. K. N-cadherin localization in early heart development and polar expression of Na+, K(+)-ATPase, and integrin during pericardial coelom formation and epithelialization of the differentiating myocardium.
52 Borges, L. et al. A critical role for endoglin in the emergence of blood during embryonic development.
53 Sawano, A. et al. Flt-1, vascular endothelial growth factor receptor 1, is a novel cell surface marker for the lineage of monocyte-macrophages in humans.
54 Waclawiczek, A. et al. Mesenchymal niche remodeling impairs hematopoiesis via stanniocalcin 1 in acute myeloid leukemia.
55 Coe, L. M. et al. FGF-23 is a negative regulator of prenatal and postnatal erythropoiesis.
56 Imaz-Rosshandler, I. et al. Tracking early mammalian organogenesis-prediction and validation of differentiation trajectories at whole organism scale. Development (2023). https://doi.org/10.1242/dev.201867
57 Baker, S. J. et al. B-myb is an essential regulator of hematopoietic stem cell and myeloid progenitor cell development.
58 Zhou, B. O., Ding, L. & Morrison, S. J. Hematopoietic stem and progenitor cells regulate the regeneration of their niche by secreting Angiopoietin-1.
59 Soza-Ried, C., Hess I Fau-Netuschil, N., Netuschil N Fau-Schorpp, M., Schorpp M Fau-Boehm, T. & Boehm, T. Essential role of c-myb in definitive hematopoiesis is evolutionarily conserved.
60 Jowett, G. M., Coales, I. & Neves, J. F. Organoids as a tool for understanding immune-mediated intestinal regeneration and development. Development 149 (2022). https://doi.org/10.1242/dev.199904
61 Múnera, J. O. et al. Development of functional resident macrophages in human pluripotent stem cell-derived colonic organoids and human fetal colon. Cell Stem Cell 30, 1434-1451.e1439 (2023). https://doi.org/10.1016/j.stem.2023.10.002

1. An in vitro human embryo organoid at a post-gastrulation stage of development comprising:
cells expressing markers characteristic of endodermal cells,
cells expressing markers characteristic of mesodermal cells,
cells expressing markers characteristic of ectodermal cells, and
primordial germ cell-like cells (hPGCLCs).

2. The in vitro human embryo organoid at a post-gastrulation stage of development according to claim 1, wherein the in vitro human embryo organoid further comprises one or more of neuromesodermal progenitors, a heart tube, a beating heart, hindgut, neural tube, neural crest cells, neuronal progenitors, hematopoietic cells, endothelial cells, hepatocytes and cardiomyocytes.

3. The in vitro human embryo organoid at a post-gastrulation stage of development according to claim 1 or claim 2, wherein the in vitro human embryo organoid is non-integrated.

4. The in vitro human embryo organoid at a post-gastrulation stage of development according to any of claims 1 to 3, wherein in vitro human embryo organoid is cultured for any one of at least 3, 4, 5, 6, 7, 8, 9, 10, 11, 12, 13, or 14 days.

5. The in vitro human embryo organoid at a post-gastrulation stage of development according to any one of claims 1 to 4, wherein in vitro human embryo organoid is polarised along the rostro-caudal axis into an anterior region and a posterior region.

6. The in vitro human embryo organoid at a post-gastrulation stage of development according to claim 5, wherein the anterior region and posterior regions comprise differential expression of one or more one or more markers indicative of primordial germ cell-like cells.

7. The in vitro human embryo organoid at a post-gastrulation stage of development according to claim 6, wherein GATA6 gene is expressed in the anterior region and TBXT gene or CDX2 gene is expressed at the posterior region.

8. The in vitro human embryo organoid at a post-gastrulation stage of development according to claim 6 or 7, wherein the anterior region comprises upregulation or downregulation of one or more genes selected from a list comprising GATA6, MYH10, TNNI1, TNNI2, and HAND1 and/or the posterior region comprises upregulation or downregulation of one of more genes selected from a list comprising CDX2 and CDX1.

9. The in vitro human embryo organoid at a post-gastrulation stage of development according to any one of claims 6 to 8, wherein the posterior region comprises markers for neuromesodermal progenitors, wherein the markers for neuromesodermal progenitors are one or more genes selected from a list comprising TBXT, SOX2, NKX1.2, HOXB8, and HOXC9.

10. The in vitro human embryo organoid at a post-gastrulation stage of development according to any one of the preceding claims, wherein human embryo organoid comprises neural precursors, wherein the neural precursors are one or more genes selected from a list comprising SOX2, ZIC5, POU3F2, neural progenitors, wherein the neural progenitors are one or more genes selected from a list comprising ISI1, ZEB2, FOXC1, neural crest markers, wherein the neural crest are one or more genes selected from a list comprising FOXD3, S100B, HOXB9, SOX2, SOX10, CRABP1, TFAP2A, TFAP2B, neuron markers, wherein the neuron markers are one or more genes selected from a list comprising STMN2, ASCL1, ELAVL3, NEUROG1, and NEUROD4, neural tube markers, wherein the neural tube markers are one or more genes selected from a list comprising SOX3, PAX6, PAX7, and HES5, spinal cord markers, wherein the spinal cord markers are one or more genes selected from a list comprising OLIG3, SOX1, ZIC1, PAX7, and FGF8, and neural ectoderm markers, wherein the neural ectoderm markers are one or more genes selected from a list comprising SOX1 and PAX6.

11. The in vitro human embryo organoid at a post-gastrulation stage of development according to any one of the preceding claims, wherein the in vitro human embryo organoid comprises Hematopoietic Stem and Progenitor Cells (HSPC), wherein the HSPCs comprise one or more genes selected from a list comprising RUNX1, SOX7 and CD34.

12. The in vitro human embryo organoid at a post-gastrulation stage of development according to any one of the preceding claims, wherein the in vitro human embryo organoid comprises one or more selected from a list comprising hematopoietic cells CD45⁺RUNX1⁺, RUNX1⁺CD45⁻, CD45⁺RUNX1⁻, CD31⁺CD45⁻, CD31⁺CD45⁺, CD31⁻CD45⁺, CD31⁺CD34⁺CD45⁺, CD34⁺CD45⁻ and CD34⁻CD45⁺.

13. The in vitro human embryo organoid at a post-gastrulation stage of development according to claim 12, wherein the hematopoietic cells further comprise one or more genes selected from a list comprising CD45, MYB, ITGA2B, SPN, GFI1B, SPINK2 and CD44.

14. The in vitro human embryo organoid at a post-gastrulation stage of development according to any one of claim 12 or 13, wherein the hematopoietic cells further comprise markers for megakaryocytes, wherein the markers for megakaryocytes comprise one or more genes selected from a list comprising GP1BA, GP1BB and NFE2.

15. The in vitro human embryo organoid at a post-gastrulation stage of development according to any one of claims 12 to 14, wherein the hematopoietic cells further comprise markers for macrophages, wherein the markers for macrophages comprise one or more genes selected from a list comprising CD68, CD86, MRC1 and TREM2.

16. The in vitro human embryo organoid at a post-gastrulation stage of development according to any one of claims 12 to 15, wherein the hematopoietic cells further comprise markers for eosinophil, basophil and mast cell progenitors, wherein the eosinophil, basophil and mast cell progenitors comprise one or more genes selected from a list comprising GATA2, IL1B, MYC, STAT5A and SOX4.

17. The in vitro human embryo organoid at a post-gastrulation stage of development cording to any one of claims 12 to 16, wherein the hematopoietic cells further comprise one or more genes selected from a list comprising lymphocytes CD52, CD69, HOPX, SPINK2, IL7R and NKG7.

18. The in vitro human embryo organoid at a post-gastrulation stage of development according to any one of the preceding claims, wherein the in vitro human embryo organoid expresses one or more markers indicative of primordial germ cell-like cells selected from a group comprising NANOS3, POU5F1, PDPN, TFAP2C, PRDM1, PRDM14, CD38, and NANOG.

19. The in vitro human embryo organoid at a post-gastrulation stage of development according to any one of claims 1 to 12, wherein the in vitro human embryo organoid expresses one or more markers indicative of neuronal precursor cells selected from a group comprising S100B, FOXD3, SOX10, ELAVL3, NEUROD4, NEUROG1, SOX2, SOX10, CRABP1, TFAP2A, TFAP2B, STMN2, ASCL1, ELAVL3, NEUROG1, NEUROD4, SOX3, PAX6, PAX7, HES5, OLIG3, SOX1, ZIC1, PAX7, and FGF8

20. The in vitro human embryo organoid at a post-gastrulation stage of development according to claims 6 to 19, wherein the anterior region expresses TNNT2 and MYH2 genes.

21. The in vitro human embryo organoid at a post-gastrulation stage of development according to any one of the preceding claims, wherein the in vitro human embryo organoid comprises contractile cardiomyocytes, wherein the markers for cardiomyocytes are one or more genes selected from a list comprising NKX2-5 and MYH2, TNNI1, ACTC1, TNNT2, MYH6, MYL7 and IRX3.

22. The in vitro human embryo organoid at a post-gastrulation stage of development according to any one of the preceding claims, wherein the in vitro human embryo organoid comprises amnion cells, wherein the markers for amnion cells are one or more genes selected from a list comprising GABRP, VTCN1, WNT6 and TFAP2A,

23. The in vitro human embryo organoid at a post-gastrulation stage of development according to any one of the preceding claims, wherein the in vitro human embryo organoid comprises mesenchyme cells, wherein the markers for mesenchyme cells are one or more genes selected from a list comprising PCOLCE, COL1A2, COL6A3 and HAND1.

24. The in vitro human embryo organoid at a post-gastrulation stage of development according to any one of the preceding claims, wherein the in vitro human embryo organoid comprises hepatocytes, wherein the markers for hepatocytes are one or more genes selected from a list comprising AFP, ALB and CEBPA.

25. The in vitro human embryo organoid at a post-gastrulation stage of development according to any one of the preceding claims, wherein the in vitro human embryo organoid comprises blood progenitors, wherein the markers for blood progenitors are one or more genes selected from a list comprising PECAM1/CD31, CD34, CDH5, RUNX1, PTPRC/CD45 and CD44.

26. The in vitro human embryo organoid at a post-gastrulation stage of development according to any one of the preceding claims, wherein the in vitro human embryo organoid comprises splanchnic mesoderm, wherein the markers for splanchnic mesoderm are one or more genes selected from a list comprising WT1, LHX2 and GATA4.

27. The in vitro human embryo organoid at a post-gastrulation stage of development according to any one of the preceding claims, wherein the in vitro human embryo organoid comprises paraxial mesoderm, wherein the markers for paraxial mesoderm are one or more genes selected from a list comprising MEOX1, SIX4 and FOXC2.

28. The in vitro human embryo organoid at a post-gastrulation stage of development according to any one of the preceding claims, wherein the in vitro human embryo organoid comprises erythroid cells, wherein the markers for erythroid cells are one or more genes selected from a list comprising GATA1, KLF1, HBA1, HBA2, HBE1, HBE2, HBG1 and HBG2,

29. The in vitro human embryo organoid at a post-gastrulation stage of development according to any one of the preceding claims, wherein the in vitro human embryo organoid comprises endothelial cells, wherein the markers for endothelial cells are one or more genes selected from a list comprising CD34, CD31, CDH5, KDR, SOX7, THY1, ITGB1, CXCR4, NRARP, GJA4, IGFBP4, VEGF1, FLT1, TMEM88, TPM1, PRTG, HOXB7, MEIS2, RNF19A, CAV1, REN, BNIP3, STC1 and FGF23.

30. The in vitro human embryo organoid at a post-gastrulation stage of development according to any one of the preceding claims, wherein the in vitro human embryo organoid comprises hepatocytes (foetal liver progenitors), wherein the markers for hepatocytes are one or more genes selected from a list comprising AFP, APOA1, APOB, ALB and TF.

31. The in vitro human embryo organoid at a post-gastrulation stage of development according to any one of the preceding claims, wherein the in vitro human embryo organoid comprises musculoskeletal precursors, wherein the musculoskeletal precursors are one or more genes selected from a list comprising NEFM, SIX1 and TNNT1.

32. The in vitro human embryo organoid at a post-gastrulation stage of development according to any one of the preceding claims, wherein the in vitro human embryo organoid comprises hindgut, wherein the markers for hindgut are one or more genes selected from a list comprising CDX2, SOX2, SOX17, SHH and EPCAM.

33. The in vitro human embryo organoid at a post-gastrulation stage of development according to any one of the preceding claims, wherein the in vitro human embryo organoid is derived in vitro from one or more human embryonic stem cells (ESCs).

34. The in vitro human embryo organoid at a post-gastrulation stage of development according to any one of the preceding claims, wherein the in vitro human embryo organoid is derived in vitro from one or more human induced pluripotent stem cells (iPSCs).

35. A method of producing an in vitro human embryo organoid at a post-gastrulation stage of development comprising the steps of:
a) treating human embryonic stem cells or human induced pluripotent stem cells to disassociate them into single cells,
b) formation of aggregates,
c) advancement of aggregates to human embryo organoid at the peri-gastrulation stage of development; and
d) advancement of aggregates to human embryo organoid at the post-gastrulation stage of development, wherein the aggregates are incubated in the presence of a GSK3β inhibitor, preferably wherein the GSK3β inhibitor is Chiron, and a TGF-β inhibitor, preferably wherein the TGF-β inhibitor is SB43.

36. The method of producing an in vitro human embryo organoid at a post-gastrulation stage of development according to claim 35, wherein step a) is performed in the presence of a TGF-β, preferably wherein the TGF-β is Activin-A, and a GSK3β inhibitor, preferably wherein the GSK3β inhibitor is Chiron.

37. The method of producing an in vitro human embryo organoid at a post-gastrulation stage of development according to claim 35 or claim 36, wherein step b) is performed in the presence of a GSK3β inhibitor, preferably wherein the GSK3β inhibitor is Chiron.

38. The method of producing an in vitro human embryo organoid at a post-gastrulation stage of development according to any one of claims 35 to 37, wherein step c) is performed in the presence of a GSK3β inhibitor, preferably wherein the GSK3β inhibitor is Chiron, and a TGF-β inhibitor, preferably wherein the TGF-β inhibitor is SB43.

39. The method of producing an in vitro human embryo organoid at a post-gastrulation stage of development according to any one of claims 35 to 38, wherein step d) further comprises incubating the in vitro human embryo organoid in a rotary culture system.

40. The method of producing an in vitro human embryo organoid at a post-gastrulation stage of development according to any one of claims 35 to 40, wherein one or more of the method steps are performed under conditions of about 1% to about 10% oxygen and 1% to about 6% carbon dioxide, preferably about 5% oxygen and about 5% carbon dioxide, optionally, wherein all of the method steps are performed under conditions of about 1% to about 10% oxygen and 1% to about 6% carbon dioxide, preferably about 5% oxygen and about 5% carbon dioxide.

41. A method of advancing an in vitro human embryo organoid at the peri-gastrulation stage of development to a post-gastrulation stage of development comprising incubating the human embryo organoid at the peri-gastrulation stage of development in the presence of a GSK3β inhibitor, preferably wherein the GSK3β inhibitor is Chiron, and a TGF-β inhibitor, preferably wherein the TGF-β inhibitor is SB43.

42. The method of advancing an in vitro human embryo organoid at the peri-gastrulation stage of development to a post-gastrulation stage of development according to claim 41, further comprising incubating the in vitro human embryo organoid in a rotary culture system.

43. The method of advancing an in vitro human embryo organoid at the peri-gastrulation stage of development to a post-gastrulation stage of development according to any one of claims 41 to 42, wherein one or more of the method steps are performed under conditions of about 1% to about 10% oxygen and 1% to about 6% carbon dioxide, preferably about 5% oxygen and about 5% carbon dioxide, optionally, wherein all of the method steps are performed under conditions of about 1% to about 10% oxygen and 1% to about 6% carbon dioxide, preferably about 5% oxygen and about 5% carbon dioxide.

44. The method of producing an in vitro human embryo organoid at a post-gastrulation stage of development of any one of claims 35 to 40 or the method of advancing an in vitro human embryo organoid at the peri-gastrulation stage of development to a post-gastrulation stage of development according to any one of claims 22 to 24, wherein the in vitro human embryo organoid comprises:
cells expressing markers characteristic of endodermal cells,
cells expressing markers characteristic of mesodermal cells,
cells expressing markers characteristic of ectodermal cells; and
primordial germ cell-like cells (hPGCLCs).

45. The method according to claim 44, wherein the in vitro human embryo organoid further comprises one or more of neuromesodermal progenitors, a heart tube, a beating heart, hindgut, neural tube, neural crest cells, neuronal progenitors, hematopoietic cells, endothelial cells, hepatocytes, and cardiomyocytes indicative of a post-gastrulation state of development.

46. The method according to claim 44 or claim 45, wherein in vitro human embryo organoid is cultured for any one of at least 3, 4, 5, 6, 7, 8, 9, 10, 11, 12, 13, or 14 day.

47. The method according to any one of claims 44 to 46, wherein in vitro human embryo organoid is polarised along the rostro-caudal axis into an anterior region and a posterior region.

48. The method according to claim 47, wherein the anterior region and posterior regions comprise differential expression of one or more one or more markers indicative of primordial germ cell-like cells.

49. The method according to claim 48, wherein GATA6 gene is expressed in the anterior region and TBXT gene or CDX2 gene is expressed at the posterior region.

50. The method according to claim 48 or claim 49, wherein the anterior region comprises upregulation or downregulation of one of more genes selected from a list comprising GATA6, MYH10, TNNT1, TNNI2, and HAND1 and/or the posterior region comprises upregulation or downregulation of one of more genes selected from a list comprising CDX2 and CDX1.

51. The method according to any one of claims 48 to 50, wherein the posterior region comprises markers for neuromesodermal progenitors, wherein the markers for neuromesodermal progenitors are one or more genes selected from a list comprising TBXT, SOX2, NKX1.2, HOXB8, and HOXC9.

52. The method according to any one of claims 44 to 51, wherein the in vitro human embryo organoid comprises neural precursors, wherein the neural precursors are one or more genes selected from a list comprising SOX2, ZIC5, POU3F2, neural progenitors, wherein the neural progenitors are one or more genes selected from a list comprising ISI1, ZEB2, FOXC1, neural crest markers, wherein the neural crest are one or more genes selected from a list comprising FOXD3, S100B, HOXB9, SOX2, SOX10, CRABP1, TFAP2A, TFAP2B, neuron markers, wherein the neuron markers are one or more genes selected from a list comprising STMN2, ASCL1, ELAVL3, NEUROG1, and NEUROD4, neural tube markers, wherein the neural tube markers are one or more genes selected from a list comprising SOX3, PAX6, PAX7, and HES5, spinal cord markers, wherein the spinal cord markers are one or more genes selected from a list comprising OLIG3, SOX1, ZIC1, PAX7, and FGF8, and neural ectoderm markers, wherein the neural ectoderm markers are one or more genes selected from a list comprising SOX1 and PAX6.

53. The method according to any one of claims 44 to 52, wherein the in vitro human embryo organoid comprises Hematopoietic Stem and Progenitor Cells (HSPC), wherein the HSPCs comprise one or more genes selected from a list comprising RUNX1, SOX7 and CD34.

54. The method according to any one of claims 44 to 53, wherein the in vitro human embryo organoid comprises one or more selected from a list comprising hematopoietic cells CD45⁺RUNX1⁺, RUNX1⁺CD45⁻, CD45⁺RUNX1⁻, CD31⁺CD45⁻, CD31⁺CD45⁺, CD31⁻CD45⁺, CD31⁺CD34⁺CD45⁺, CD34⁺CD45⁻ and CD34⁻CD45⁺.

55. The method according to claim 54, wherein the hematopoietic cells further comprise one or more genes selected from a list comprising CD45, MYB, ITGA2B, SPN, GFI1B, SPINK2 and CD44.

56. The method according to any one of claim 54 or 55, wherein the hematopoietic cells further comprise markers for megakaryocytes, wherein the markers for megakaryocytes comprise one or more genes selected from a list comprising GP1BA, GP1BB and NFE2.

57. The method according to any one of claims 54 to 56, wherein the hematopoietic cells further comprise markers for macrophages, wherein the markers for macrophages comprise one or more genes selected from a list comprising CD68, CD86, MRC1 and TREM2

58. The method according to any one of claims 54 to 57, wherein the hematopoietic cells further comprise markers for eosinophil, basophil and mast cell progenitors, wherein the eosinophil, basophil and mast cell progenitors comprise one or more genes selected from a list comprising GATA2, IL1B, MYC, STAT5A and SOX4.

59. The method according to any one of claims 54 to 58, wherein the hematopoietic cells further comprise one or more genes selected from a list comprising lymphocytes CD52, CD69, HOPX, SPINK2, IL7R and NKG7.

60. The method according to any one of claims 44 to 59, wherein the in vitro human embryo organoid expresses one or more markers indicative of primordial germ cell-like cells selected from a group comprising NANOS3, POU5F1, PDPN, TFAP2C, PRDM1, PRDM14, CD38 and NANOG.

61. The method according to any one of claims 44 to 60, wherein the in vitro human embryo organoid expresses one or more markers indicative of neuronal precursor cells selected from a group comprising S100B, FOXD3, SOX10, ELAVL3, NEUROD4, NEUROG1, SOX2, SOX10, CRABP1, TFAP2A, TFAP2B, STMN2, ASCL1, ELAVL3, NEUROG1, NEUROD4, SOX3, PAX6, PAX7, HES5, OLIG3, SOX1, ZIC1, PAX7, and FGF8.

62. The method according to any one of claims 44 to 61, wherein the anterior region expresses TNNT2 and MYH2 genes.

63. The method according to any one of claims 44 to 62, wherein the in vitro human embryo organoid comprises contractile cardiomyocytes, wherein the markers for cardiomyocytes are one or more genes selected from a list comprising NKX2-5 and MYH2, TNNI1, ACTC1, TNNT2, MYH6, MYL7 and IRX3.

64. The method according to any one of claims 44 to 63, wherein the in vitro human embryo organoid comprises amnion cells, wherein the markers for amnion cells are one or more genes selected from a list comprising GABRP, VTCN1, WNT6 and TFAP2A,

65. The method according to any one of claims 44 to 64, wherein the in vitro human embryo organoid comprises mesenchyme cells, wherein the markers for mesenchyme cells are one or more genes selected from a list comprising PCOLCE, COL1A2, COL6A3 and HAND1.

66. The method according to any one of claims 44 to 65, wherein the in vitro human embryo organoid comprises hepatocytes, wherein the markers for hepatocytes are one or more genes selected from a list comprising AFP, ALB and CEBPA.

67. The method according to any one of claims 44 to 66, wherein the in vitro human embryo organoid comprises blood progenitors, wherein the markers for blood progenitors are one or more genes selected from a list comprising PECAM1/CD31, CD34, CDH5, RUNX1, PTPRC/CD45 and CD44.

68. The method according to any one of claims 44 to 67, wherein the in vitro human embryo organoid comprises splanchnic mesoderm, wherein the markers for splanchnic mesoderm are one or more genes selected from a list comprising WT1, LHX2 and GATA4,

69. The method according to any one of claims 44 to 68, wherein the in vitro human embryo organoid comprises paraxial mesoderm, wherein the markers for paraxial mesoderm are one or more genes selected from a list comprising MEOX1, SIX4 and FOXC2.

70. The method according to any one of claims 44 to 69, wherein the in vitro human embryo organoid comprises erythroid cells, wherein the markers for erythroid cells are one or more genes selected from a list comprising GATA1, KLF1, HBA1, HBA2, HBE1, HBE2, HBG1 and HBG2,

71. The method according to any one of claims 44 to 70, wherein the in vitro human embryo organoid comprises endothelial cells, wherein the markers for endothelial cells are one or more genes selected from a list comprising CD34, CD31, CDH5, KDR, SOX7, THY1, ITGB1, CXCR4, NRARP, GJA4, IGFBP4, VEGF1, FLT1, TMEM88, TPM1, PRTG, HOXB7, MEIS2, RNF19A, CAV1, REN, BNIP3, STC1 and FGF23.

72. The method according to any one of claims 44 to 71, wherein the in vitro human embryo organoid comprises hepatocytes (foetal liver progenitors), wherein the markers for hepatocytes are one or more genes selected from a list comprising AFP, APOA1, APOB, ALB and TF.

73. The method according to any one of claims 44 to 72, wherein the in vitro human embryo organoid comprises musculoskeletal precursors, wherein the musculoskeletal precursors are one or more genes selected from a list comprising NEFM, SIX1 and TNNT1.

74. The method according to any one of claims 44 to 73, wherein the in vitro human embryo organoid comprises hindgut, wherein the markers for hindgut are one or more genes selected from a list comprising CDX2, SOX2, SOX17, SHH and EPCAM.

75. The method according to any one of claims 44 to 74, wherein the in vitro human embryo organoid is derived in vitro from one or more human embryonic stem cells (ESCs).

76. The method according to any one of claims 44 to 74, wherein the in vitro human embryo organoid is derived in vitro from one or more human induced pluripotent stem cells (iPSCs).

77. An in vitro human embryo organoid at a post-gastrulation stage of development obtainable by a method according to any one of claims 35 to 76.

78. A progenitor cell or derivative thereof obtainable by the method according to any one of claims 35 to 76.

79. The progenitor cell or derivative thereof according to claim 78, wherein the progenitor cell or derivative thereof is an endodermal cell, mesodermal cell, ectodermal cell, neural crest cell, neuronal progenitor, neuronal precursor cell, hematopoietic cell, endothelial cell, hepatocyte cardiomyocyte, HSPC, megakaryocyte, macrophage, eosinophil, basophil, mast cell, lymphocyte, primordial germ cell-like cell, amnion cell, mesenchyme cell, hepatocytes, foetal liver progenitor, blood progenitor, erythrocyte, myeloid cell, endothelial cell, musculoskeletal precursor cell, ESC and/or iPSC.

80. A kit comprising an in vitro human embryo organoid at a post-gastrulation stage of development according to any one of claims 1 to 34.

81. A system for drug screening for teratogenic and therapeutic purposes comprising an in vitro human embryo organoid at a post-gastrulation stage of development according to any one of claims 1 to 34.
